(19) United States
(12) Patent Application Publication
(10) Pub. No.: US 2021/0319848 A1
(43) Pub. Date: Oct. 14, 2021

(54) SYSTEMS AND METHODS FOR IDENTIFYING ASSOCIATIONS BETWEEN MICROBIAL STRAINS AND PHENOTYPIC FEATURES

(71) Applicants: FRED HUTCHINSON CANCER RESEARCH CENTER, Seattle, WA (US); UNIVERSITY OF WASHINGTON, Seattle, WA (US); THE REGENTS OF THE UNIVERSITY OF MICHIGAN, Ann Arbor, MI (US)

(72) Inventors: Samuel S. MINOT, Seattle, WA (US); Amy Donaldson WILLIS, Seattle, WA (US); Jonathan Louis GOLOB, Ann Arbor, MI (US)

(21) Appl. No.: 17/225,991

(22) Filed: Apr. 8, 2021

Related U.S. Application Data
(60) Provisional application No. 63/007,278, filed on Apr. 8, 2020.

(51) Int. Cl. G16B 20/00; G16B 30/10; G16B 40/00; G16H 10/60; G16H 50/70; G01N 33/483

(52) U.S. Cl. CPC G16B 20/00; G16B 30/10; G01N 33/483; G16H 10/60; G16H 50/70; G16B 40/00

(57) ABSTRACT

Provided herein are systems and methods for identifying associations, or lack thereof, between microbial strains (e.g., bacterial strains) and phenotypic features (e.g., demographic characteristics, physical statistics, and/or medical history) of a subject.

### GOVERNMENT SUPPORT CLAUSE

This invention was made with government support under GM133420 awarded by the National Institutes of Health. The government has certain rights in the invention.

### BACKGROUND

### Technical Field

The present disclosure generally relates to identifying associations, or lack thereof, between microbial strains in the microbiome of a subject and phenotypic features of the subject.

### Description of the Related Art

Microbiome analysis provides a promising tool to better understand a range of diseases, including gastroenterological and hepatic conditions. Steps have been taken to associate particular bacteria to particular conditions have been taken. However, only limited progress has been made to advance this analysis to clinical relevance.

Accordingly, the known methods for associating bacteria with particular disease indications fall far short of being facile and clinically relevant. Improved methods for analysis of the microbiome are needed.

### BRIEF SUMMARY

As described further below, provided herein are systems and methods for identifying associations, or lack thereof, between microbial strains (e.g., bacterial strains) and phenotypic features (e.g., demographic characteristics, physical statistics, and/or medical history.

Aspects of the present disclosure include a system, comprising: (A) one or more processors; and (B) one or more computer readable hardware storage devices that comprise computer executable instructions executable by at least one of the one or more processors to cause the computer system to: (1) identify a set of physical features from a dataset comprising: (a) physical measurements of a first plurality of bacterial strains in a plurality of microbiome samples from a plurality of subjects, respectively; (b) phenotypic data comprising a phenotypic feature for each subject of the plurality of subjects; and (c) physical measurements of a second plurality of bacterial strains; (2) quantify a frequency of each physical feature of the set of physical features; (3) determine an association value for each physical feature of the set of physical features with the phenotypic feature; (4) identify a subset of bacterial strains of the second plurality of bacterial strains that is associated with the phenotypic feature, the subset of bacterial strains comprising a first bacterial strain of the second plurality of bacterial strains; and (5) output the subset of bacterial strains.

Further aspects of the present disclosure include a system, comprising: (A) one or more processors; and (B) one or more computer readable hardware storage devices that comprise computer executable instructions executable by at least one of the one or more processors to cause the computer system to: (1) identify a set of physical features from physical measurements of a first bacterial strain; (2) determine an association value for each physical feature of the set of physical features with a plurality of phenotypic features by determining a frequency of each physical feature of the set of physical features in a dataset comprising; (a) physical measurements of a plurality of microbiome samples from a plurality of subjects, respectively; (b) phenotypic data comprising the phenotypic feature for each subject of the plurality of subjects; and (c) physical measurements of a plurality of bacterial strains; and (3) identify a phenotypic feature of the plurality of phenotypic features that is associated with the first bacterial strain based at least on the association value; and (4) output the phenotypic feature.

The present disclosure further provides a system, comprising: (A) one or more processors; and (B) one or more computer readable hardware storage devices that comprise computer executable instructions executable by at least one of the one or more processors to cause the computer system to: (1) receive first physical measurements of a first plurality of bacterial strains in a microbiome sample from a subject; (2) identify a first set of physical features from the first physical measurements; (3) determine an association value for each physical feature of the first set of physical features with a plurality of phenotypic features, the association value being based on a frequency of a second set of physical features in a dataset comprising: (a) physical measurements of a second plurality of bacterial strains in a plurality of microbiome samples from a plurality of subjects, respectively; (b) phenotypic data comprising a phenotypic feature for each subject of the plurality of subjects; and (c) physical measurements of a third plurality of bacterial strains; (4) identify a phenotypic feature of the plurality of phenotypic features that is associated with the first plurality of bacterial strains based at least on the association value; and (5) output the phenotypic feature.

In further aspects, the present disclosure provides a method, comprising: (A) identifying a set of physical features from a dataset comprising: (1) physical measurements of a first plurality of bacterial strains in a plurality of microbiome samples from a plurality of subjects, respectively; (2) phenotypic data comprising a phenotypic feature for each subject of the plurality of subjects; and (3) physical measurements of a second plurality of bacterial strains; (B) quantifying a frequency of each physical feature of the set of physical features; (C) determining an association value for each physical feature of the set of physical features with the phenotypic feature; (D) identifying a subset of bacterial strains of the second plurality of bacterial strains that is associated with the phenotypic feature, the subset of bacterial strains comprising a first bacterial strain of the second plurality of bacterial strains; and (E) outputting the subset of bacterial strains.

Additional aspects of the present disclosure include a method, comprising: (A) identifying a first set of physical features from physical measurements of a first bacterial strain; (B) determining an association value for each physical feature of the first set of physical features with a plurality of phenotypic features by determining a frequency of each physical feature of the set of physical features in a dataset comprising; (1) physical measurements of a plurality of microbiome samples from a plurality of subjects, respectively; (2) phenotypic data comprising the phenotypic feature for each subject of the plurality of subjects; and (3) physical measurements of a plurality of bacterial strains; and (C) identifying a phenotypic feature of the plurality of phenotypic features that is associated with the first bacterial strain based at least on the association value; and (D) outputting the phenotypic feature.

The present disclosure also provides a method, comprising: (A) receiving first physical measurements of a first plurality of bacterial strains in a microbiome sample from a subject; (B) identifying a first set of physical features from the first physical measurements; (C) determining an association value for each physical feature of the first set of physical features with a plurality of phenotypic features, the association value being based on a frequency of a second set of physical features in a dataset comprising: (1) physical measurements of a second plurality of bacterial strains in a plurality of microbiome samples from a plurality of subjects, respectively; (2) phenotypic data comprising a phenotypic feature for each subject of the plurality of subjects; and (3) physical measurements of a third plurality of bacterial strains; (D) identifying a phenotypic feature of the plurality of phenotypic features that is associated with the first plurality of bacterial strains based at least on the association value; and (E) outputting the phenotypic feature.

Further disclosed are methods of treating a disease in a subject with a treatment regimen based on the presence of the subset of bacterial strains in a sample from the subject, the subset of bacterial strains being identified by the methods described herein. Also disclosed are methods of treatment of a disease in a subject in need thereof, the method comprising: administering a treatment regimen to the subject, wherein the subset of bacterial strains is found in a sample from the subject.

### BRIEF DESCRIPTION OF THE SEVERAL VIEWS OF THE DRAWINGS

This patent application file contains at least one drawing executed in color. Copies of this patent application publication with color drawings will be provided by the Office upon request and payment of the necessary fee.

The sizes and relative positions of elements in the figures are not necessarily drawn to scale. For example, the shapes of various elements and angles are not drawn to scale and some of these elements are arbitrarily enlarged and positioned to improve figure legibility. Further, the particular shapes of the elements as drawn, are not intended to convey any information regarding the actual shape of the particular elements, and have been solely selected for ease of recognition in the figures.

FIG. 1 illustrates a computing system according to one or more embodiments of the present disclosure.

FIG. 2 shows an example of a genome map for Crohn's Disease and E. coli SF-166 (NCBI GCF_001280385.1).

FIG. 3 shows an example of a genome map for Colorectal Cancer and Fusobacterium nucleatum subsp. nucleatum ATCC 23726.

FIGS. 4A-4C show co-abundant gene group (CAG) characteristics across a range of independent parameters selected as described in Example 3. FIG. 4A shows the distribution of CAG sizes. FIG. 4B shows the consistency of taxonomic assignment. FIG. 4C shows the distribution of cosine distances observed within each CAG in the validation dataset.

FIG. 5 shows the number of studies in which the same health measure or clinical covariate was measured as described in Example 3.

FIGS. 6A-6D show meta-analysis for gene-level association of the microbiome with serum triglyceride levels as described in Example 3. FIG. 6A provides a volcano plot showing the estimated coefficient for each microbial gene group with triglyceride levels. FIG. 6B provides an example of meta-analysis combining the results from multiple studies. FIG. 6C shows a summary of genome-level associations using the BIO-ML collection. FIG. 6D shows the triglyceride-associated genes found in each genome.

FIGS. 7A-7D show strain-level association of microbial isolates with human health as described in Example 3. Associations with triglyceride levels (FIG. 7A), BMI (FIG. 7B), colorectal cancer (FIG. 7C), and Crohn's disease (FIG. 7D) are shown.

FIG. 8 demonstrates how CAGs can be shared across species, strains, or horizontally transferred across species as described in Example 3.

FIGS. 9A and 9B show schematic diagrams of the geneshot tool for gene-level metagenomic analysis of microbiome experiments. FIG. 9A shows a description of the data flow through geneshot, with the coordinated execution of multiple constituent bioinformatic processes in order to analyze experimental data on the basis of protein-coding genes encoded by microbes present in each physical specimen. FIG. 9B shows a summary of the geneshot analysis to identify microbes associated with ICI response from published WGS datasets, indicating the number of biological features identified at each step.

FIGS. 10A-10I show genomic coordinates of ICI response-associated CAGs for Odoribacter splanchnicus DSM 220712. The relative abundance of each of four CAGs, CAG21367 (FIG. 10A), CAG31186 (FIG. 10B), CAG23495 (FIG. 10C), CAG1130 (FIG. 10I) is shown across specimens. The genomic coordinates of individual CAGs, CAG21367 (FIG. 10D), CAG31186 (FIG. 10E), and CAG23495 (FIG. 10F), are also shown. The left panel shows alignment for each gene and right panel includes text indicating the NCBI annotations in that region. The occurrence of CAGs across multiple reference genomes (FIG. 10G) and stool microbiome specimens from this dataset (FIG. 10H) are also shown. Core genomic elements are underlined in blue and accessory (or strain-specific) genetic elements, are underlined in orange.

FIGS. 11A-11E show a summary of gene-level metagenomic analysis results generated by geneshot. The results for each specimen across all cohorts is shown in comparison to the number of sequence reads for each specimen (x-position) including the number of genes detected by de novo assembly of each individual specimen (FIG. 11A), the proportion of reads from each specimen which align uniquely to the de novo gene catalog (FIG. 11B), and the number of genes from the gene catalog detected by alignment (FIG. 11C). The distribution of genes across CAGs of different sizes is shown in (FIG. 11D), with the horizontal axis showing the CAG size range and the vertical axis showing the number of genes belonging to CAGs within that size range. FIG. 11E shows the relative abundance (log 10) of the CAGs with the highest relative abundance (rows, annotated by majority taxonomic annotation) across all specimens (columns, annotated by BioProject and ICI response label), with hierarchical clustering by relative abundance across rows and columns.

FIGS. 12A-12D show visual depictions of CAG-level abundance calculation. Each CAG consists of a group of genes, each of which is detected in each specimen by alignment of WGS reads. The abundance of genes from CAG1309 (FIG. 12A) and CAG1071 (FIG. 12C) are shown in units of the absolute number of reads aligned uniquely from each specimen. The ‘depth’ of sequencing of each gene is a commonly-used length-normalized measure in which the total number of bases from all aligned reads is divided by the length of each gene. Genes are grouped into CAGs based on average linkage clustering using the cosine distance metric computed from the vector of sequencing depth values across all specimens. The proportional abundance for CAG1309 and CAG1071 are show in FIG. 12B and FIG. 12D, respectively. Proportional abundance of each CAG is computed as the sum of sequencing depth for all genes in the CAG, divided by the sum of sequencing depth of all genes detected. Specimens are arranged in alphabetical order on the horizontal axis, with abundance measures for each specimen on the vertical axis.

FIG. 13 shows the distribution of association estimates for CAGs with ICI response. Each point represents a single CAG, showing all CAGs which contain at least five constituent genes. The horizontal axis shows the estimated coefficient of association with ICI response (positive values indicate a higher relative abundance in ICI responders in comparison to progressors). The vertical axis shows the p-value for that coefficient of association for each CAG after adjustment for multiple hypothesis testing with the FDR-BH procedure.

FIGS. 14A-14E show taxonomic annotation of CAGs associated with ICI response. The taxonomic annotation of genes found in the 50 CAGs (columns) which were most consistently associated with ICI response (after applying a minimum size threshold of 30 genes), are shown in FIG. 14A with shading indicating the proportion of genes assigned to the indicated taxon (rows). The Wald statistic summarizing the association with ICI response is shown for taxa in FIG. 14B and for individual CAGs in FIG. 14D. A taxonomic tree annotating those organisms is shown in FIG. 14C, and CAG size is shown in FIG. 14G.

FIGS. 15A-15D show visual depictions of the aggregation of CAG-level ICI-response association metrics by taxon. Odoribacter splanchnicus (FIG. 15A), Coprococcus comes (FIG. 15B), Bifidobacterium longum (FIG. 15C), and Collinsella aerofaciens (FIG. 15D) are shown. For each organism, the lower panel shows the estimated coefficient and −log 10 p-value for the association of each CAG with ICI response. Every CAG either contains at least one gene which was taxonomically annotated to the indicated organism (orange), or there were no genes in that CAG with that taxonomic annotation (blue). The aggregate taxon-level ICI-response association for each organism is shown in the upper panel from each subplot, with the estimated coefficient +/−standard deviation indicated in the subplot title. The vertical grey line in each subplot marks the taxon-level estimated coefficient value.

FIGS. 16A-16G show a putative bacteriophage of Faecalibacterium prausnitzii is associated with ICI non-response, in contrast to an associated bacteriophage defense island that is associated with ICI response. The genomic coordinates of ICI response-associated CAGs are shown for two strains of F. prausnitzii, MGYG-HGUT-00195 (GCF_902364275.1; FIG. 16A) and FPSSTS7063_SV_a2_mod (GCA_902167865.1; FIG. 16B). The relative abundance CAG19205 (FIG. 16C) and CAG21565 (FIG. 16D) and a comparison of the relative abundance of CAG19205 and CAG21565 in cohort PRJNA399742 (FIG. 16E) and cohort PRJNA397906 (FIG. 16E) are shown. The ratio of relative abundances is shown as a function of ICI response across both cohorts in FIG. 16G.

FIG. 17 shows an example of CAG alignment to de novo assembly. The alignment coordinates for the alignment of a single CAG against the contigs generated by de novo assembly from this dataset is shown alongside the eggNOG functional annotations for the underlying genes. All alignments covered the entirety of the gene catalog sequence at >99% amino acid identity. The 14 kb physical region shown in this plot encompasses all such high quality alignments for this CAG in this specimen.

### DETAILED DESCRIPTION

The present disclosure provides systems and methods for identifying associations, or lack thereof, between microbial strains (e.g., bacterial strains) of a microbiome of a subject and phenotypic features (e.g., demographic characteristics (e.g., age, race, gender, etc.), physical statistics (e.g., body mass index (BMI), weight, height, etc.), and/or medical history characteristic (e.g., previous or current clinical diagnoses, allergies, etc.)) of the subject. Such systems and methods can be used to identify associations between a particular microbial strain and a subset of phenotypic features, between a particular phenotypic feature and a subset of microbial strains, and/or between a particular microbial strain and a particular phenotypic feature. Further, described herein are methods for identifying diagnostic and/or therapeutic targets based on these associations.

Prior to setting forth this disclosure in more detail, it may be helpful to an understanding thereof to provide definitions of certain terms to be used herein. Additional definitions are set forth throughout this disclosure.

As used herein, “microbe” refers to a microscopic organism contained within a sample derived from a source of interest. In embodiments, microbes are archaea bacteria, fungi, protist, micro-eukaryotes, bacteriophages, eukaryotic viruses, archaeal viruses, or other viruses. In some embodiments, a microbe described herein may be of bacterial origin. For example, a microbe may be a species of Blautia, Clostridiales, Odoribacter, Eubacterium, Faecalibacterium, Escherichia, Lactobacillus, Gemmiger, Coprococcus, or a combination thereof. In specific embodiments, the microbe comprises Odoribacter splanchnicus, Odoribacter splanchnicus DSM 220712, Blautia wexlerae, Faecalibacterium prausnitzii, Faecalibacterium sp. BIOML-A1, Eubacterium eligens, Blautia massiliensis, Escherichia coli, Clostridium sp. HMb25, Gemmiger formicilis, Coprococcus comes or a combination thereof.

“Microbiome” refers to the aggregate of all microbes found on and in a subject's body.

As used herein “microbiome survey” refers to a collection of samples from subjects (e.g., human subjects) that includes data regarding phenotypic feature(s) of the subjects.

As used herein, “metagenomics” refers to the using modern genomic analysis techniques to profile the composition of microbial species within a sample using a genome sequencing instrument.

As used herein, the term “sample” refers to a suitable biological specimen obtained or derived from a subject. As is understood, suitable biological specimens may comprise blood, stool, urine, tissue, cells, cell cultures, saliva, and the like. For example, a sample may comprise a stool sample from a human subject with a phenotypic feature of interest, such as colon cancer or Crohn's Disease.

“Subject” includes humans, domestic animals, such as laboratory animals (e.g., dogs, monkeys, rats, mice, etc.), household pets (e.g., cats, dogs, rabbits, etc.), and livestock (e.g., pigs, cattle, sheep, goats, horses, etc.), and non-domestic animals (e.g., bears, elephants, porcupines, etc.). In embodiments, a subject is a mammal. In embodiments, a subject is a human.

As used herein, the term “phenotypic feature” refers to a characteristic of the subject from which the sample was obtained. Any suitable health feature may be a phenotypic feature, such as a demographic characteristic (e.g., age, race, gender, etc.), physical statistic (e.g., measurements of body mass index (BMI), blood pressure, high-density lipoproteins (HDL), low-density lipoproteins (LDL), triglycerides, weight, height, calprotectin, cholesterol, high-sensitivity C-reactive protein (hs-CRP), etc.), and/or medical history characteristic (e.g., previous or current diseases or conditions (e.g., clinical diagnoses, allergies, etc.)). In embodiments, the diseases or conditions include gastroenterological and/or hepatological conditions. In embodiments, the diseases or conditions include cancer. In embodiments, the clinical diagnoses include diagnoses of gastroenterological (e.g., cancers of the esophagus, pancreas, stomach, colon, rectum, anus, liver, biliary system, and small intestine; irritable bowel syndrome (IBS); Crohn's disease; etc.) and/or hepatological conditions (e.g., cancers of the liver or pancreas; non-alcoholic fatty liver disease (NAFLD); nonalcoholic steatohepatitis (NASH); alcoholic steatohepatitis; etc.). In some embodiments, the clinical diagnoses include adenomas. In some embodiments, the phenotypic feature is a disease or condition that is responsive to a particular treatment regimen. For example, in some embodiments, a phenotypic feature is a cancer that is responsive to immune checkpoint inhibitor (ICI)-based therapy. In further embodiments, a phenotypic feature is a cancer that is responsive to radiation treatment. In further embodiments, a phenotypic feature is a cancer that is responsive to surgical intervention. Examples of specific phenotypic features include diagnosis of colorectal cancer, diagnosis of Crohn's Disease, age, and BMI.

As is understood BMI refers to a human's weight in kilograms divided by the square of height in meters. A BMI value below 18.5 refers to a subject that is underweight; a BMI value from 18.5 to 24.9 refers to a subject that is normal weight; a BMI value of 25-29.9 refers to a subject that is overweight; and a BMI value of at least 30.0 refers to a subject that is obese. When referred to herein, “BMI” could reference either a specific value or a classification of underweight, normal weight, etc. based on the specific value.

As used herein, the term “microbial strain” refers to a subset of a microbial species that differ from other subsets of the same species by a minor but identifiable difference. Similarly, the term “bacterial strain” refers to a subset of a bacterial species that differ from other subsets of the same species by a minor but identifiable difference. For example, within the present invention, a bacterial strain of the species E. coli isolated from a sample may contain a plasmid that is not present in all E. coli and is not required for a microbe to be classified as E. coli. Additionally, as used herein, “bacterial strains” include “bacterial isolates,” the latter of which refers to strains of a bacteria that has been cultured in isolation in laboratory conditions.

As used herein, “physical features” of a microbe refer to a characteristic that can be measured, such as a genomic elements (such as genes, DNA sequence motifs, k-mers (i.e., subsequences of length ‘k’ contained within a biological sequence), hidden Markov model (HMM) sequence signatures) or non-genomic elements (e.g., as measured by microscopy or mass spectrometry).

As used herein, the term “gene” refers to a nucleic acid sequence derived from a microbe that contains information necessary for expression of a polypeptide, protein, or untranslated RNA and that can be used as an analytical unit in the comparison of microbial genomes.

As used herein, the term “genomic island” refers to a contiguous region of nucleic acid sequence derived from a microbe. In some embodiments, a genomic island is about 10 kilobases to about 35 kilobases in size. In some embodiments, a genomic island contains genes. In some embodiments, a genomic island contains Co-Abundant Gene groups.

As used herein, the term “operon” refers to a unit made up of linked genes. For example, an operon may contain a cluster of genes under the control of one promoter. An operon may further contain multiple structural genes or cistrons. An operon provides means for coordinated expression of functionally related proteins.

As used herein, “frequency” refers to a quantitative measure of the number of times a microbial feature or gene appears in a sample. In some embodiments, frequency is determined by Whole-Genome Shotgun (WGS) sequencing and an alignment-based approach.

As used herein, “nucleic acid” or “nucleic acid molecule” refers to deoxyribonucleic acid (DNA), ribonucleic acid (RNA), or a combination thereof. For example, nucleic acid molecules (e.g., oligonucleotides), including those generated by the polymerase chain reaction (PCR) or by in vitro transcription, and to those generated by any of ligation, scission, endonuclease action, or exonuclease action. In certain embodiments, the nucleic acids of the present disclosure are produced by PCR. Nucleic acids can be composed of monomers that are naturally occurring nucleotides (such as deoxyribonucleotides and ribonucleotides), analogs of naturally occurring nucleotides (e.g., a-enantiomeric forms of naturally occurring nucleotides), or a combination of both. Modified nucleotides can have modifications in or replacement of sugar moieties, or pyrimidine or purine base moieties. In embodiments, modified nucleic acids are peptide nucleic acids (PNA). Modified nucleic acids can include modified backbone residues or linkages that are synthetic, naturally occurring, or non-naturally occurring, and which have similar binding properties as a reference naturally occurring nucleic acid, and which are metabolized in a manner similar to the reference nucleic acid. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, methyl phosphonate (e.g., chiral methyl phosphonate), 2-O-methyl ribonucleotide, and the like. In various embodiments, modified internucleotide linkages are used. Modified internucleotide linkages are well known in the art and include methylphosphonates, phosphorothioates, phosphorodithionates, phosphoroamidites and phosphate ester linkages. Nucleic acid molecules can be either single stranded or double stranded. Additionally, nucleic acid molecules can refer to sense or anti-sense strands, cDNA, genomic DNA, recombinant DNA, RNA, mRNA, naturally occurring molecules, and wholly or partially synthesized nucleic acid molecules. In specific embodiments, the nucleic acid molecules are genomic DNA.

The terms “nucleotide sequence” or “nucleic acid sequence” refer to the order of nucleotides in a heteropolymer of nucleotides.

“Sequence identity,” as used herein, refers to the percentage of nucleic acid or amino acid residues in one sequence that are identical with the residues in a reference sequence after aligning the sequences and introducing gaps, if necessary, to achieve the maximum percent sequence identity. The percentage sequence identity values can be generated using the DIAMOND software (Buchfink, et al., Nature Methods, 2015, 12, 59-60), with the parameters set to default values. “Substantially identical” refers to a peptide or nucleic acid molecule exhibiting at least 50% identity to a reference amino acid sequence or nucleic acid sequence, respectively. In embodiments, such a sequence is at least 60%, 80%, 85%, 90%, 95%, or 99% identical at the amino acid or nucleic acid level to the reference sequence.

Nucleic acid molecules having “substantial identity” to a sequence are typically capable of hybridizing with the complement of the sequence.

A “cancer,” including a “tumor,” refers to an uncontrolled growth of cells and/or abnormal increased cell survival and/or inhibition of apoptosis which interferes with the normal functioning of the bodily organs and systems. “Cancer” (e.g., a tumor) includes solid and non-solid cancers. A subject that has a cancer or a tumor has an objectively measurable number of cancer cells present in the subject's body. “Cancers” include benign and malignant cancers (e.g., benign and malignant tumors, respectively), as well as dormant tumors or micrometastases. “Cancers” include hematologic cancers and solid tumors. Exemplary cancers include acute lymphoblastic leukemia (ALL), acute myeloid leukemia (AML), adrenocortical carcinoma, AIDS-related cancers, anal cancer, appendix cancer, astrocytoma (e.g. childhood cerebellar or cerebral), basal-cell carcinoma, bile duct cancer, bladder cancer, bone tumor (e.g. osteosarcoma, malignant fibrous histiocytoma), brainstem glioma, brain cancer, brain tumors (e.g. cerebellar astrocytoma, cerebral astrocytoma/malignant glioma, ependymoma, medulloblastoma, supratentorial primitive neuroectodermal tumors, visual pathway and hypothalamic glioma), breast cancer, bronchial adenomas/carcinoids, Burkitt's lymphoma, carcinoid tumors, central nervous system lymphomas, cerebellar astrocytoma, cervical cancer, chronic lymphocytic leukemia (CLL), chronic myelogenous leukemia (CIVIL), chronic myeloproliferative disorders, colon cancer, cutaneous t-cell lymphoma, desmoplastic small round cell tumor, endometrial cancer, ependymoma, esophageal cancer, Ewing's sarcoma, extracranial germ cell tumor, extragonadal germ cell tumor, extrahepatic bile duct cancer, eye cancer, gallbladder cancer, gastric (stomach) cancer, gastrointestinal stromal tumor (GIST), germ cell tumor (e.g. extracranial, extragonadal, ovarian), gestational trophoblastic tumor, gliomas (e.g. brain stem, cerebral astrocytoma, visual pathway and hypothalamic), gastric carcinoid, head and neck cancer, heart cancer, hepatocellular (liver) cancer, hypopharyngeal cancer, hypothalamic and visual pathway glioma, intraocular melanoma, islet cell carcinoma (endocrine pancreas), kidney cancer (renal cell cancer), laryngeal cancer, leukemias (e.g. acute lymphocytic leukemia, acute myelogenous leukemia, chronic lymphocytic leukemia, chronic myeloid leukemia, hairy cell), lip and oral cavity cancer, liposarcoma, liver cancer, lung cancer (e.g. non-small cell, small cell), lymphoma (e.g. AIDS-related, Burkitt, cutaneous T-cell Hodgkin, non-Hodgkin, primary central nervous system), medulloblastoma, melanoma, Merkel cell carcinoma, mesothelioma, metastatic squamous neck cancer, mouth cancer, multiple endocrine neoplasia syndrome, multiple myeloma, mycosis fungoides, myelodysplastic syndromes, myelodysplastic/myeloproliferative diseases, myelogenous leukemia, myeloid leukemia, myeloid leukemia, myeloproliferative disorders, chronic, nasal cavity and paranasal sinus cancer, nasopharyngeal carcinoma, neuroblastoma, non-Hodgkin lymphoma, non-small cell lung cancer, oral cancer, oropharyngeal cancer, osteosarcoma, ovarian cancer, pancreatic cancer, pancreatic cancer, paranasal sinus and nasal cavity cancer, parathyroid cancer, penile cancer, pharyngeal cancer, pheochromocytoma, pineal astrocytoma and/or germinoma, pineoblastoma and supratentorial primitive neuroectodermal tumors, pituitary adenoma, plasma cell neoplasia/multiple myeloma, pleuropulmonary blastoma, primary central nervous system lymphoma, prostate cancer, rectal cancer, renal cell carcinoma (kidney cancer), renal pelvis and ureter, retinoblastoma, rhabdomyosarcoma, salivary gland cancer, sarcoma (e.g. Ewing family, Kaposi, soft tissue, uterine), Sézary syndrome, skin cancer (e.g. nonmelanoma, melanoma, merkel cell), small cell lung cancer, small intestine cancer, soft tissue sarcoma, squamous cell carcinoma, squamous neck cancer, stomach cancer, supratentorial primitive neuroectodermal tumor, t-cell lymphoma, testicular cancer, throat cancer, thymoma and thymic carcinoma, thyroid cancer, trophoblastic tumors, ureter and renal pelvis cancers, urethral cancer, uterine cancer, uterine sarcoma, vaginal cancer, visual pathway and hypothalamic glioma, vulvar cancer, Waldenström macroglobulinemia, or Wilms tumor.

“Metastasis” refers to the spread of cancer from its primary site to other places in the body. “Metastases” are cancers which migrate from their original location and seed vital organs, which can eventually lead to the death of the subject through the functional deterioration of the affected organs. Metastasis is a sequential process, where cancer cells can break away from a primary tumor, penetrate into lymphatic and blood vessels, circulate through the bloodstream, and grow in a distant focus (metastasize) in normal tissues elsewhere in the body. At the new site, the cells establish a blood supply and can grow to form a life-threatening mass. Metastasis can be local or distant. Both stimulatory and inhibitory molecular pathways within the tumor cell regulate this behavior, and interactions between the tumor cell and host cells in the new site are also significant.

“Treating” or “treatment” as used herein refers to the administration of a medication or medical care to a subject, such as a human, having a disease or condition of interest, e.g., a cancer, including: (i) preventing the disease or condition from occurring in a subject, in particular, when such subject is predisposed to the condition but has not yet been diagnosed as having it; (ii) inhibiting the disease or condition, i.e., arresting its development; (iii) relieving the disease or condition, i.e., causing regression of the disease or condition; or (iv) relieving the symptoms resulting from the disease or condition, (e.g., pain, weight loss, cough, fatigue, weakness, etc.) without addressing the underlying disease or condition. As used herein, the terms “disease” and “condition” may be used interchangeably or may be different in that the particular malady or condition may not have a known causative agent (so that etiology has not yet been confirmed) and it is therefore not yet recognized as a disease but only as an undesirable condition or syndrome, wherein a more or less specific set of symptoms have been identified by clinicians.

“Effective amount” refers to the amount of a compound or composition which, when administered to a subject, such as a human, is sufficient to effect treatment of the subject's cancer. The amount of a compound or composition that constitutes an “effective amount” will vary depending on the compound or composition, the condition being treated and its severity, the manner of administration, the duration of treatment, and/or the age of the subject to be treated, but can be determined routinely by one of ordinary skill in the art based on his own knowledge and this disclosure. In embodiments, an “effective amount” effects treatment (e.g., treats, prevents, inhibits, relieves, promotes, improves, increases, reduces, and the like) as measured by a statistically significant change in one or more indications, symptoms, signs, diagnostic tests, vital signs, and the like. In other embodiments, an “effective amount” suppresses, manages, or prevents a condition as measured by a lack of a statistically significant change in one or more indications, symptoms, signs, diagnostic tests, vital signs, and the like.

“Reference” refers to a gene, sequence, sample, dataset, etc. that is used as a basis for comparison.

As used herein, “statistically significant” refers the probability of obtaining results at least as extreme as the observed results assuming the null hypothesis that there is no true association, which can be measured using a p value with an appropriate statistical test. Lower p values indicate that it is unlikely that a particular event or result being measured has arisen by chance. Statistical significance may be calculated using, for example, a corncob algorithm (Martin, et al., arXiv:1902.02776 [stat.ME]).

Unless clearly indicated otherwise, as used herein, the term “or” is understood to be inclusive. Unless specifically stated or obvious from context, as used herein, the terms “a”, “an”, and “the” are understood to be singular or plural.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments. In other words, the term “consisting essentially of” limits the scope of a claim to the specified materials or steps, or to those that do not materially affect the basic characteristics of a claimed invention. Any systems or methods provided herein can be combined with one or more of any of the other systems and methods provided herein.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size, or thickness, are to be understood to include any integer within the recited range, unless otherwise indicated.

Unless otherwise clear from context, all numerical values provided herein are modified by the term about. Unless clearly indicated otherwise, as used herein, the term “about” is understood as within a range of normal tolerance in the art, for example within 2 standard deviations of the mean. As used herein, the term “about” means±20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives. As used herein, the terms “include,” “have” and “comprise” are used synonymously.

“Optional” or “optionally” means that the subsequently described event or circumstances may or may not occur, and that the description includes instances where said event or circumstance occurs and instances in which it does not.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991).

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the nucleic acid molecules and peptides of the invention, and, as such, may be considered in making and practicing the invention. Particularly useful techniques for particular embodiments will be discussed in the sections that follow.

In the following description, certain specific details are set forth in order to provide a thorough understanding of various disclosed implementations. However, one skilled in the relevant art will recognize that implementations may be practiced without one or more of these specific details, or with other methods, components, materials, etc. In other instances, well-known structures associated with computer systems, server computers, and/or communications networks have not been shown or described in detail to avoid unnecessarily obscuring descriptions of the implementations.

Described herein are systems and methods to analyze data from microbiome studies and reference microbial strains (e.g., bacterial strains) in order to associate phenotypic features and particular microbial strains. Such systems and methods can be used to microbial strains (e.g., bacterial strains) that are associated with phenotypic features (e.g., demographic characteristics (e.g., age, race, gender, etc.), physical statistics (e.g., body mass index (BMI), weight, height, etc.), and/or medical history characteristic (e.g., previous or current clinical diagnoses, allergies, etc.)), as well as to identify genes or biological functions within the microbial strain that are associated with the particular phenotypic feature.

Additionally, such associations may be positive or negative. In other words, the presence of a phenotypic feature may have a statistically significant correlation with the higher abundance of a particular microbial strain (i.e., a positive association). Alternatively, the absence of a phenotypic feature may have a statistically significant correlation with the higher abundance of a particular microbial strain (i.e., a negative association). Additionally, the systems and methods described herein may be used to support the lack of association between a phenotypic feature and a particular microbial strain.

Advantageously, the methods of the present disclosure allow for distinction between various microbial strains in such associations. As is understood, individual microbes have slight differences at the genomic level. For example, different strains of bacteria from the same species share a large amount of genomic content, but may also contain genes that are unique to a particular strain and that may encode for some phenotypically meaningful functions. In a specific example, bacterial strains classified as being in the species E. coli contain a large “core” genome which is found across all E. coli strains. However, there is an equally large number of “auxiliary” genes which are found sporadically across strains. Some of the auxiliary genes encode for toxins or secretion systems that can directly impact the health of a human host (e.g., shiga toxin, which causes the disease shigella, and which is encoded by a horizontally transferred genomic element).

The systems and methods described herein utilize a dataset that comprises physical measurements for a bacterial strains in a microbiome sample from a subject. The physical measurements may be any suitable measurement that can be associated with a particular phenotypic feature and assigned a p value. For example, the physical measurement is of a genomic element (e.g., genomic sequence data, DNA sequence motifs, k-mers, HMM sequence signatures, etc.). In other embodiments, the physical measurement is of a non-genomic element, and is generated by microscopy or mass spectrometry.

In particular embodiments, the physical measurements comprise genomic sequence data produced, for example, by Whole-Genome Shotgun (WGS). As used herein, WGS sequencing refers to a method for sequencing whole genomes at one time through the random fragmentation, cloning and sequencing (R. Staden et. al. 1979 and E. D. Green et. al. 2001). In various embodiments, the physical measurements are for a plurality of bacterial strains from a plurality of microbiome samples from a plurality of subject, respectively.

Additionally, the dataset comprises phenotypic data comprising a phenotypic feature for each subject of the plurality of subjects. The phenotypic data include any suitable health features, as discussed above, and may include demographic characteristics (e.g., age, race, gender, etc.), physical statistics (e.g., body mass index (BMI), weight, height, etc.), and/or medical history characteristic (e.g., previous or current diseases or conditions (e.g., clinical diagnoses, allergies, etc.)). In embodiments, the diseases or conditions include gastroenterological and/or hepatological conditions. In embodiments, the diseases or conditions include cancer. In embodiments, the clinical diagnoses include diagnoses of gastroenterological (e.g., cancers of the esophagus, pancreas, stomach, colon, rectum, anus, liver, biliary system, and small intestine; irritable bowel syndrome (IBS); Crohn's disease; etc.) and/or hepatological conditions (e.g., cancers of the liver or pancreas; non-alcoholic fatty liver disease (NAFLD); nonalcoholic steatohepatitis (NASH); alcoholic steatohepatitis; etc.). In some embodiments, the phenotypic feature is a disease or condition that is responsive to a particular treatment regimen. For example, in some embodiments, a phenotypic feature is a cancer that is responsive to immune checkpoint inhibitor ICI-based therapy. In further embodiments, a phenotypic feature is a cancer that is responsive to radiation treatment. In further embodiments, a phenotypic feature is a cancer that is responsive to surgical intervention. In particular embodiments, the phenotypic feature is a cancer (e.g., melanoma). In specific embodiments, the phenotypic feature is a cancer that is responsive to a particular treatment regimen (e.g., melanoma responsive to ICI-based therapy.

Further, the dataset comprises physical measurements for a second plurality of bacterial strains. In embodiments, the physical measurements comprise genomic sequence data (e.g., metagenomic data) produced. The second plurality of bacterial strains may be characterized using the same methods as the bacterial strains in microbiome sample(s) referenced above. In particular embodiments, the physical measurements are obtained using WGS. Alternatively, or in addition, the physical measurements may be obtained from existing databases (e.g., RefSeq (NCBI, Bethesda Md.), BIO-ML (the Broad Institute, Cambridge, Mass.), etc.).

In embodiments, a set of physical features is identified in the dataset. In various embodiments, the set of physical features comprises a collection of bacterial genes that may be present in a sample from a subject.

In some embodiments, the set of physical features is produced by extracting gene information from reference genomes, such as those that are available in existing databases. In specific embodiments, the set of physical features is identified by (1) assembling metagenomic data e.g. using MEGAHIT (Li, et al., Bioinformatics. 2015 May 15; 31(10):1674-6); (2) identifying protein-coding gene sequences, e.g. using Prodigal (Hyatt, et al., BMC Bioinformatics. 2010; 11: 119); (3) dereplicating gene sequences across samples e.g. using MMseqs2 (Steinegger, et al., Nat Biotechnol. 2017, 35, 1026-1028); and (4) outputting the data. In further embodiments, the set of physical features is produced by performing steps (1) and (2) above on long read sequencing information, produced, for example using PacBio (Rhoads, et al, Genomics, Proteomics & Bioinformatics, 2015, 13:5, 278-289), Oxford Nanopore Technologies (ONT; Oxford, UK), Illumina sequencing, and the like.

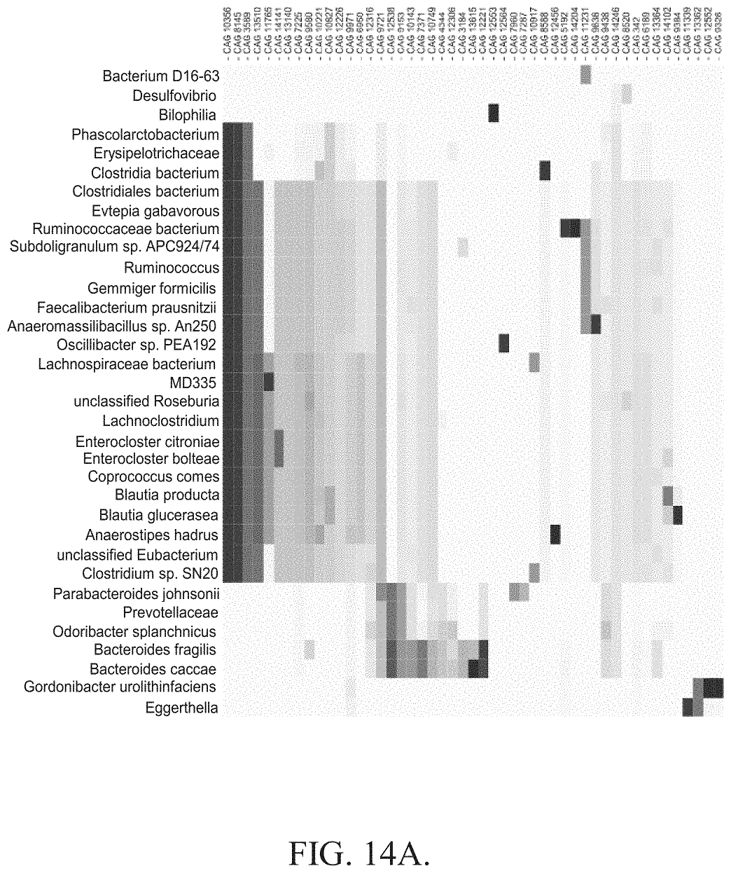

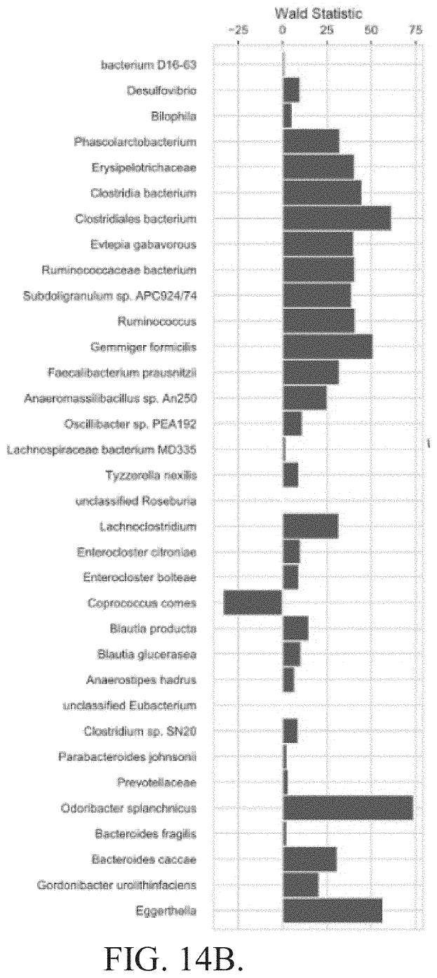

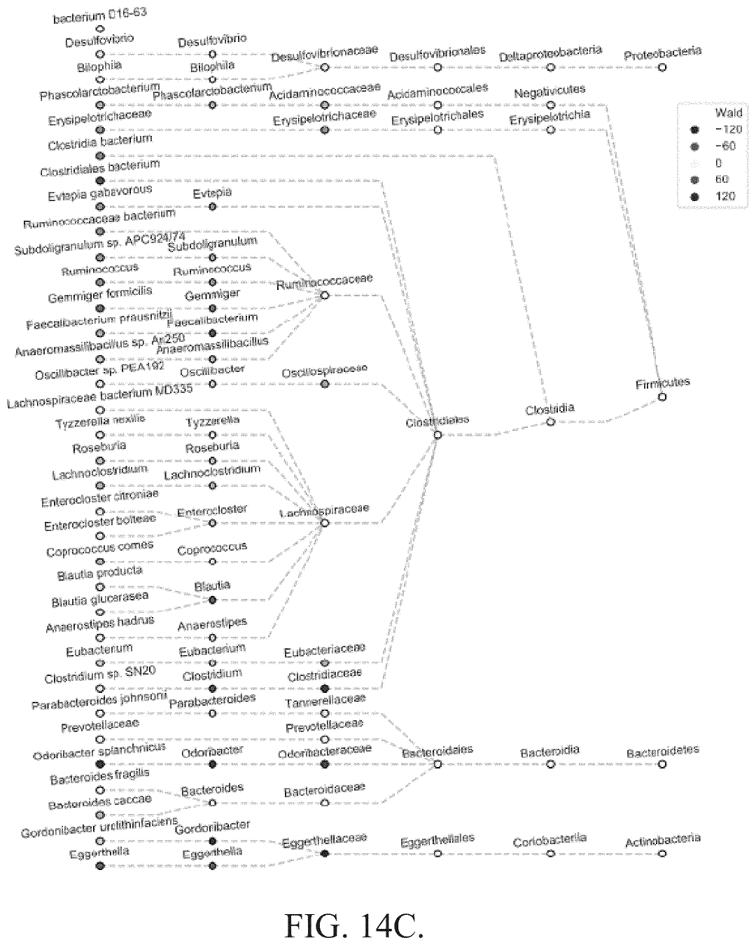

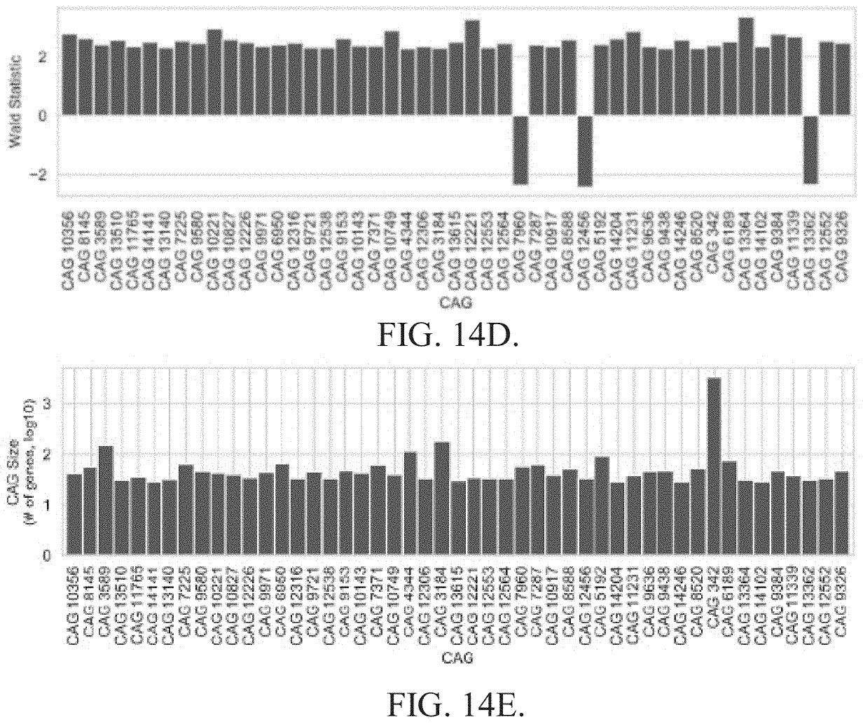

In embodiments, the set of physical features comprising a collection of bacterial genes is aligned to a set of bacterial reference genomes (see FIG. 14C) to identify genomic islands. In particular embodiments, the alignment approach comprises: Aligning genes from the geneshot output against a set of reference genomes using the Annotation of Microbial Genomes by Microbiome Association (AMGMA) tool, which aligns genes from the geneshot gene catalog against a user-specified set of microbial genomes and then calculates summary metrics for each genome including the proportion of the genome which is aligned by genes which are associated with a given parameter. Alignment is performed using DIAMOND (using the dependencies shown in Table 3; Buchfink, et al., Nature Methods, 2015, 12, 59-60) with the complete set of Python code used to aggregate alignment results available (github.com/FredHutch/AMGMA/). In embodiments, the genomic islands are about 10 kb to about 35 kb in size. In embodiments, the genomic islands are contained within particular operons.

In embodiments, a frequency of each physical feature is quantified. Any suitable approach may be used to quantify the frequency. For example, in embodiments, where the physical features are genes, an alignment-based approach is used. In particular embodiments, the alignment based approach comprises: (1) aligning sequencing reads (e.g., WGS reads) against the reference genes using, e.g., DIAMOND (Buchfink, et al., Nature Methods, 2015, 12, 59-60); (2) filtering alignments to eliminate multi-mapping reads using an algorithm using, e.g., FAMLI (Golob, et al., bioRxiv 295352, 2018, https://doi.org/10.1101/295352); (3) calculating the frequency of each gene across the sample set; and (4) outputting a data matrix with a frequency metric for each gene, based on the number of reads or the average (length-adjusted) depth of sequencing across each gene).

In further embodiments, the frequency is quantified by using the frequency information generated by de novo assembly of a data set as described above to yield the abundance of each feature in the sample. In still further embodiments, the frequency is quantified by computing the number of long reads (e.g., using ONT or PacBio) which have significant sequence identity to each reference gene. In yet further embodiments, quantification of the frequency of other physical features (e.g., those measured via microscopy or mass spectrometry) can be performed using standard statistical methods.

In embodiments, an association value is determined for each physical feature with the phenotypic feature of the subject. The association value may be determined using any suitable statistical methods (i.e., a statistical method that controls the Type I error rate of hypothesis tests). In some embodiments, dimensionality reduction may be performed to mitigate the burden of multiple hypothesis testing.

In particular embodiments, the association is determined using a method comprising: (1) grouping genes by average linkage clustering (Minot, et al., 2019 doi: 10.1186/s40168-019-0722-6) to form “Co-Abundant Gene Groups” (CAGs), using a cosine distance threshold of 0.5 (the metric ranges from 0-1); (2) running a corncob algorithm (Martin, et al., arXiv:1902.02776 [stat.ME]), which tests for differences in proportional abundance of individual gene groups, while taking into account the challenges of sparse observations as well as the relationship between measurement uncertainty and the absolute number of reads measured per gene, on each CAG; and (3) output a table with the estimated coefficient of association, standard deviation of the estimated coefficient, and p-value for the significance of the observed association, for every element in the statistical model, and for every CAG being tested, as well as a table describing which genes are found in which CAGs. An example of such a table is provided as Table 1.

TABLE 1

| Microbiome | | Estimated | Standard | |
|---|---|---|---|---|
| Feature | Phenotype | Coefficient | Deviation | p-value |
| CAG-1 | Age | 0.1 | 0.15 | 0.2 |
| CAG-1 | BMI | −0.3 | 0.001 | 0.00056 |
| CAG-2 | Age | 0.98 | 1.5 | 0.5 |
| CAG-2 | BMI | 0.001 | 0.00073 | 0.0098 |

In embodiments, an association between a particular microbial strain and a phenotypic feature is identified. In some embodiments, an association between a subset of bacterial strains and a particular phenotypic feature is identified. In some embodiments, an association between a particular bacterial strain and a subset of phenotypic features is identified. In some embodiments, an association between a particular bacterial strain and a particular phenotypic feature is identified.

In embodiments that use the method of determining an association value described above, the CAG-level association value are linked to individual microbial genomes using the intermediate element of the microbial gene. Every CAG is made up of genes, and each gene in the CAG can be detected in every microbial genome. Each gene is included in a single CAG. In some embodiments, a determination as to whether two sequences are the same gene is made. In embodiments, this is determined by sequence similarity. In specific embodiments, two sequences are considered to be the same gene if they share at least 80% sequence identity. In particular embodiments, two sequences are considered to be the same gene if they share at least 90%, 91%, 92%, 93%, 94%, 95%, 96%, 97%, 98%, 99%, or 100% sequence identity. In other embodiments, two sequences considered to the same gene based at least in part on secondary or tertiary structure.

In particular embodiments, identifying an association between a bacterial strain and a phenotypic feature comprises: (1) identifying genes in microbial genomes by alignment of either the nucleotide sequence, or the predicted coding protein sequences (e.g., using DIAMOND), and then applying a sequence identity and coverage threshold to that set of alignments; (2) for each individual genome, annotating the genome with the association metrics for each gene according to the CAG to which the gene is included; (3) calculating summary metrics for each genome (e.g., the average p value for each gene in the genome; the average estimated coefficient for each gene in the genome; or any other aggregate metric based on p values and estimated coefficients for each gene in the genome); and (4) outputting a set of summary metrics for each individual microbe, as well as a genome “map” in which the physical location of genes is combined with the association metrics for those genes against the phenotype of interest.

In various embodiments, different summary metrics, methods of determining if two sequences are the same gene, different algorithms or methods for identifying genes from microbial genomes, or a combination thereof, may be used.

In embodiments, the methods and systems described herein are advantageously used in methods of identifying microbial strains that can be used as probiotics or therapeutic agents. In further embodiments, the methods and systems described herein are advantageously used in methods of confirming that a potential probiotic or therapeutic agent is not associated with an undesirable phenotypic feature (e.g., colorectal cancer). In still further embodiments, the methods and systems described herein are advantageously used in methods of identifying candidate functional pathways or molecular elements which form the mechanism of host-microbiome interactions. In yet further embodiments, the methods and systems described herein are advantageously used in methods of identifying identify targets for treating pathogenic bacteria, e.g., using antibodies or bacteriophage. In even further embodiments, the methods and systems described herein are advantageously used in methods of genetically engineering the microbiome, e.g., by introducing genetically modified bacterial to a subject's microbiome.

It should be appreciated that one or more method acts or steps disclosed herein, can be implemented on one or more computing systems.

Computing systems are increasingly taking a wide variety of forms. Computing systems may, for example, be handheld devices, appliances, laptop computers, desktop computers, mainframes, distributed computing systems, datacenters, or even devices that have not conventionally been considered a computing system, such as wearables (e.g., glasses, watches). In this description and in the claims, the term “computing system” is defined broadly as including any device or system—or combination thereof—that includes at least one physical and tangible processor and a physical and tangible memory capable of having thereon computer-executable instructions that may be executed by a processor. The memory may take any form and may depend on the nature and form of the computing system. A computing system may be distributed over a network environment and may include multiple constituent computing systems.

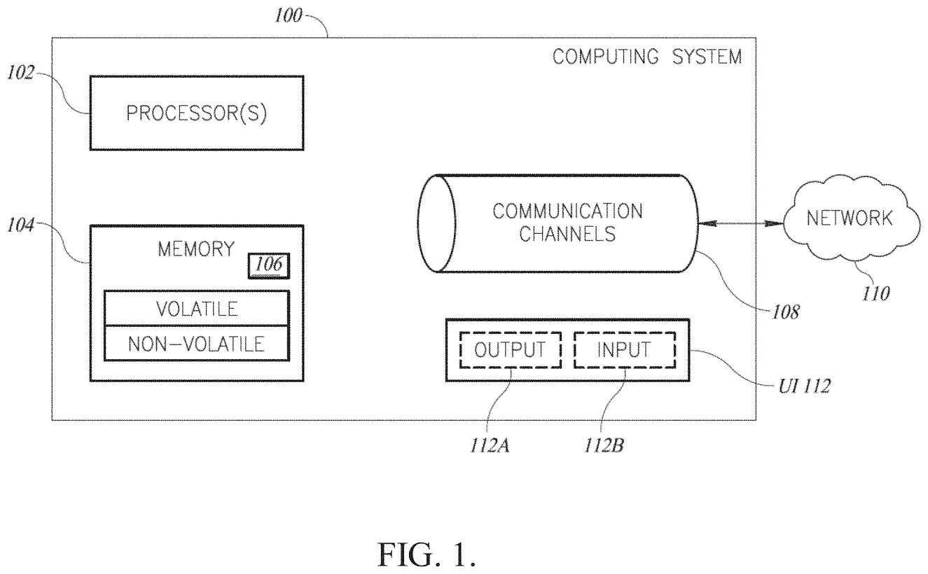

As illustrated in FIG. 1, in its most basic configuration, a computing system 100 typically includes at least one hardware processing unit 102 and memory 104. The memory 104 may be physical system memory, which may be volatile, non-volatile, or some combination of the two. The term “memory” may also be used herein to refer to non-volatile mass storage such as physical storage media. If the computing system is distributed, the processing, memory, and/or storage capability may be distributed as well.

The computing system 100 also has thereon multiple structures often referred to as an “executable component.” For instance, the memory 104 of the computing system 100 is illustrated as including executable component 106. The term “executable component” is the name for a structure that is well understood to one of ordinary skill in the art in the field of computing as being a structure that can be software, hardware, or a combination thereof. For instance, when implemented in software, one of ordinary skill in the art would understand that the structure of an executable component may include software objects, routines, methods, and so forth, that may be executed on the computing system, whether such an executable component exists in the heap of a computing system, or whether the executable component exists on computer-readable storage media.

In such a case, one of ordinary skill in the art will recognize that the structure of the executable component exists on a computer-readable medium such that, when interpreted by one or more processors of a computing system (e.g., by a processor thread), the computing system is caused to perform a function. Such structure may be computer-readable directly by the processors—as is the case if the executable component were binary. Alternatively, the structure may be structured to be interpretable and/or compiled—whether in a single stage or in multiple stages—so as to generate such binary that is directly interpretable by the processors. Such an understanding of exemplary structures of an executable component is well within the understanding of one of ordinary skill in the art of computing when using the term “executable component.”

The term “executable component” is also well understood by one of ordinary skill as including structures that are implemented exclusively or near-exclusively in hardware, such as within a field programmable gate array (FPGA), an application specific integrated circuit (ASIC), Program-specific Standard Products (ASSPs), System-on-a-chip systems (SOCs), Complex Programmable Logic Devices (CPLDs), or any other specialized circuit. Accordingly, the term “executable component” is a term for a structure that is well understood by those of ordinary skill in the art of computing, whether implemented in software, hardware, or a combination. In this description, the terms “component,” “service,” “engine,” “module,” “control,” “generator,” or the like may also be used. As used in this description and in this case, these terms—whether expressed with or without a modifying clause—are also intended to be synonymous with the term “executable component,” and thus also have a structure that is well understood by those of ordinary skill in the art of computing.

In the description that follows, embodiments are described with reference to acts that are performed by one or more computing systems. If such acts are implemented in software, one or more processors (of the associated computing system that performs the act) direct the operation of the computing system in response to having executed computer-executable instructions that constitute an executable component. For example, such computer-executable instructions may be embodied on one or more computer-readable media that form a computer program product. An example of such an operation involves the manipulation of data.

The computer-executable instructions (and the manipulated data) may be stored in the memory 104 of the computing system 100. Computing system 100 may also contain communication channels 108 that allow the computing system 100 to communicate with other computing systems over, for example, network 110.

While not all computing systems require a user interface, in some embodiments the computing system 100 includes a user interface 112 for use in interfacing with a user. The user interface 112 may include output mechanisms 112A as well as input mechanisms 112B. The principles described herein are not limited to the precise output mechanisms 112A or input mechanisms 112B as such will depend on the nature of the device. However, output mechanisms 112A might include, for instance, speakers, displays, tactile output, holograms and so forth. Examples of input mechanisms 112B might include, for instance, microphones, touchscreens, holograms, cameras, keyboards, mouse or other pointer input, sensors of any type, and so forth.

Embodiments described herein may comprise or utilize a special purpose or general-purpose computing system including computer hardware, such as, for example, one or more processors and system memory, as discussed in greater detail below. Embodiments described herein also include physical and other computer-readable media for carrying or storing computer-executable instructions and/or data structures. Such computer-readable media can be any available media that can be accessed by a general purpose or special purpose computing system. Computer-readable media that store computer-executable instructions are physical storage media. Computer-readable media that carry computer-executable instructions are transmission media. Thus, by way of example embodiments of the present subject matter can comprise at least two distinctly different kinds of computer-readable media: storage media and transmission media.

Computer-readable storage media include RAM, ROM, EEPROM, solid state drives (“SSDs”), flash memory, phase-change memory (“PCM”), CD-ROM or other optical disk storage, magnetic disk storage or other magnetic storage devices, or any other physical and tangible storage medium which can be used to store desired program code in the form of computer-executable instructions or data structures and which can be accessed and executed by a general purpose or special purpose computing system to implement the disclosed functionality of the present disclosure.

A “network” refers to one or more data links that enable the transport of electronic data between computing systems and/or modules and/or other electronic devices. When information is transferred or provided over a network or another communications connection (either hardwired, wireless, or a combination of hardwired or wireless) to a computing system, the computing system properly views the connection as a transmission medium. Networks may be “private” or they may be “public,” or networks may share qualities of both private and public networks. A private network may be any network that has restricted access such that only the computer systems and/or modules and/or other electronic devices that are provided and/or permitted access to the private network may transport electronic data through the one or more data links that comprise the private network. A public network may, on the other hand, not restrict access and allow any computer systems and/or modules and/or other electronic devices capable of connecting to the network to use the one or more data links comprising the network to transport electronic data.

For example, a private network found within an organization, such as a private business, restricts transport of electronic data between only those computer systems and/or modules and/or other electronic devices within the organization. Conversely, the Internet is an example of a public network where access to the network is, generally, not restricted. Computer systems and/or modules and/or other electronic devices may often be connected simultaneously or serially to multiple networks, some of which may be private, some of which may be public, and some of which may be varying degrees of public and private. For example, a laptop computer may be permitted access to a closed network, such as a network for a private business that enables transport of electronic data between the computing systems of permitted business employees, and the same laptop computer may also access an open network, such as the Internet, at the same time or at a different time as it accesses the exemplary closed network.

Transmission media can include a network and/or data links which can be used to carry desired program code in the form of computer-executable instructions or data structures and which can be accessed and executed by a general purpose or special purpose computing system. Combinations of the above should also be included within the scope of computer-readable media.

Further, upon reaching various computing system components, program code in the form of computer-executable instructions or data structures can be transferred automatically from transmission media to storage media (or vice versa). For example, computer-executable instructions or data structures received over a network or data link can be buffered in RAM within a network interface module (e.g., a “NIC”) and then eventually transferred to computing system RAM and/or to less volatile storage media at a computing system. Thus, it should be understood that storage media can be included in computing system components that also—or even primarily—utilize transmission media.

Computer-executable instructions comprise, for example, instructions and data which, when executed at a processor, cause a general purpose computing system, special purpose computing system, or special purpose processing device to perform a certain function or group of functions. Alternatively, or additionally, the computer-executable instructions may configure the computing system to perform a certain function or group of functions. The computer executable instructions may be, for example, binaries or even instructions that undergo some translation (such as compilation) before direct execution by the processors, such as intermediate format instructions like assembly language, or even source code.

Those skilled in the art will appreciate that the subject matter disclosed herein may be practiced in network computing environments with many types of computing system configurations, including, personal computers, desktop computers, laptop computers, message processors, hand-held devices, multi-processor systems, microprocessor-based or programmable consumer electronics, network PCs, minicomputers, mainframe computers, tablets, mobile telephones, PDAs, pagers, routers, switches, datacenters, wearables (e.g., glasses) and the like. The subject matter disclosed herein may also be practiced in distributed system environments where local and remote computing systems, which are linked (either by hardwired data links, wireless data links, or by a combination of hardwired and wireless data links) through a network, both perform tasks. In a distributed system environment, program modules may be located in both local and remote memory storage devices.

Those skilled in the art will also appreciate that the implementations discussed herein may be practiced in a cloud computing environment. Cloud computing environments may be distributed, although this is not required. When distributed, cloud computing environments may be distributed internationally within an organization and/or have components possessed across multiple organizations. In this description and the following claims, “cloud computing” is defined as a model for enabling on-demand network access to a shared pool of configurable computing resources (e.g., networks, servers, storage, applications, and services). The definition of “cloud computing” is not limited to any of the other numerous advantages that can be obtained from such a model when properly deployed.

Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

The foregoing detailed description has set forth various implementations of the devices and/or processes via the use of block diagrams, schematics, and examples. Insofar as such block diagrams, schematics, and examples contain one or more functions and/or operations, it will be understood by those skilled in the art that each function and/or operation within such block diagrams, flowcharts, or examples can be implemented, individually and/or collectively, by a wide range of hardware, software, firmware, or virtually any combination thereof. In one implementation, the present subject matter may be implemented via Application Specific Integrated Circuits (ASICs). However, those skilled in the art will recognize that the implementations disclosed herein, in whole or in part, can be equivalently implemented in standard integrated circuits, as one or more computer programs running on one or more computers (e.g., as one or more programs running on one or more computer systems), as one or more programs running on one or more controllers (e.g., microcontrollers) as one or more programs running on one or more processors (e.g., microprocessors), as firmware, or as virtually any combination thereof, and that designing the circuitry and/or writing the code for the software and or firmware would be well within the skill of one of ordinary skill in the art in light of this disclosure.

Those of skill in the art will recognize that many of the methods or algorithms set out herein may employ additional acts, may omit some acts, and/or may execute acts in a different order than specified.

In addition, those skilled in the art will appreciate that the mechanisms taught herein are capable of being distributed as a program product in a variety of forms, and that an illustrative implementation applies equally regardless of the particular type of signal bearing media used to actually carry out the distribution. Examples of signal bearing media include, but are not limited to, the following: recordable type media such as floppy disks, hard disk drives, CD ROMs, digital tape, and computer memory.

Various embodiments of the disclosure are described herein. It will be recognized that features specified in each embodiment may be combined with other specified features to provide further embodiments of the present disclosure.

The following embodiments are included within the scope of the disclosure:

Embodiments of the present disclosure include a system, comprising: (A) one or more processors; and (B) one or more computer readable hardware storage devices that comprise computer executable instructions executable by at least one of the one or more processors to cause the computer system to: (1) identify a set of physical features from a dataset comprising: (a) physical measurements of a first plurality of bacterial strains in a plurality of microbiome samples from a plurality of subjects, respectively; (b) phenotypic data comprising a phenotypic feature for each subject of the plurality of subjects; and (c) physical measurements of a second plurality of bacterial strains; (2) quantify a frequency of each physical feature of the set of physical features; (3) determine an association value for each physical feature of the set of physical features with the phenotypic feature; (4) identify a subset of bacterial strains of the second plurality of bacterial strains that is associated with the phenotypic feature, the subset of bacterial strains comprising a first bacterial strain of the second plurality of bacterial strains; and (5) output the subset of bacterial strains.

In some embodiments, each physical feature of the set of physical features comprises a gene. In some embodiments, the physical measurements comprise sequencing information.

In some embodiments, the phenotypic feature is a BMI ranging from 18.5 to 24.9. In other embodiments, the phenotypic feature is a BMI of at least 25.0.

In some embodiments, the phenotypic feature is a diagnosis of colorectal cancer, Crohn's disease, irritable bowel syndrome, non-alcoholic fatty liver disease (NAFLD), nonalcoholic steatohepatitis (NASH), pancreatic cancer, liver cancer, or stomach cancer. In some embodiments, the phenotypic feature is a diagnosis of colorectal cancer. In some embodiments, the phenotypic feature is a diagnosis of Crohn's disease.

In some embodiments, the determining an association value comprises forming co-abundant gene groups (CAGs) by grouping genes by average linkage clustering. CAGs may correspond to the core genomes of species, combinations of co-abundant species, or accessory genomic elements such as transposons, bacteriophage, or so-called genome “islands” which vary across strains. In embodiments, a genomic island ranges from about 10 to about 35 kilobases in size. In some embodiments, the quantifying the frequency of each physical feature comprises aligning portions of the sequencing information and filtering to remove multi-mapping reads.

Further embodiments of the present disclosure include a system, comprising: (A) one or more processors; and (B) one or more computer readable hardware storage devices that comprise computer executable instructions executable by at least one of the one or more processors to cause the computer system to: (1) identify a set of physical features from physical measurements of a first bacterial strain; (2) determine an association value for each physical feature of the set of physical features with a plurality of phenotypic features by determining a frequency of each physical feature of the set of physical features in a dataset comprising; (a) physical measurements of a plurality of microbiome samples from a plurality of subjects, respectively; (b) phenotypic data comprising the phenotypic feature for each subject of the plurality of subjects; and (c) physical measurements of a plurality of bacterial strains; and (3) identify a phenotypic feature of the plurality of phenotypic features that is associated with the first bacterial strain based at least on the association value; and (4) output the phenotypic feature.

In some embodiments, each physical feature of the set of physical features comprises a gene. In some embodiments, the determining an association value comprises forming co-abundant gene groups (CAGs) by grouping genes by average linkage clustering. In some embodiments, the physical measurements comprise sequencing information. In some embodiments, the determining the frequency of each physical feature comprises aligning portions of the sequencing information and filtering to remove multi-mapping reads.

In some embodiments, the phenotypic feature is a BMI ranging from 18.5 to 24.9. In other embodiments, the phenotypic feature is a BMI of at least 25.0.

In some embodiments, the phenotypic feature is a diagnosis of colorectal cancer, Crohn's disease, irritable bowel syndrome, non-alcoholic fatty liver disease (NAFLD), nonalcoholic steatohepatitis (NASH), pancreatic cancer, liver cancer, or stomach cancer. In some embodiments, the phenotypic feature is a diagnosis of colorectal cancer. In some embodiments, the phenotypic feature is a diagnosis of Crohn's disease.

Additional embodiments of the disclosure include a system, comprising: (A) one or more processors; and (B) one or more computer readable hardware storage devices that comprise computer executable instructions executable by at least one of the one or more processors to cause the computer system to: (1) receive first physical measurements of a first plurality of bacterial strains in a microbiome sample from a subject; (2) identify a first set of physical features from the first physical measurements; (3) determine an association value for each physical feature of the first set of physical features with a plurality of phenotypic features, the association value being based on a frequency of a second set of physical features in a dataset comprising: (a) physical measurements of a second plurality of bacterial strains in a plurality of microbiome samples from a plurality of subjects, respectively; (b) phenotypic data comprising a phenotypic feature for each subject of the plurality of subjects; and (c) physical measurements of a third plurality of bacterial strains; (4) identify a phenotypic feature of the plurality of phenotypic features that is associated with the first plurality of bacterial strains based at least on the association value; and (5) output the phenotypic feature.

In some embodiments, each physical feature of the set of physical features comprises a gene. In some embodiments, the determining an association value comprises forming co-abundant gene groups (CAGs) by grouping genes by average linkage clustering. In some embodiments, the physical measurements comprise sequencing information. In some embodiments, the determining the frequency of each physical feature comprises aligning portions of the sequencing information and filtering to remove multi-mapping reads.

In some embodiments, the phenotypic feature is a BMI ranging from 18.5 to 24.9. In some embodiments, the phenotypic feature is a BMI of at least 25.0.

In some embodiments, the phenotypic feature is a diagnosis of colorectal cancer, Crohn's disease, irritable bowel syndrome, non-alcoholic fatty liver disease (NAFLD), nonalcoholic steatohepatitis (NASH), pancreatic cancer, liver cancer, or stomach cancer. In some embodiments, the phenotypic feature is a diagnosis of colorectal cancer. In some embodiments, the phenotypic feature is a diagnosis of Crohn's disease.

Further embodiments of the present disclosure include a method, comprising: (A) identifying a set of physical features from a dataset comprising: (1) physical measurements of a first plurality of bacterial strains in a plurality of microbiome samples from a plurality of subjects, respectively; (2) phenotypic data comprising a phenotypic feature for each subject of the plurality of subjects; and (3) physical measurements of a second plurality of bacterial strains; (B) quantifying a frequency of each physical feature of the set of physical features; (C) determining an association value for each physical feature of the set of physical features with the phenotypic feature; (D) identifying a subset of bacterial strains of the second plurality of bacterial strains that is associated with the phenotypic feature, the subset of bacterial strains comprising a first bacterial strain of the second plurality of bacterial strains; and (E) outputting the subset of bacterial strains.

In some embodiments, the determining an association value comprises forming co-abundant gene groups (CAGs) by grouping genes by average linkage clustering. In some embodiments, the physical measurements comprise sequencing information. In some embodiments, the determining the frequency of each physical feature comprises aligning portions of the sequencing information and filtering to remove multi-mapping reads.

Still further embodiments of the disclosure include a method, comprising: (A) identifying a first set of physical features from physical measurements of a first bacterial strain; (B) determining an association value for each physical feature of the first set of physical features with a plurality of phenotypic features by determining a frequency of each physical feature of the set of physical features in a dataset comprising; (1) physical measurements of a plurality of microbiome samples from a plurality of subjects, respectively; (2) phenotypic data comprising the phenotypic feature for each subject of the plurality of subjects; and (3) physical measurements of a plurality of bacterial strains; and (C) identifying a phenotypic feature of the plurality of phenotypic features that is associated with the first bacterial strain based at least on the association value; and (D) outputting the phenotypic feature.

In some embodiments, the determining an association value comprises forming co-abundant gene groups (CAGs) by grouping genes by average linkage clustering. In some embodiments, the physical measurements comprise sequencing information. In some embodiments, the determining the frequency of each physical feature comprises aligning portions of the sequencing information and filtering to remove multi-mapping reads.

In any of the above embodiments, similar processes can be used for second bacterial strains, and the like.

Yet further embodiments of the disclosure include a method, comprising: (A) receiving first physical measurements of a first plurality of bacterial strains in a microbiome sample from a subject; (B) identifying a first set of physical features from the first physical measurements; (C) determining an association value for each physical feature of the first set of physical features with a plurality of phenotypic features, the association value being based on a frequency of a second set of physical features in a dataset comprising: (1) physical measurements of a second plurality of bacterial strains in a plurality of microbiome samples from a plurality of subjects, respectively; (2) phenotypic data comprising a phenotypic feature for each subject of the plurality of subjects; and (3) physical measurements of a third plurality of bacterial strains; (D) identifying a phenotypic feature of the plurality of phenotypic features that is associated with the first plurality of bacterial strains based at least on the association value; and (E) outputting the phenotypic feature.

In some embodiments, the determining an association value comprises forming co-abundant gene groups (CAGs) by grouping genes by average linkage clustering. In some embodiments, the physical measurements comprise sequencing information. In some embodiments, the determining the frequency of each physical feature comprises aligning portions of the sequencing information and filtering to remove multi-mapping reads.

In some embodiments, the present methods are used to provide information about the likely response that a subject is to have to a particular treatment. For example, in embodiments, a phenotypic feature identified is a predicted response to a particular treatment regimen. In such embodiments, the methods of the present application may be used to identify such a phenotypic feature. Thus, in embodiments, the methods disclosed herein may be used to direct a clinical decision regarding whether a subject is to receive a specific treatment. In some embodiments, the present methods provide a high likelihood of response and may direct treatment, including aggressive treatment. In some embodiments, the present methods provide a low likelihood of response and may direct cessation of treatment, including aggressive treatment, and the use of palliative care, to avoid unnecessary toxicity from ineffective chemotherapies for a better quality of life. For example, if a particular subject diagnosed with a cancer, the present methods may be used to determine if the cancer is likely to be responsive to a particular treatment (e.g., ICI-based therapy). Accordingly, methods of the disclosure comprise a method for determining a subject's likelihood of response to a treatment regimen.

In embodiments, the methods described herein direct the selection of a therapeutic agent for treating a cancer in a subject. In certain embodiments, the methods described herein predict a subject's response to a treatment regimen comprising one or more therapeutic agents. In some embodiments, the methods described herein direct the selection of a therapeutic agent for treating a cancer in a subject. In embodiments, the methods described herein predict a subject's response to a treatment regimen comprising a combination of two or more therapeutic agents.

In embodiments, the therapeutic agent(s) comprise a chemotherapeutic agent. In various embodiments, the chemotherapeutic agent is selected from alkylating agents (e.g., thiotepa and CYTOXAN cyclosphosphamide); alkyl sulfonates (e.g., busulfan, improsulfan, and piposulfan); aziridines (e.g., benzodopa, carboquone, meturedopa, and uredopa); ethylenimines and methylamelamines (e.g., altretamine, triethylenemelamine, trietylenephosphoramide, triethiylenethiophosphoramide, and trimethylolomelamine); acetogenins (e.g., bullatacin and bullatacinone); camptothecin (e.g., topotecan); bryostatin; callystatin; CC-1065; cryptophycins (e.g., cryptophycin 1 and cryptophycin 8); dolastatin; duocarmycin; eleutherobin; pancratistatin; sarcodictyin; spongistatin; nitrogen mustards (e.g., chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard); nitroureas (e.g., carmustine, chlorozotocin, fotemustine, lomustine, nimustine, and ranimustine); antibiotics (e.g., enediyne antibiotics (e.g., calicheamicin, such as calicheamicin gammall and calicheamicin omegall)); dynemicin (e.g., dynemicin A); bisphosphonates (e.g., clodronate); an esperamicin; neocarzinostatin chromophore and related chromoprotein enediyne antiobiotic chromophores, aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, adriamycin doxorubicin (e.g., morpholino-doxorubicin, cyanomorpholino-doxorubicin, 2-pyrrolino-doxorubicin and deoxy doxorubicin), epirubicin, esorubicin, idarubicin, marcellomycin, mitomycins (e.g., mitomycin C), mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites (e.g., methotrexate and 5-fluorouracil (5-FU)); folic acid analogues such as denopterin, methotrexate, pteropterin, trimetrexate; purine analogs (e.g., fludarabine, 6-mercaptopurine, thiamiprine, and thioguanine); pyrimidine analogs (e.g., ancitabine, azacitidine, 6-azauridine, carmofur, cytarabine, dideoxyuridine, doxifluridine, enocitabine, and floxuridine); androgens (e.g., calusterone, dromostanolone propionate, epitiostanol, mepitiostane, and testolactone); anti-adrenals (e.g., minoglutethimide, mitotane, and trilostane); folic acid replenishers (e.g., folinic acid); aceglatone; aldophosphamide glycoside; aminolevulinic acid; eniluracil; amsacrine; bestrabucil; bisantrene; edatraxate; demecolcine; diaziquone; elformithine; elliptinium acetate; an epothilone; etoglucid; gallium nitrate; hydroxyurea; lentinan; lonidainine; maytansinoids (e.g., maytansine and ansamitocins); mitoguazone; mitoxantrone; mopidanmol; nitraerine; pentostatin; phenamet; pirarubicin; losoxantrone; podophyllinic acid; 2-ethylhydrazide; procarbazine; razoxane; rhizoxin; sizofuran; spirogermanium; tenuazonic acid; triaziquone; 2,2′,2″-trichlorotriethylamine; trichothecenes (e.g., T-2 toxin, verracurin A, roridin A, and anguidine); urethan; vindesine; dacarbazine; mannomustine; mitobronitol; mitolactol; pipobroman; gacytosine; arabinoside; cyclophosphamide; thiotepa; toxoids (e.g., TAXOL paclitaxel, ABRAXANE Cremophor-free, and TAXOTERE doxetaxel; chlorambucil; GEMZAR gemcitabine; 6-thioguanine; mercaptopurine; methotrexate; platinum analogs (e.g., cisplatin, oxaliplatin and carboplatin); vinblastine; platinum; etoposide (VP-16); ifosfamide; vincristine; NAVELBINE. vinorelbine; novantrone; teniposide; edatrexate; daunomycin; aminopterin; xeloda; ibandronate; irinotecan; topoisomerase inhibitors; difluoromethylornithine (DMFO); retinoids (e.g., retinoic acid); capecitabine; combretastatin; leucovorin (LV); oxaliplatin; lapatinib (Tykerb); inhibitors of PKC-1, Raf, H-Ras, EGFR (e.g., erlotinib) and VEGF-A, dacogen, velcade, and pharmaceutically acceptable salts, acids or derivatives of any of the agents listed herein.

In embodiments, the therapeutic agent used for ICI-based therapy is an immune checkpoint inhibitor (e.g., a PD-1 inhibitor, such as pembrolizumab or nivolumab; a PD-L 1 inhibitor, such as atezolizumab, avelumab, or durvalumab; a CTLA-4 inhibitor; a LAG-3 inhibitor; or a Tim-3 inhibitor). Other immune checkpoint inhibitors of interest include: PD-1 inhibitors, such as pembrolizumab (KEYTRUDA®), nivolumab (OPDIVO®), cemiplimab (LIBTAYO®), spartalizumab (PDR001), pidilizumab (CureTech), MEDI0680 (Medimmune), cemiplimab (REGN2810), dostarlimab (TSR-042), PF-06801591 (Pfizer), tislelizumab (BGB-A317), camrelizumab (INCSHR1210, SHR-1210), and AMP-224 (Amplimmune); PD-L 1 inhibitors, such as atezolizumab (TECENTRIQ®), avelumab (BAVENCIO®), durvalumab (IMFINZI®), FAZ053 (Novartis), and BMS-936559 (Bristol-Myers Squibb); and drugs that target CTLA-4, such as ipilimumab (YERVOY®). In some embodiments, the immune checkpoint inhibitor is a LAG-3 inhibitor (e.g., LAG525 (Novartis), BMS-986016 (Bristol-Myers Squibb), or TSR-033 (Tesaro)). In some embodiments, the immune checkpoint inhibitor is a TIM-3 inhibitor (e.g., MGB453 (Novartis) or TSR-022 (Tesaro)). In embodiments, the methods of treatment disclosed herein comprise administering an effective amount of a treatment regimen or a therapeutic agent to the subject. In various embodiments, effective amounts of a therapeutic agent can decrease the number of tumor cells, decrease the number of metastases, decrease tumor volume, induce apoptosis of cancer cells, induce cancer cell death, induce radio-sensitivity in cancer cells, inhibit angiogenesis near cancer cells, inhibit cancer cell proliferation, inhibit tumor growth, prevent metastasis, reduce the number of metastases, increase life expectancy, prolong a subject's life, reduce cancer-associated pain, and/or reduce relapse or re-occurrence of the cancer following treatment.

For administration, effective amounts (also referred to as doses) can be initially estimated based on results from in vitro assays and/or animal model studies. For example, a dose can be formulated in animal models to achieve a circulating concentration range that includes an IC50 as determined in cell culture against a particular target. Such information can be used to more accurately determine useful doses in subjects of interest.

The actual dose amount administered to a particular subject can be determined by a physician, veterinarian, or researcher taking into account parameters such as physical and physiological factors including target, body weight, severity of condition, type of cancer, previous or concurrent therapeutic interventions, idiopathy of the subject, and route of administration.

In embodiments, methods described herein include methods of treating a cancer in a subject in need thereof.

In any of the above embodiments, an additional treatment agent can be selected and optionally administered. Examples of such agents include one or more of anti-cancer drugs, therapy, surgery, adjuvant therapy, and neoadjuvant therapy, such as those specific agents described herein.

In one embodiment, the present methods further direct a clinical decision regarding whether a subject is to receive adjuvant therapy after primary, main, or initial treatment, including a single sole adjuvant therapy. Adjuvant therapy, also called adjuvant care, is treatment that is given in addition to the primary, main or initial treatment. By way of example, adjuvant therapy may be an additional treatment usually given after surgery where all detectable disease has been removed, but where there remains a statistical risk of relapse due to occult disease.

In some embodiments, the present methods direct a subject's treatment to include adjuvant therapy. For example, a subject that is scored to be responsive to a specific treatment may receive such treatment as adjuvant therapy. Further, the present methods may direct the identity of an adjuvant therapy. In one embodiment, the present methods may indicate that a subject will not be or will be less responsive to a specific treatment and therefore such a subject may not receive such treatment as adjuvant therapy. Accordingly, in some embodiments, the present methods provide for providing or withholding adjuvant therapy according to a subject's likely response. In this way, a subject's quality of life, and the cost of care, may be improved.

Embodiments of the present disclosure comprise a system, comprising:

one or more processors; and

one or more computer readable hardware storage devices that comprise computer executable instructions executable by at least one of the one or more processors to cause the computer system to:
identify a set of physical features from a dataset comprising:
   physical measurements of a first plurality of bacterial strains in a plurality of microbiome samples from a plurality of subjects, respectively;
   phenotypic data comprising a phenotypic feature for each subject of the plurality of subjects; and
   physical measurements of a second plurality of bacterial strains;
determine a frequency of each physical feature of the set of physical features;
determine an association value for each physical feature of the set of physical features with the phenotypic feature;
identify a subset of bacterial strains of the second plurality of bacterial strains that is associated with the phenotypic feature, the subset of bacterial strains comprising a first bacterial strain of the second plurality of bacterial strains; and
output the subset of bacterial strains.

In embodiments, the determining the frequency of each physical feature comprises aligning portions of the sequencing information and filtering to remove multi-mapping reads.

Embodiments of the present disclosure further comprise a system, comprising:

one or more processors; and

one or more computer readable hardware storage devices that comprise computer executable instructions executable by at least one of the one or more processors to cause the computer system to:
identify a set of physical features from physical measurements of a first bacterial strain;
determine an association value for each physical feature of the set of physical features with a plurality of phenotypic features by determining a frequency of each physical feature of the set of physical features in a dataset comprising;
   physical measurements of a plurality of microbiome samples from a plurality of subjects, respectively;
   phenotypic data comprising the phenotypic feature for each subject of the plurality of subjects; and
   physical measurements of a plurality of bacterial strains; and
identify a phenotypic feature of the plurality of phenotypic features that is associated with the first bacterial strain based at least on the association value; and
output the phenotypic feature.

In embodiments, the determining the frequency of each physical feature comprises aligning portions of the sequencing information and filtering to remove multi-mapping reads.

Embodiments of the present disclosure further comprise a system, comprising:

one or more processors; and

one or more computer readable hardware storage devices that comprise computer executable instructions executable by at least one of the one or more processors to cause the computer system to:
receive first physical measurements of a first plurality of bacterial strains in a microbiome sample from a subject;
identify a first set of physical features from the first physical measurements;
determine an association value for each physical feature of the first set of physical features with a plurality of phenotypic features, the association value being based on a frequency of a second set of physical features in a dataset comprising:
   physical measurements of a second plurality of bacterial strains in a plurality of microbiome samples from a plurality of subjects, respectively;
   phenotypic data comprising a phenotypic feature for each subject of the plurality of subjects; and
   physical measurements of a third plurality of bacterial strains;
identify a phenotypic feature of the plurality of phenotypic features that is associated with the first plurality of bacterial strains based at least on the association value; and
output the phenotypic feature.

In embodiments, the determining an association value comprises forming co-abundant gene groups (CAGs) by grouping genes by average linkage clustering. In embodiments, each physical feature of the set of physical features comprises a gene. In embodiments, each physical feature of the set of physical features comprises a genomic island. In embodiments, the genomic island ranges from about 10 to about 35 kilobases in size. In embodiments, the physical measurements comprise sequencing information.

In embodiments, the phenotypic feature is a BMI ranging from 18.5 to 24.9. In embodiments, the phenotypic feature is a BMI of at least 25.0. In embodiments, the phenotypic feature is a cancer. In embodiments, the cancer is responsive to a treatment regimen. In embodiments, the treatment regimen comprises immune checkpoint inhibitor (ICI)-based therapy. In embodiments, the cancer is a hematologic cancer. In embodiments, the cancer is a solid tumor. In embodiments, the cancer is melanoma. In embodiments, the phenotypic feature is colorectal cancer, Crohn's disease, irritable bowel syndrome, non-alcoholic fatty liver disease (NAFLD), nonalcoholic steatohepatitis (NASH), pancreatic cancer, liver cancer, or stomach cancer. In embodiments, the phenotypic feature is colorectal cancer. In embodiments, the phenotypic feature is Crohn's disease.

Embodiments of the present disclosure further comprise a method, comprising:

identifying a set of physical features from a dataset comprising:
physical measurements of a first plurality of bacterial strains in a plurality of microbiome samples from a plurality of subjects, respectively;
phenotypic data comprising a phenotypic feature for each subject of the plurality of subjects; and
physical measurements of a second plurality of bacterial strains;

quantifying a frequency of each physical feature of the set of physical features;

determining an association value for each physical feature of the set of physical features with the phenotypic feature;

identifying a subset of bacterial strains of the second plurality of bacterial strains that is associated with the phenotypic feature, the subset of bacterial strains comprising a first bacterial strain of the second plurality of bacterial strains; and

outputting the subset of bacterial strains.

Embodiments of the present disclosure further comprise a method, comprising:

identifying a first set of physical features from physical measurements of a first bacterial strain;

determining an association value for each physical feature of the first set of physical features with a plurality of phenotypic features by determining a frequency of each physical feature of the set of physical features in a dataset comprising;
physical measurements of a plurality of microbiome samples from a plurality of subjects, respectively;
phenotypic data comprising the phenotypic feature for each subject of the plurality of subjects; and
physical measurements of a plurality of bacterial strains; and

identifying a phenotypic feature of the plurality of phenotypic features that is associated with the first bacterial strain based at least on the association value; and

outputting the phenotypic feature.

Embodiments of the present disclosure further comprise a method, comprising:

identifying a set of physical features from a dataset comprising:
physical measurements of a first plurality of bacterial strains in a plurality of microbiome samples from a plurality of subjects, respectively;
phenotypic data comprising a phenotypic feature for each subject of the plurality of subjects; and
physical measurements of a second plurality of bacterial strains;

determining a frequency of each physical feature of the set of physical features;

comparing the physical features to a reference database;

determining an association value for each physical feature of the set of physical features with the phenotypic feature; and

identifying genomic islands that contain the physical features.

In embodiments, the determining the frequency of each physical feature comprises aligning portions of the sequencing information and filtering to remove multi-mapping reads.

Embodiments of the present disclosure further comprise a method, comprising:

receiving first physical measurements of a first plurality of bacterial strains in a microbiome sample from a subject;

identifying a first set of physical features from the first physical measurements;

determining an association value for each physical feature of the first set of physical features with a plurality of phenotypic features, the association value being based on a frequency of a second set of physical features in a dataset comprising:
physical measurements of a second plurality of bacterial strains in a plurality of microbiome samples from a plurality of subjects, respectively;
phenotypic data comprising a phenotypic feature for each subject of the plurality of subjects; and
physical measurements of a third plurality of bacterial strains;

identifying a phenotypic feature of the plurality of phenotypic features that is associated with the first plurality of bacterial strains based at least on the association value; and

outputting the phenotypic feature.

In embodiments, the method further comprises determining the frequency of each physical feature comprising aligning portions of the sequencing information and filtering to remove multi-mapping reads. In embodiments, the determining an association value comprises forming co-abundant gene groups (CAGs) by grouping genes by average linkage clustering.

In embodiments, each physical feature of the set of physical features comprises a gene. In embodiments, each physical feature of the set of physical features comprises a genomic island.

In embodiments, the genomic island ranges from about 10 to about 35 kilobases in size. In embodiments, the physical measurements comprise sequencing information.

In embodiments, the phenotypic feature is a BMI ranging from 18.5 to 24.9. In embodiments, the phenotypic feature is a BMI of at least 25.0.

In embodiments, the phenotypic feature is a cancer. In embodiments, the cancer is responsive to a treatment regimen. In embodiments, the treatment regimen comprises immune checkpoint inhibitor (ICI)-based therapy. In embodiments, the cancer is a hematologic cancer. In embodiments, the cancer is a solid tumor. In embodiments, the cancer is melanoma. In embodiments, the phenotypic feature is colorectal cancer, Crohn's disease, irritable bowel syndrome, non-alcoholic fatty liver disease (NAFLD), nonalcoholic steatohepatitis (NASH), pancreatic cancer, liver cancer, or stomach cancer. In embodiments, the phenotypic feature is colorectal cancer. In embodiments, the phenotypic feature is Crohn's disease.

Embodiments of the present disclosure further comprise a method of treating a disease in a subject with a treatment regimen based on the presence of the subset of bacterial strains in a sample from the subject, the subset of bacterial strains being identified by the method described herein.

Embodiments of the present disclosure further comprise a method of treatment of a disease in a subject in need thereof, the method comprising:

administering a treatment regimen to the subject, wherein the subset of bacterial strains is found in a sample from the subject.

In embodiments, the subset of bacterial strains is identified by the method described herein.

In embodiments, the disease is a cancer. In embodiments, the cancer is a hematologic cancer. In embodiments, the cancer is a solid tumor. In embodiments, the cancer is melanoma. In embodiments, the disease is colorectal cancer, Crohn's disease, irritable bowel syndrome, non-alcoholic fatty liver disease (NAFLD), nonalcoholic steatohepatitis (NASH), pancreatic cancer, liver cancer, or stomach cancer. In embodiments, the disease is colorectal cancer. In embodiments, the disease is Crohn's disease. In embodiments, the treatment regimen comprises immune checkpoint inhibitor (ICI)-based therapy.

Embodiments of this disclosure are further illustrated by the following examples.

### EXAMPLES

### Example 1

Association of Crohn's Disease and E. coli SF-166 (NCBI GCF_001280385.1)

Input data consists of a microbiome dataset generated from stool samples that were obtained from subjects both with and without a diagnosis of Crohn's disease. The stool samples were processed with Whole-Genome Shotgun sequencing on the Illumina platform. In step 1, the raw reads were aligned against a database of reference genes using DIAMOND. In step 2, the aligned reads were de-duplicated using FAMLI to identify the single most likely gene of origin for each genomic fragment. In step 3, the abundance of each gene was grouped by co-abundant gene groups (CAGs), which were generated previously by average linkage clustering with a cosine distance threshold of 0.25. In step 4, the abundance of each individual CAG was tested for association with IBD phenotype using the corncob statistical test. In step 5, a collection of bacterial isolate genomes were aligned against the gene catalog used in step 1. In step 6, each bacterial isolate genome was summarized on the basis of the estimated coefficient of association for each CAG, using those genes from each CAG, which align to each isolate genome. In step 7, bacterial isolates were identified (using the data from step 6), which contain the largest proportion of genes which belong to a CAG with an estimated coefficient of association with Crohn's disease with a significant p-value. In step 8, each bacterial genome was summarized as a map containing the physical location of each gene that is significantly associated with the Crohn's disease phenotype.

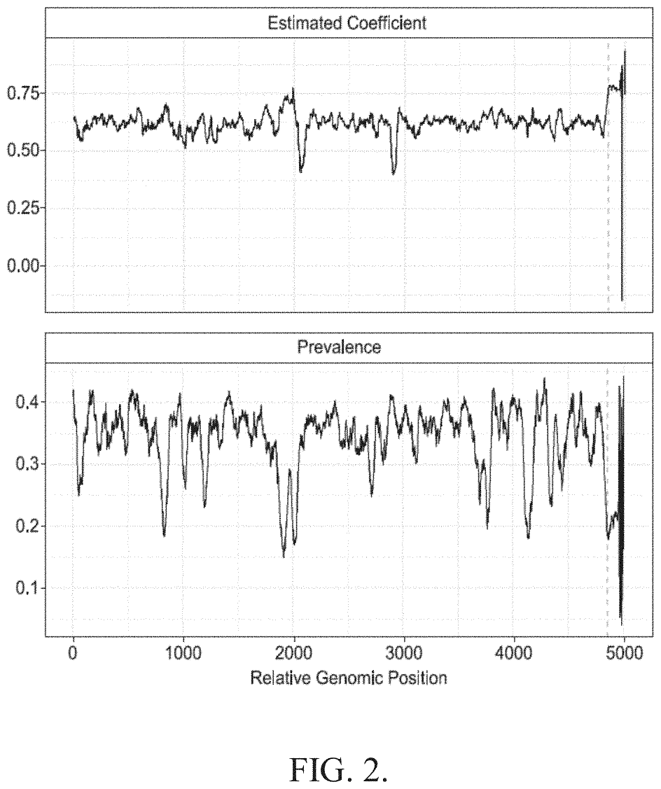

An example of a genome map produced using the methods as described herein is shown in FIG. 2. The top panel of FIG. 2 shows the estimated coefficient associating the genes present in the E. coli genome assembly GCF 001280385.1 with Crohn's Disease (CD) as assessed in a published cohort of subjects. A higher estimated coefficient indicates that those genes are more abundant in the gut microbiome of participants with CD, compared with the gut microbiome of those participants who have not been diagnosed with CD. The lower panel of FIG. 2 shows the prevalence of every gene, which is a measure of the proportion of gut microbiome samples across the entire cohort for which each gene was detected as being present. The vertical dashed line marks the boundary of the bacterial chromosome with a plasmid contained in that genome. As can be seen, the plasmid is less prevalent than the core genome, meaning that not all E. coli contain this plasmid. Importantly, the estimated coefficient of association is higher in the plasmid than in the main chromosome. In other words, although the plasmid is less commonly found in E. coli, it is more strongly associated with CD.

### Example 2

Colorectal Cancer and Fusobacterium nucleatum Subsp. Nucleatum ATCC 23726

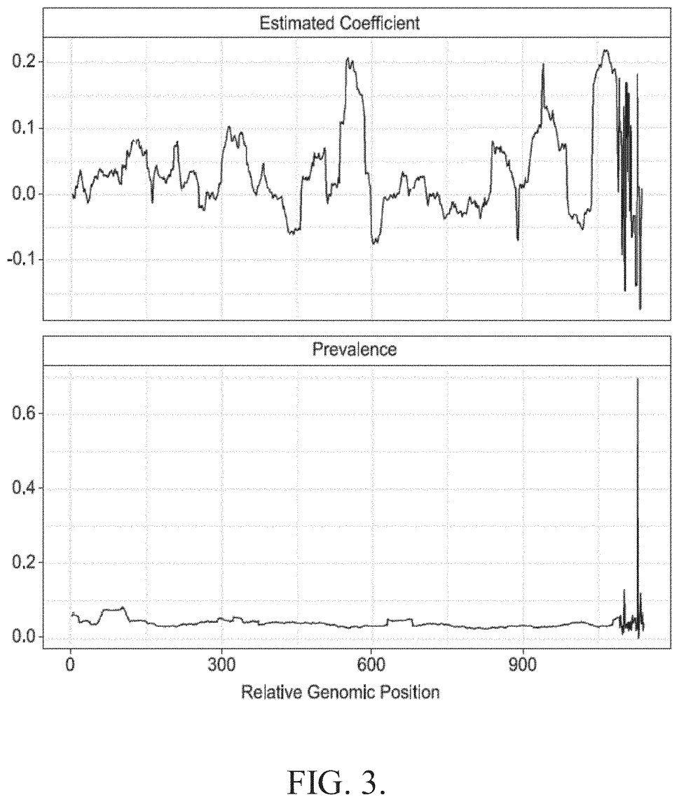

An example of a genome map produced using the methods as described herein is shown in FIG. 3. The top panel of FIG. 3 shows the estimated coefficient associating the genes present in the Fusobacterium nucleatum subsp. nucleatum ATCC 23726 genome with colorectal cancer (CRC). While F. nucleatum has been found to be present in colonic and metastatic tumors, the presence of the F. nucleatum core genome is not strongly associated with CRC (as can be seen in the top panel, the scale bar is centered on 0). However, three regions of the genome appear to be strongly positively associated with CRC.

This observation suggests that there may be other microbes present that are closely related to Fusobacterium nucleatum subsp. nucleatum ATCC 23726 and share genes with the core genome. The presence of these related microbes generally has no impact on the development of CRC. However, when a microbe is present in a subject's microbiome and contains any of the three genomic regions, that subject has a higher chance of developing CRC.

### Example 3

### Geneshot for Identification of Genome Islands Associated with Immunotherapy Response in Subjects with Metastatic Melanoma Analysis of Microbiome Surveys

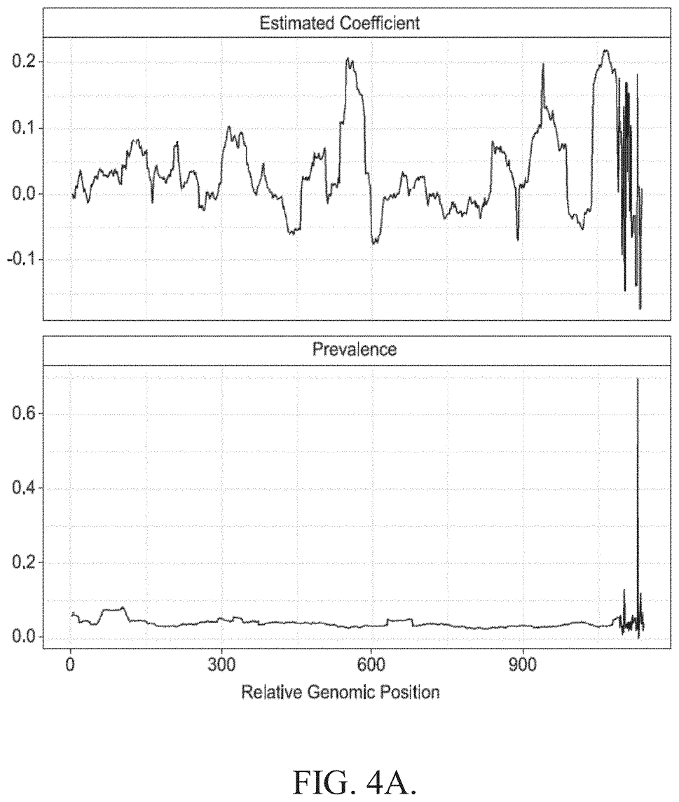

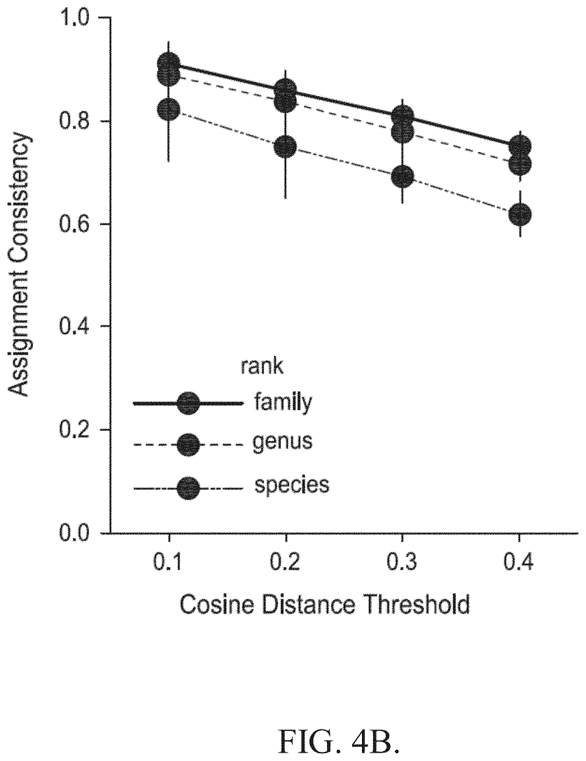

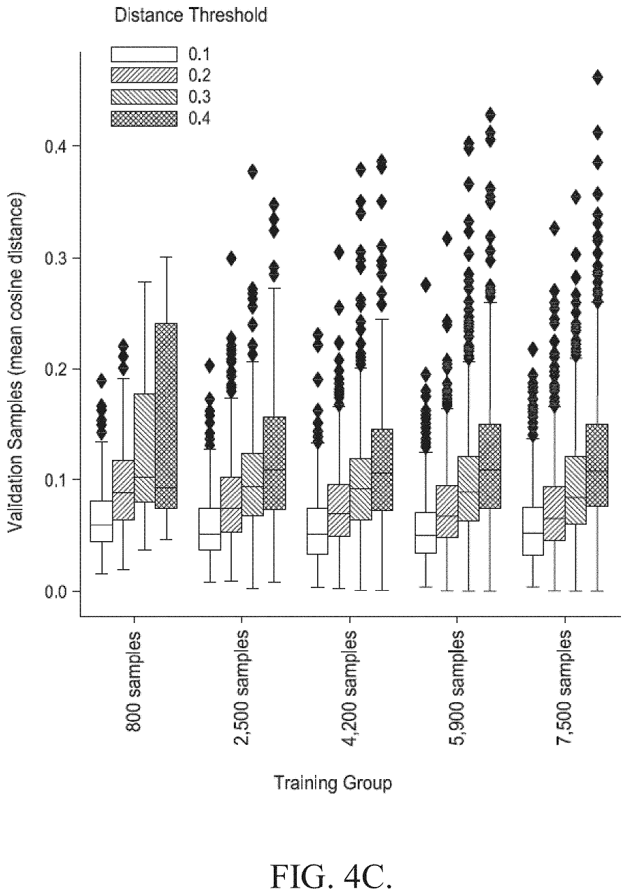

This example describes an analytical approach used for gene-level metagenomic analysis across multiple microbiome association studies. First, microbial genes were identified by de novo assembly of 12,667 host-associated metagenomes and the abundance of each gene is estimated by alignment of unassembled reads from each sample against that gene catalog. Second, different models were tested for the identification of co-abundant gene groups (CAGs) which independently vary the amino acid identity used for clustering genes, the number of samples used for model training, and the cosine similarity threshold. Third, a single model was selected for CAG construction on the basis of consistent CAG membership, robust co-abundance in held-out validation samples, and consistent taxonomic annotation within clusters. Fourth, the CAGalog v1.0 was assembled from the model selected, including the taxonomic and functional predictions for each individual gene. Finally, new metagenomic datasets from microbiome surveys were analyzed with the geneshot analysis pipeline and the CAGalog reference database, generating a relative abundance measure for each gene across every sample, and testing for association with health measures using the corncob statistical package. The results are shown in FIG. 4-FIG. 8.

FIGS. 4A-4C show co-abundant gene group (CAG) characteristics across a range of independent parameters selected. FIG. 4A shows the distribution of CAG sizes as represented by the number of genes (vertical axis) that were grouped into CAGs binned by CAG size (horizontal axis). FIG. 4B shows the consistency of taxonomic assignment as quantified as the proportion of genes in each CAG belonging to the consensus taxon (vertical axis) as a function of cosine distance threshold used for CAG construction (horizontal axis). FIG. 4C shows the distribution of cosine distances observed within each CAG in the validation dataset (vertical axis) as a function of training dataset size (horizontal axis) and cosine distance threshold (color scale).

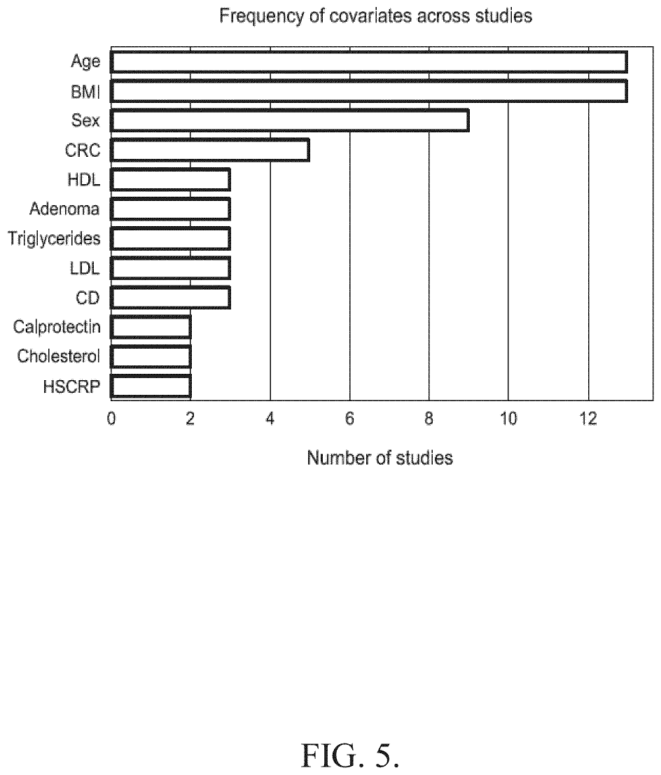

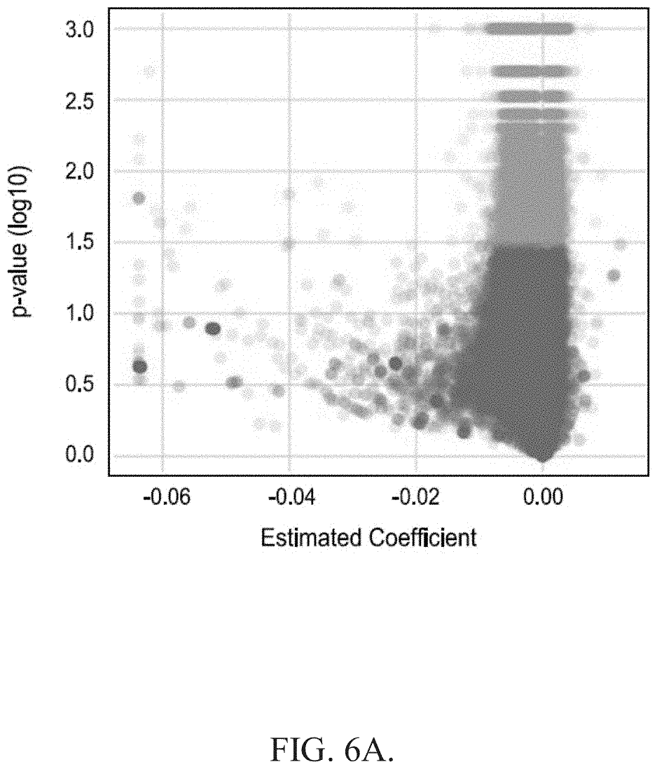

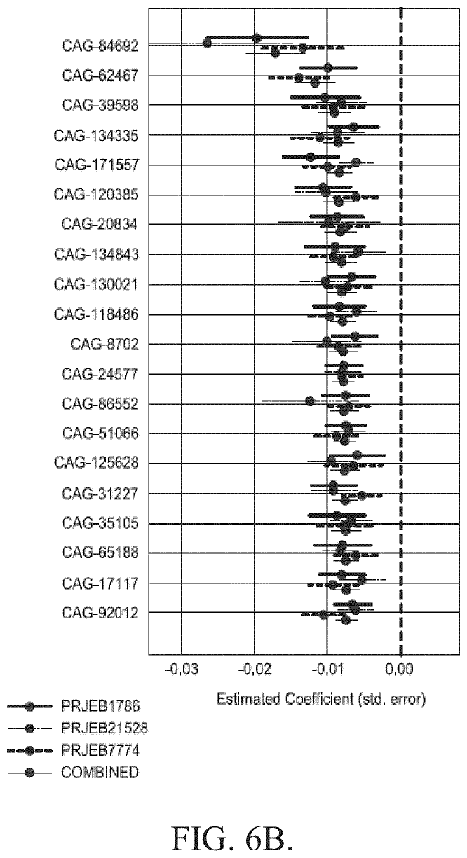

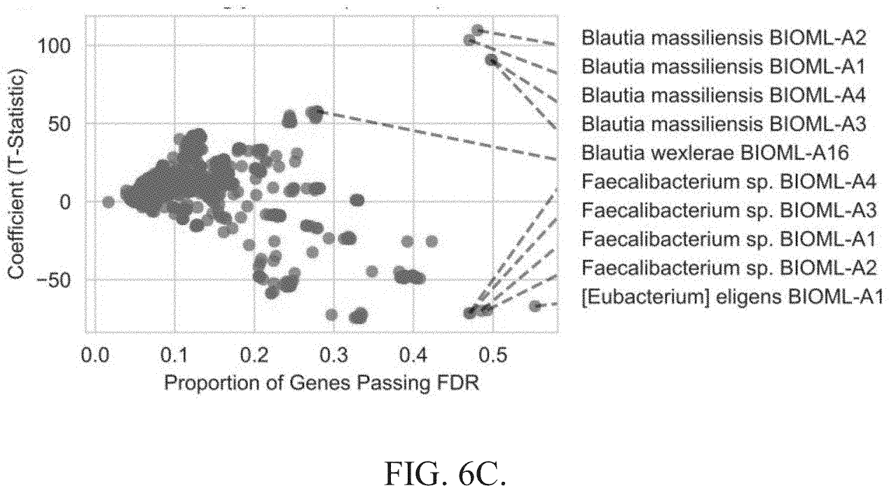

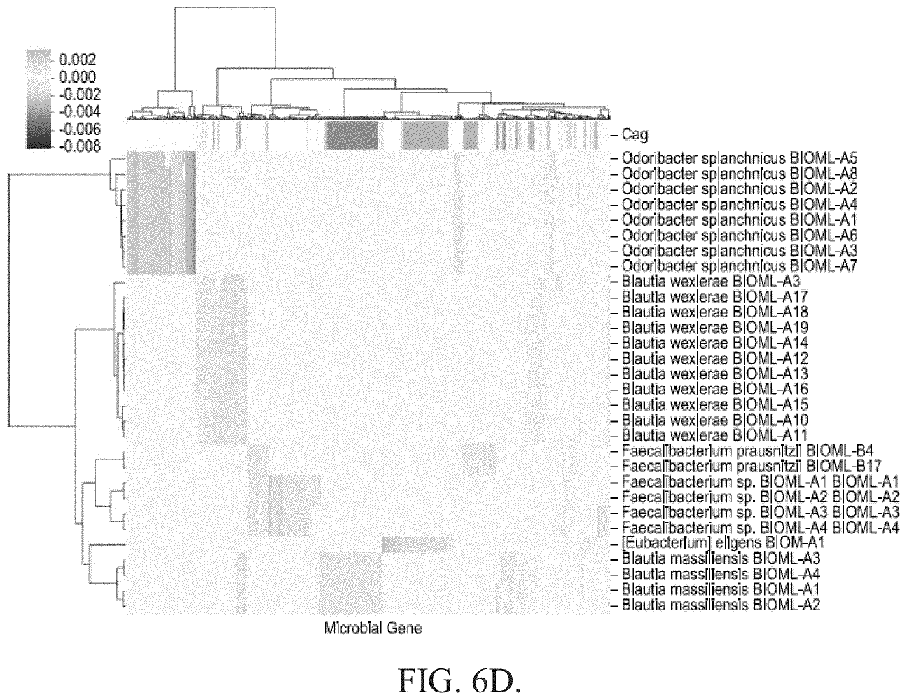

FIG. 5 shows the number of studies in which the same health measure or clinical covariate was measured. FIGS. 6A-6D show an example of meta-analysis for gene-level association of the microbiome with serum triglyceride levels. A volcano plot showing the p-value (log 10, vertical axis) and estimated coefficient (horizontal axis) for each microbial gene group with triglyceride levels is shown in FIG. 6A. FIG. 6B shows an example of meta-analysis combining the results from multiple studies for each microbial gene group, with the estimated coefficient and standard error (horizontal axis) shown for multiple gene groups (vertical axis) across multiple published studies (color facet) as well as the combined estimate generated from the meta-analysis. FIG. 6C shows a summary of genome-level associations using the BIO-ML collection on the basis of the proportion of genes from each genome which belong to a gene group which passes the 20% FDR threshold (horizontal axis) and the t-statistic summarizing the consistency of the positive or negative values of those estimated coefficients (vertical axis). A heatmap showing the triglyceride-associated genes found in each genome is depicted in FIG. 6D, with rows indicating bacterial isolates, columns indicating microbial genes, the color scale indicating the estimated coefficient for each gene passing 20% FDR, and the membership for the most abundant CAGs shown above the heatmap.

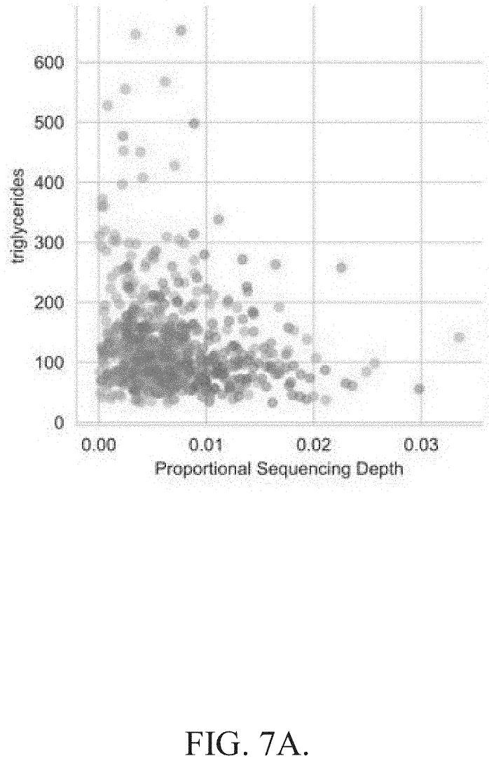

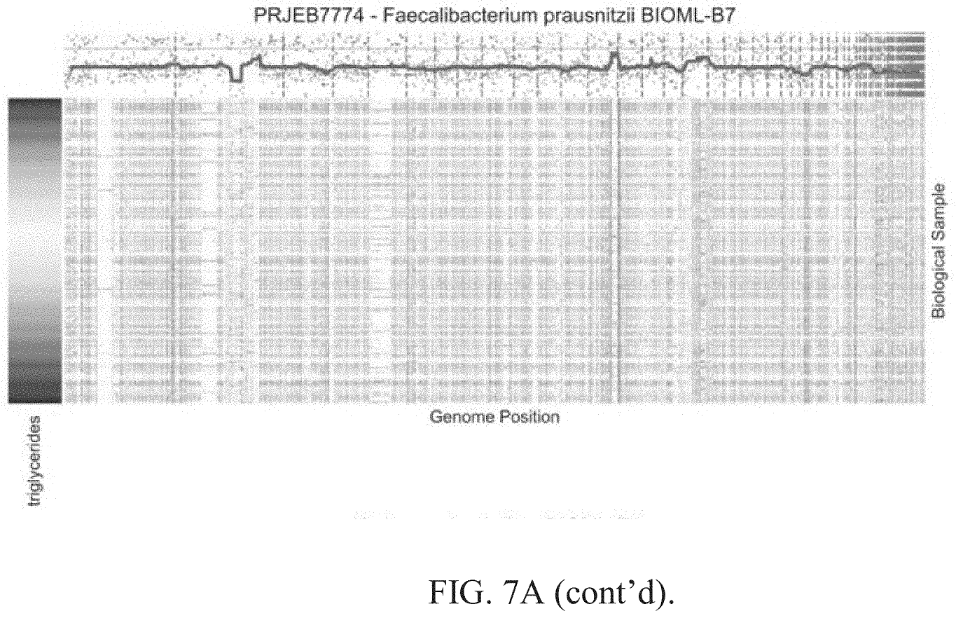

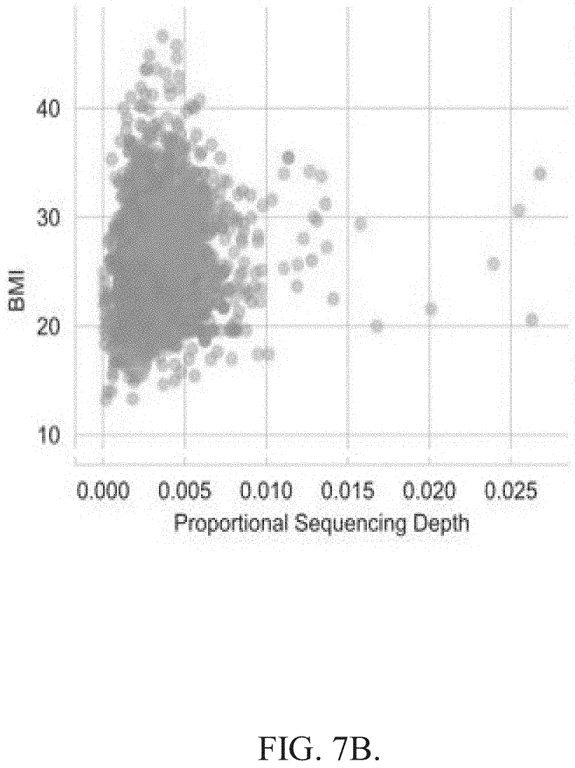

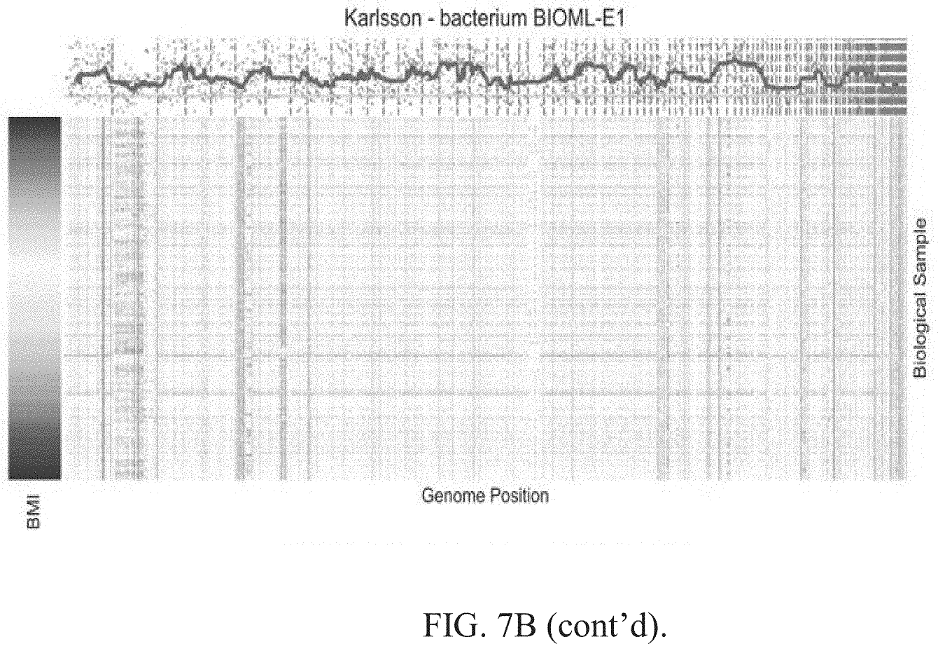

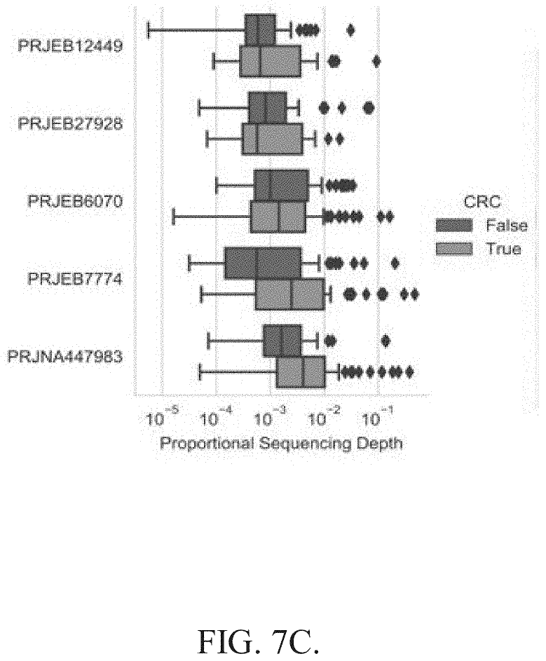

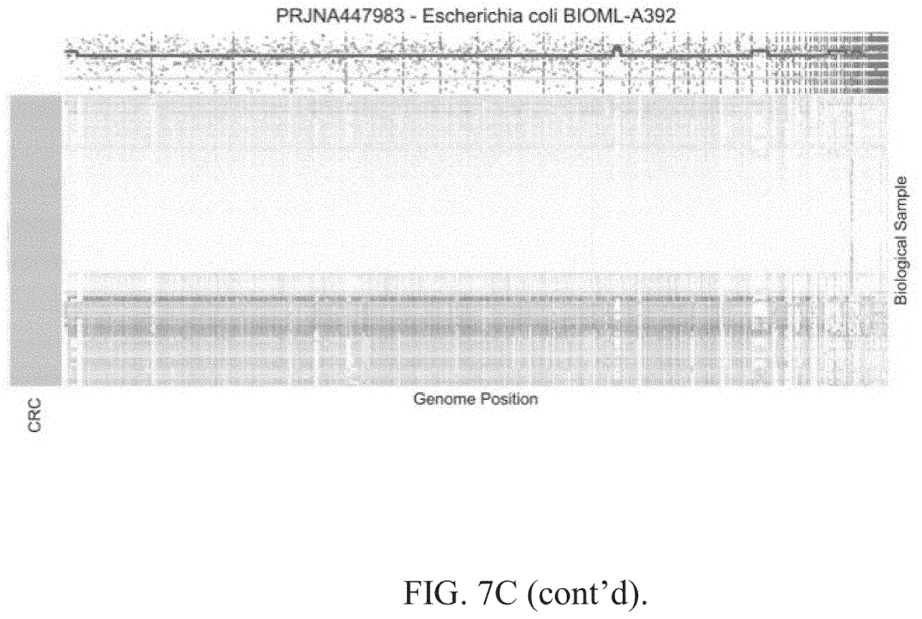

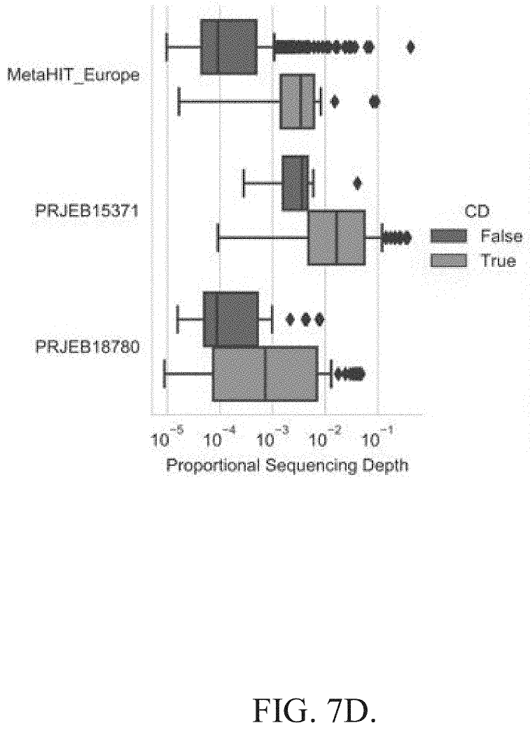

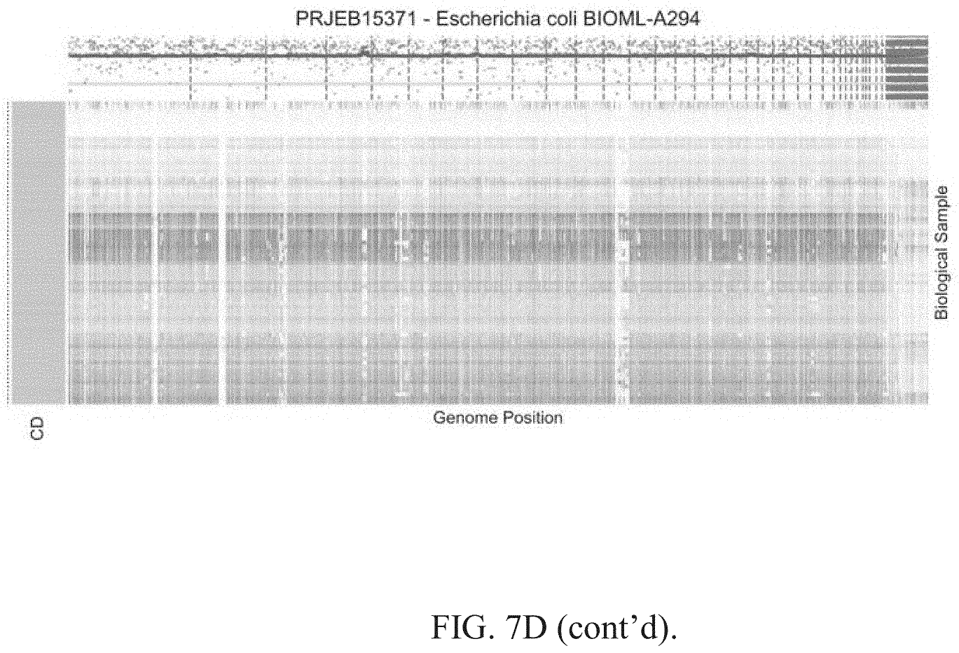

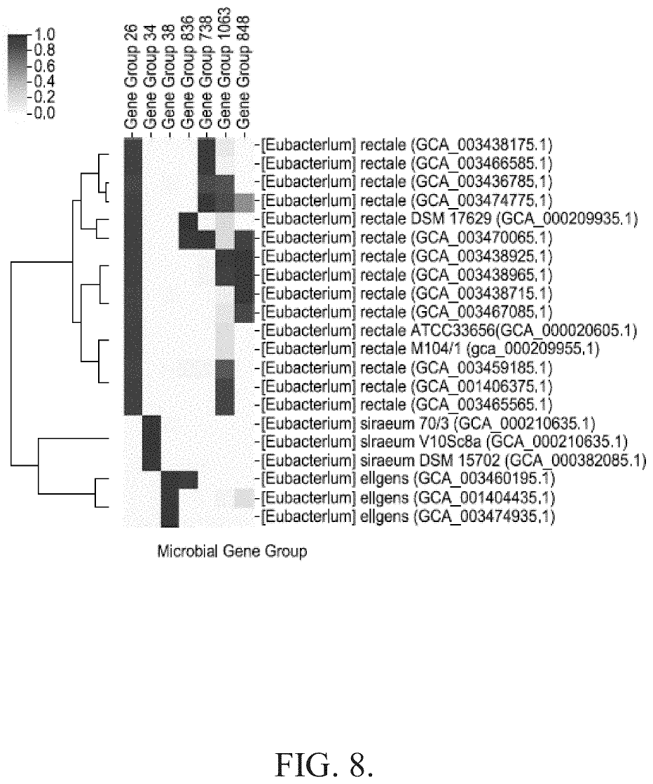

FIGS. 7A-7D show strain-level association of microbial isolates with human health. Selected examples of associations with triglyceride levels (FIG. 7A), BMI (FIG. 7B), colorectal cancer (FIG. 7C), and Crohn's disease (FIG. 7D). For each covariate, (left) the abundance of the indicated genome is summarized by the proportion of reads from each sample that align as a function of the participant's health measure, and (right) the abundance of each gene is plotted along the genome (horizontal axis) with the health measure shown in the left margin. Above each heatmap is plot of the estimated coefficient for each gene, with a blue trend line and an orange line indicating zero. FIG. 8 demonstrates how CAGs can be shared across species, strains, or horizontally transferred across species.

Software and Data Availability

All of the analytical steps which were carried out were executed from either (1) a Nextflow workflow or (2) a Jupyter notebook. Nextflow was used for compute-intensive processes which required Docker containerization, high-performance computing, or parallelization across tasks. Jupyter was used for interactive computing and summary analysis which resulted in the generation of figures in the final stages of analysis.

All Nextflow workflows indicate the Docker image used to execute each individual step as well as the computational resources that were used during the course of this project, which should enable any other researcher to execute the identical process on equivalent datasets in the future.

### CAGalog Database Development

De Novo Gene Catalog: The complete set of de novo assembled host-associated metagenomes available on MGnify (EMBL-EBI, Cambridgeshire, UK) as downloaded and deduplicated with MMseqs2, and filtered for prevalence according to the number of unique biological samples in which each deduplicated sample was found. While the workflow performs deduplication and prevalence filtering at a range of thresholds, 90% identity threshold for deduplication and a minimum prevalence of 5 biological samples was selected to generate the gene catalog used to build the CAGalog.

Co-Abundance Clustering: The de novo gene catalog was clustered by co-abundance across a set of 12,667 microbiome samples. This workflow was responsible for (a) downloading FASTQ data for published microbiome samples, (b) aligning WGS data against the deduplicated gene catalog, (c) filtering raw alignments with the FAMLI algorithm to ensure a unique assignment for each query, (d) clustering reference sequences by amino acid identity thresholds, and (e) identifying co-abundant gene groups (CAGs) using the Approximate Nearest Neighbor-based complete linkage clustering algorithm. The workflow generates independent results using all possible permutations of values for amino acid identity (50%, 60%, 70%, 80%, and 90%), training set size (800, 2,500, 4,200, 5,900, or 7,500 samples), and cosine distance threshold (0.1, 0.2, 0.3, and 0.4).

Formatting the CAGalog Reference Database: The CAGalog database HDF5 was created by combining the taxonomic and functional (eggNOG v4.5) annotation of each reference sequence into a single HDF5 for efficient parsing and retrieval.

Alignment to Genome Collections: The CAGalog reference database was aligned against reference genome collections performed with the DIAMOND aligner in translated BLASTX mode using the range culling feature to filter results to those which account for at least 50% of the length of the query gene sequence, while limiting to those alignments which score within a 1% margin of the top-scoring alignment for a given genomic region. The workflow also aggregates and reformats alignments into TSV and HDF5 format to produce the alignment summary files which were used to generate FIG. 4-FIG. 8.

Analyzing Published Microbiome Studies: Each published microbiome study was analyzed against the CAGalog reference database using the geneshot workflow, which encompasses (a) downloading WGS data in FASTQ format from SRA; (b) aligning WGS reads against the CAGalog gene catalog with DIAMOND; (c) filtering raw alignments with FAMLI, (d) summarizing the abundance of every gene across every sample on the basis of depth, coverage, and number of reads; and (e) assembling all result metrics into a single HDF5 file which includes abundance estimates for CAGs and KEGG Orthology groups (KOs).

Associating Health Measures with Gene Abundance: For each published microbiome study, the association of each annotated health features, was analyzed using the corncob algorithm. To facilitate comparison across studies with incompletely overlapping sets of health measurements, the marginal association of each individual health measure was determined. The output of this workflow is a CSV containing the estimated coefficient, standard error, and p-value for each gene group (CAG and KO) independently for each health measure.

Meta-Analysis of Microbiome Association: The corncob results were combined across studies for each health covariate using a workflow that uses the ‘betta’ function from the ‘breakaway’ R package. All results were provided as tabular text files including the summary metrics for every gene group as well as the association metrics estimated from each study individually.

### Example 4

### Geneshot for Identification of Genome Islands

Microbial genomic islands associated with a particular phenotypic feature are identified using geneshot, a bioinformatics tool for identifying testable hypotheses based on gene-level metagenomic analysis of WGS microbiome data. The bioinformatics tool described here is used for identifying testable hypotheses based on gene-level metagenomic analysis of WGS microbiome data (geneshot).

By applying geneshot to previously published cohorts, microbial genomic islands consistently associated with the particular phenotypic feature are identified. These results, as well as the underlying methodology, inform further mechanistic studies and facilitate the development of microbiome-enhanced therapeutics.

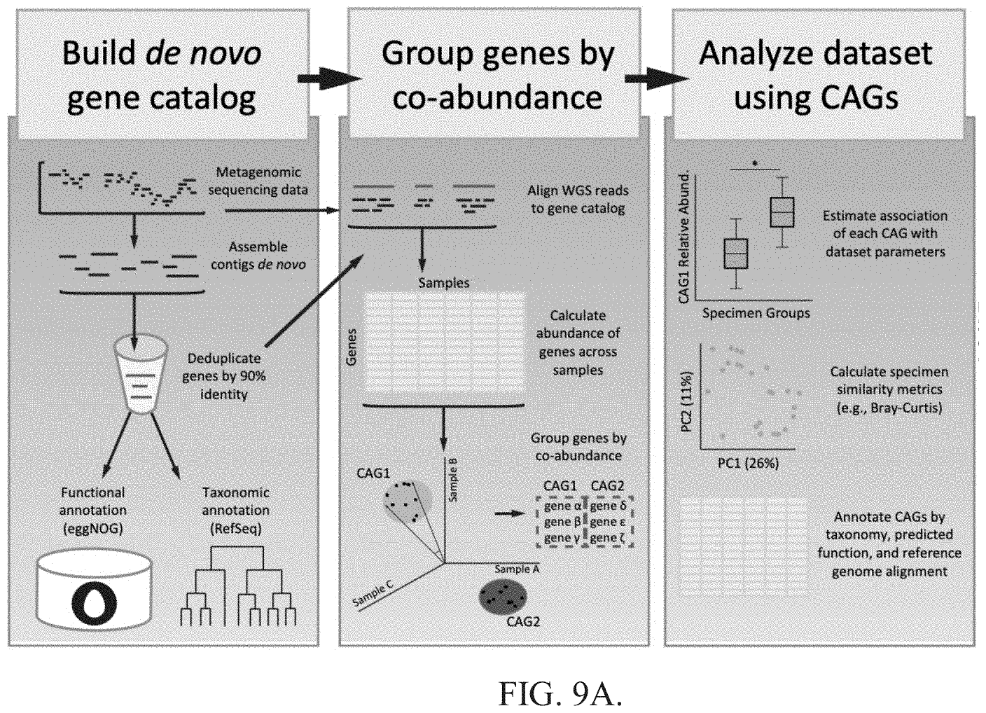

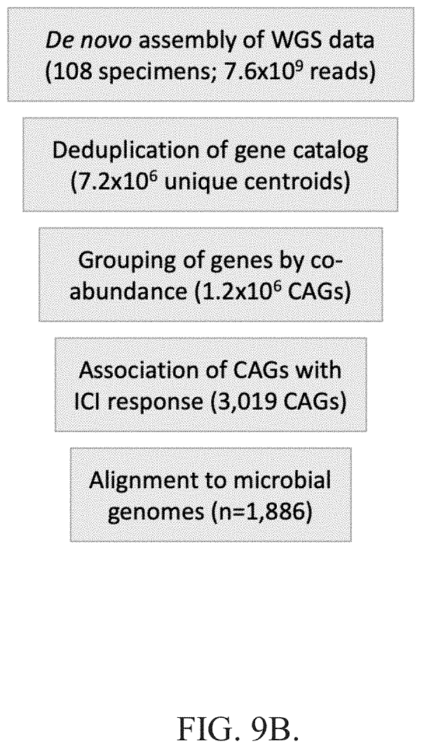

Protein-coding microbial genes are identified and combined into CAGs and input into a computational analysis pipeline for gene-level metagenomic analysis called geneshot. Geneshot is an end-to-end analysis workflow for microbiome experiments using CAGs as the fundamental unit of analysis. In brief, WGS data from each specimen are preprocessed to remove human reads and assemble de novo; predicted protein-coding genes are then deduplicated to make a reference gene catalog; the abundance of each gene in each specimen is estimated by assignment of raw reads against that gene catalog after rigorous handling of multiply-mapping reads; genes are grouped into CAGs based on their co-abundance observed across the samples; the possible taxonomic origins are identified (by alignment against RefSeq) and functionally (using eggNOG mapper) of each gene; and the association of each CAG with experimental or clinical covariates is estimated (FIG. 9A). Considerations are made as to whether the CAGs associated with an outcome have member genes commonly represented by specific taxa. The identified taxa are used to identify operons associated with the outcome of interest, by aligning the member genes of associated CAGs to the reference genomes for that taxon.

Geneshot is used to analyze datasets from microbiome studies investigating the stool microbiome of subjects. The goal is to identify microbial gene groups (CAGs) that differed in relative abundance in a particular group as compared to another group, while allowing for differences in the baseline relative abundance of each CAG in the two cohorts.

To provide genomic and functional context for these CAGs, the dataset is next aligned to reference genomes from the taxonomic families.

In contrast to other end-to-end pipelines, the unit of analysis that underlies geneshot is de novo assembled protein-coding gene sequences, which are dimension-reduced via co-abundance clustering. One advantage of this gene-level approach to metagenomic analysis is a reduced reliance on reference databases (which are often incomplete) or inaccurate ontological hierarchies (e.g. the incompatibility of taxonomy with homoplasmy or horizontally transferred genetic elements). Moreover, by implementing an improved method for CAG construction geneshot is able to analyze gene-level abundances without being restricted to organismal groupings (such as MAGs or “metagenomic species”).

Geneshot's integration of reference and taxonomic databases derivies maximum benefit from the sparsely sequenced and annotated uncultivated organisms within the gut. After identifying CAGs associated with an outcome, it was then considered where the member genes of the associated CAGs have been observed in reference genomes. The actual organism representing these genes in a given specimen need not have a reference genome, just a somewhat related genomic island. Thus, geneshot maximizes what can be inferred from reference databases without being dependent upon the same references for identification of relevant genomic islands.

Finally, by wrapping together the set of tasks required for comprehensive gene-level metagenomics analysis using the Nextflow workflow management system a convenient mechanism is provided for microbiome analysis which can be implemented across a variety of high-performance computing infrastructures with minimal configuration required for each user. Geneshot can be used widely to enhance the quality of microbiome research, while also providing insights which may contribute to the development of microbiome-based therapeutics for cancer and other human diseases.

### Methods

### I. Bioinformatic Approach of Geneshot for Gene-Level Metagenomics

The process of performing gene-level metagenomic analysis is implemented by geneshot using a series of bioinformatic processes that are all coordinated into an overarching workflow using the free and open source Nextflow16 workflow management system. By providing options as “flags” to the workflow, users can modify the details of how the analysis is executed, and a record of the parameters used to execute the workflow can be saved (along with a summary of the computational resources which were used) with the reporting functionality provided by Nextflow. The complete set of code which constitutes geneshot can be found in the GitHub repository (github.com/Golob-Minot/geneshot), with documentation provided in the associated wiki.

The overview of the analytical tasks performed by geneshot are as follows:
1. Input WGS reads are preprocessed with adapter trimming using barcodecop and cutadapt and host reads are removed by subtractive alignment using BWA. The following steps use the FASTQ files which are output by this preprocessing step.
2. WGS reads from each specimen are de novo assembled individually using MegaHit and coding regions are predicted using Prodigal.
3. The conceptual translations of every predicted coding region are deduplicated across all specimens using a combination of linclust and DIAMOND, each of which applies a fixed threshold of amino acid similarity (default: 90%) and alignment coverage (default: 50%) to retain only the centroids from each cluster of coding sequences.
4. The preprocessed WGS reads from step 1 are then aligned against the gene catalog from step 3 in order to estimate the relative abundance of the organisms in each specimen which encode each gene. The alignment is carried out with DIAMOND (in blastx mode). Those alignments are then processed by FAMLI in order to resolve any reads which align equally well to multiple references by picking a single reference using an expectation maximization approach that uses a target metric of evenness of sequencing coverage across each reference as the target metric.
5. The relative abundance of each gene from the catalog across each specimen is computed as the depth of sequencing across each gene (the number of bases from WGS reads aligned to each gene divided by the length of the gene) divided by the sum of sequencing depth across all genes for that specimen. This provides a relative abundance estimate for the genomes encoding each gene in the source specimen that is adjusted for gene length.
6. Using the relative abundance of each gene across each specimen, genes are clustered using average linkage clustering and the cosine distance metric, applying a fixed distance metric to join genes into Co-Abundant Gene Groups (CAGs). Each gene from the catalog which is detected in at least one sample belongs to one and only one CAG. Two complementary approaches are used to provide a computationally tractable approach to this process: (a) genes are clustered in sub-groups (shards) which are iteratively combined over five sequential rounds; and (b) an Approximate Nearest Neighbor algorithm is used to rapidly index and retrieve co-abundant gene ‘neighborhoods’ to limit the search space for expensive computation of pairwise distance matrices as described previously. To mitigate any risk of the use of shards in (a) which could find sub-optimal clusters in early rounds, the stringency of the cosine distance threshold used to identify clusters is increased by a factor of two in all pre-clustering steps. Only in the final round of clustering is the user-specified cosine distance threshold applied, which results in the formation of CAGs which are the superset of the stringent groups formed in earlier rounds.
7. Independently from steps 4-6, the gene catalog may be optionally annotated by taxonomic classification using DIAMOND (which assigns the lowest common ancestor of top hits in blastp mode), as well as functional annotation using eggNOG-mapper. These annotation steps are optional and are executed if the user provides the reference databases required by either tool.
8. If the user provides a formula for statistical analysis using the variables defined in the manifest (the input file which is also used to label each pair of WGS FASTQ files with the appropriate specimen name), then the number of reads mapped to each CAG will be modeled using a beta-binomial model with that formula describing the logit of the expected relative abundance of the CAG. The model is fit using corncob. If taxonomic annotation was performed in step 7, taxon abundance coefficients are determined by aggregating the corncob results over all CAGs for which any constituent gene is taxonomically annotated as that taxon. The taxon-level estimated coefficients are then modelled using the errors-in-response model betta with no additional covariates (i.e., an intercept-only model is fit), and the hypothesis that the intercept is zero is tested. This approach allows an overall assessment of the effect of the covariate on the abundance of each taxon in a CAG-based analysis. Similarly, if functional annotation was performed in step 7, the same approach using betta and corncob is applied to all CAGs sharing the same functional annotation, which allows an overall assessment of the effect of the covariate on the abundance of each function. Taxon abundance coefficients are used in this analysis to prioritize reference genomes for more detailed analysis by exhaustive alignment of the genes from each CAG against individual reference genomes.
9. In the final step all of the outputs from each step are aggregated into HDF5 format, which organizes multiple tables into a single file using an internal organization structure to identify each of the elements of the results.

### II. Preparing Inputs for Geneshot

Prior to running geneshot, the following inputs are prepared: (1) a manifest in CSV format with columns for “specimen”, “R1”, “R2”, and any other additional metadata for statistical analysis, and (2) a set of FASTQ files (gzip-compressed) listed in the manifest, with forward and reverse reads listed separately in the “R1” and “R2” columns, respectively. In this format, a single specimen may be made up of multiple FASTQ file pairs by listing those files across multiple rows.

### III. Setting up Nextflow

The workflow management tool Nextflow is installed and configured. The complete documentation for Nextflow is publically available. The process of configuring Nextflow is accomplished by saving a “nextflow.config” file in the user's home directory, which describes the computing resources that should be used (local execution, SLURM, PBS, AWS, etc.), and which will be referenced when running geneshot.

### IV. Running Geneshot

Any of the the flags shown in Table 2 may be appended (e.g. --manifest manifest_file.csv).

TABLE 2
Description of flags used to customize parameters for running geneshot.

| Flag | Description |
|---|---|
| --manifest | CSV file listing samples (required) |
| --output | Folder to place analysis outputs (default: ./results) |
| --output_prefix | Text used as a prefix for summary HDF5 output files |
| | (default: geneshot) |
| --nopreprocess | If specified, omit the preprocessing steps (removing adapters |
| | and human sequences) |
| --savereads | If specified, save the preprocessed reads to the output folder |
| | (inside qc/) |
| --hg_index_url | URL for human genome index, defaults to current human |
| | genome |
| --hg_index | Cached copy of the bwa indexed human genome in .tar.gz |
| | format |
| --min_hg_align_score | Minimum alignment score for human genome (default 30) |
| --gene_fasta | (optional) Compressed FASTA with pre-generated catalog of |
| | microbial genes. If provided, then the entire de novo assembly |
| | process will be skipped entirely. |
| --min_identity | Amino acid percent identity cutoff used to combine similar |
| | genes (default: 90) |
| --min_coverage | Length percentage cutoff used to combine similar genes |
| | (default: 50) |
| --noannot | If specified, disable annotation for taxonomy or function. |
| --taxonomic_dmnd | Database used for taxonomic annotation (default: false) |
| --ncbi_taxdump | Reference describing the NCBI Taxonomy (default: |
| | ftp://ftp.ncbi.nlm.nih.gov/pub/taxonomy/taxdump.tar.gz) |
| --eggnog_dmnd | One of two databases used for functional annotation with |
| | eggNOG |
| | (default: false) |
| --eggnog_db | One of two databases used for functional annotation with |
| | eggNOG |
| | (default: false) |
| --dmnd_min_identity | Amino acid identity cutoff used to align short reads |
| | (default: 90) |
| --dmnd_min_coverage | Query coverage cutoff used to align short reads (default: 50) |
| --dmnd_top_pct | Keep top X % of alignments for each short read (default: 1) |
| --dmnd_min_score | Minimum score for short read alignment (default: 20) |
| --gencode | Genetic code used for conceptual translation (default: 11) |
| --sd_mean_cutoff | Ratio of standard deviation/mean depth of sequencing used |
| | to filter genes with FAMLI (default: 3.0) |
| --famli_batchsize | Number of alignments to deduplicate in batches (default: |
| | 10000000) |
| --distance_metric | Distance metric used to group genes by co-abundance |
| | (default: cosine) |
| --distance_threshold | Distance threshold used to group genes by co-abundance |
| | (default: 0.25) |
| --linkage_type | Linkage type used to group genes by co-abundance (default: |
| | average) |
| --formula | Optional formula used to estimate associations with CAG |
| | relative abundance |
| --fdr_method | FDR method used to calculate q-values for associations |
| | (default: ‘fdr_bh’) |
| --corncob_batches | Number of parallel processes to use processing each formula |
| | --composition |
| --composition | When included, metaPhlAn2 will be run on all specimens |

### V. Reference Databases

Annotation of the gene catalog generated by geneshot is performed using reference databases for DIAMOND and eggNOG-mapper. The following databases used for this analysis are generated based on the documentation in the associated tools:
Taxonomic annotation is based on a DIAMOND index of NCBI's RefSeq genome database generated on Jan. 15, 2020
Functional annotation is based on eggNOG-mapper v5.0 database downloaded on Jul. 17, 2020

### VI. Bioinformatic Component Tools and Dependencies

The images used by geneshot are listed in Table 3.

TABLE 3
Description of software dependencies and versions used by geneshot.

| Tool | Reference | Docker image | Dockerfile source |
|---|---|---|---|
| barcodecop | https://github.com/nhoffman/ | quay.io/fhcrc-microbiome/ | https://github.com/Gol ob- |
| | barcodecop | barcodecop:barcodecop_0.5.3 | Minot/docker/tree/ |
| | | | ma ster/barcodecop |
| cutadapt | MARTIN, Marcel. Cutadapt | quay.io/fhcrc-microbiome/ | https://github.com/Gol ob- |
| | removes adapter sequences | cutadapt:cutadapt_2.3_bcw_0.3.1 | Minot/docker/tree/ |
| | from high-throughput | | ma ster/cutadapt |
| | sequencing reads. | | |
| | EMBnet.journal, [S.l.], v. | | |
| | 17, n. 1, p. pp. 10-12, May 2011. | | |
| | ISSN 2226-6089. | | |
| BWA | Li H. and Durbin R. (2009) | quay.io/fhcrc- | https://github.com/ |
| | Fast and accurate short read | microbiome/bwa:bwa. | Fre dHutch/docker-bwa |
| | alignment with Burrows- | 0.7.17_bcw.0.3.0 | |
| | Wheeler Transform. | I | |
| | Bioinformatics, 25: 1754-60. | | |
| | [PMID: 19451168] | | |
| MegaHit | Li D, Liu C M, Luo R, | quay.io/biocontainers/ | https://github.com/ |
| | Sadakane K, Lam T W. | megahit:1.2.9--h8b12597_0 | vou tcn/megahit |
| | MEGAHIT: an | | |
| | ultra-fast single-node | | |
| | solution for large and | | |
| | complex metagenomics | | |
| | assembly via succinct de | | |
| | Bruijn graph. | | |
| | Bioinformatics. | | |
| | 2015; 31(10): 1674-1676. | | |
| | doi: 10.1093/bioinformatics/ | | |
| | btv033 | | |
| Prodigal | Hyatt D, Chen G L, | quay.io/biocontainers/ | https://github.com/Bio |
| | Locascio P F, Land M L, | prodigal:2.6.3--h516909a_2 | Containers/containers/ |
| | Larimer F W, Hauser L J. | | tree/master/prodigal |
| | Prodigal: prokaryotic gene | | |
| | recognition and translation | | |
| | initiation site identification. | | |
| | BMC Bioinformatics. | | |
| | 2010; 11: 119. | | |
| | Published 2010, Mar. 8. | | |
| | doi: 10.1186/1471-2105-11-119 | | |
| DIAMOND | Buchfink B, Xie C, Huson | quay.io/fhcrc- | https://github.com/ |
| | D H. Fast and sensitive | microbiome/famli:v1.5 | Fre dHutch/famli |
| | protein alignment using | | |
| | DIAMOND. Nat Methods. | | |
| | 2015; 12(1): 59-60. | | |
| | doi: 10.1038/nmeth.3176 | | |
| linclust | Steinegger M, Söding J. | quay.io/fhcrc- | https://github.com/ |
| | Clustering huge protein | microbiome/integrate- | Fre dHutch/integrate- |
| | sequence sets in linear time. | metagenomic-assemblies:v0.5 | metagenomic-assemblies |
| | Nat Commun. | | |
| | 2018; 9(1): 2542. Published | | |
| | 2018, Jun. 29. | | |
| | doi: 10.1038/s41467-018-04964-5 | | |
| FAMLI | In Silico Benchmarking of | quay.io/fhcrc- | https://github.com/ |
| | Metagenomic Tools for | microbiome/famli:v1.5 | Fre dHutch/famli |
| | Coding Sequence Detection | | |
| | Reveals the Limits of | | |
| | Sensitivity and Precision | | |
| | Jonathan Louis Golob, | | |
| | Samuel Schwartz Minot | | |
| | BMC Bioinformatics, | | |
| | accepted. | | |
| | doi: https://doi.org/10.1101/295352 | | |
| ANN- | Minot, S. S., Willis, A. D. | quay.io/fhcrc- | https://github.com/ |
| indexed | Clustering co-abundant | microbiome/find- | Fre dHutch/find-cags |
| gene | genes identifies components | cags:v0.13.0 | |
| clustering | of the gut microbiome that | | |
| | are reproducibly associated | | |
| | with colorectal cancer and | | |
| | inflammatory bowel | | |
| | disease. Microbiome 7, 110 | | |
| | (2019). | | |
| eggNOG- | Huerta-Cepas J, Forslund K, | quay.io/biocontainers/eggnog- | https://github.com/ |
| mapper | Coelho LP, et al. Fast | mapper:2.0.1-- | jhc epas/eggnog- |
| | Genome-Wide Functional | py_1 | mapper |
| | Annotation through | | |
| | Orthology Assignment by | | |
| | eggNOG-Mapper. Mol Biol | | |
| | Evol. 2017; 34(8): 2115- | | |
| | 2122. | | |
| | doi: 10.1093/molbev/msx148 | | |
| HDF5 | N/A | quay.io/fhcrc- | https://github.com/ |
| formatting | | microbiome/python- | fredhutch/docker- |
| (python-pandas) | | pandas:v1.0.3 | python- pandas |

### VII. Genome Alignments

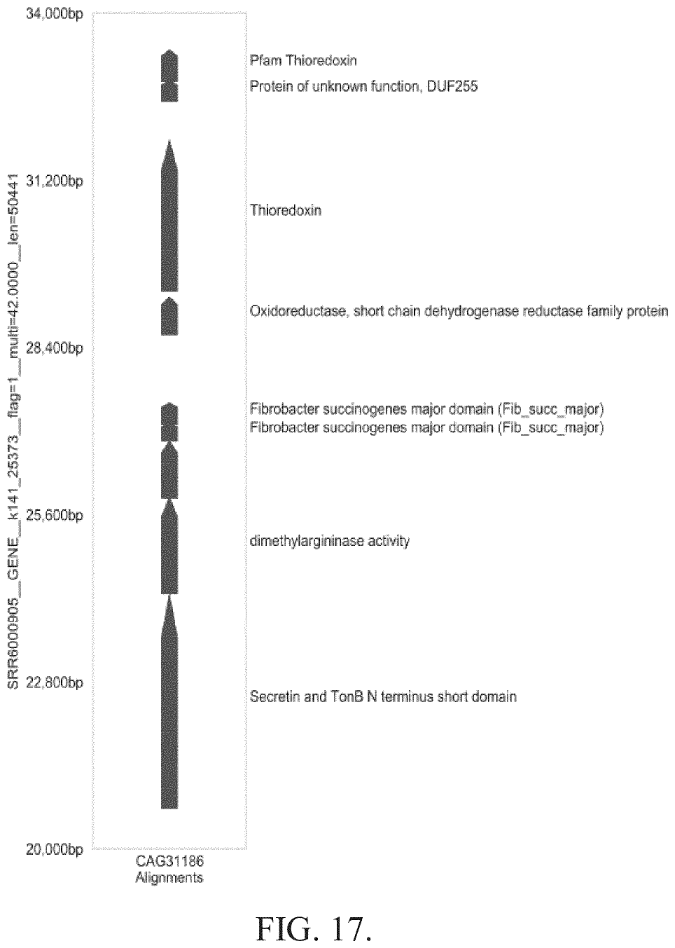

The process of aligning genes from the geneshot output against a set of reference genomes is executed the Annotation of Microbial Genomes by Microbiome Association (AMGMA) tool. This tool aligns genes from the geneshot gene catalog against a user-specified set of microbial genomes and then calculates summary metrics for each genome including the proportion of the genome which is aligned by genes which are associated with a given parameter. The genome alignment figures show the position of the genome alignment for the set of genes contained in the CAG of interest, as well as the location and annotation of genes recorded in GFF format within the NCBI RefSeq database. Alignment is performed using DIAMOND (using the dependencies shown in Table 3) with the complete set of Python code used to aggregate alignment results available at https://github.com/FredHutch/AMGMA/. An example of alignment to de novo assembled contigs, performed with the same set of steps, is shown in FIG. 17.

### VIII. Datasets Used for Analysis

The data analyzed includes the publicly available FASTQ records and associated metadata available.

### Example 5

### Geneshot for Identification of Genome Islands Associated with Immunotherapy Response in Subjects with Metastatic Melanoma

Microbial genomic islands associated with response to immune checkpoint inhibitor (ICI)-based cancer treatment were identified using geneshot, a bioinformatics tool for identifying testable hypotheses based on gene-level metagenomic analysis of WGS microbiome data. The bioinformatics tool described here is used for identifying testable hypotheses based on gene-level metagenomic analysis of WGS microbiome data (geneshot).

By applying geneshot to two independent previously published cohorts, microbial genomic islands consistently associated with response to immune checkpoint inhibitor (ICI)-based cancer treatment in culturable type strains were identified. The identified genomic islands are within operons involved in type II secretion, TonB-dependent transport, and bacteriophage growth. These results, as well as the underlying methodology, inform further mechanistic studies and facilitate the development of microbiome-enhanced therapeutics.

Protein-coding microbial genes were identified and combined into CAGs and input into a computational analysis pipeline for gene-level metagenomic analysis called geneshot. Geneshot is an end-to-end analysis workflow for microbiome experiments using CAGs as the fundamental unit of analysis. In brief, WGS data from each specimen was preprocessed to remove human reads and assemble de novo; predicted protein-coding genes were then deduplicated to make a reference gene catalog; the abundance of each gene in each specimen was estimated by assignment of raw reads against that gene catalog after rigorous handling of multiply-mapping reads; genes were grouped into CAGs based on their co-abundance observed across the samples; the possible taxonomic origins were identified (by alignment against RefSeq) and functionally (using eggNOG mapper) of each gene; and the association of each CAG with experimental or clinical covariates was estimated (FIG. 9A). Considerations were made as to whether the CAGs associated with an outcome have member genes commonly represented by specific taxa. The identified taxa were used to identify operons associated with the outcome of interest, by aligning the member genes of associated CAGs to the reference genomes for that taxon.

Geneshot was employed to reanalyze data from metagenomic studies in people who have received immune checkpoint inhibitors (ICI) for cancer. ICI are a class of immunotherapies that can induce robust and long-lasting protection from cancer. However, across cancer indications, the majority of subjects have no objective response to ICI treatment. Identifying the mechanisms that regulate response to ICI is an area of intense research, and multiple studies have demonstrated that the gut microbiome can regulate the anti-tumor response induced by ICI in distant tissues, such as the lung and skin. Further, transfer of the gut microbiome from subjects to gnotobiotic mice transfers ICI responsiveness to the new host. Taxonomic analysis of these datasets to identify the microbes responsible for transferring ICI responsiveness has yielded inconsistent results across cohorts. It is possible that the microbes driving the ICI-response phenotype may truly vary across these studies (e.g., due to biogeographical variation in human microbiome composition or the methodologies used to classify responder status), but the identification of common microbial genomic features associated with ICI response across multiple cohorts using gene-level analysis will accelerate understanding the mechanistic basis of the transfer of ICI responsiveness by the microbiome.

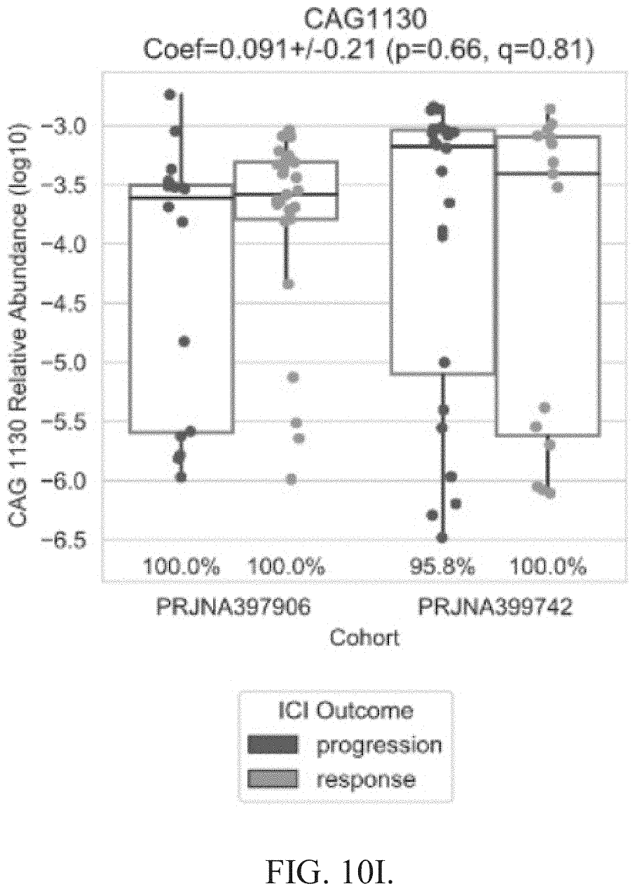

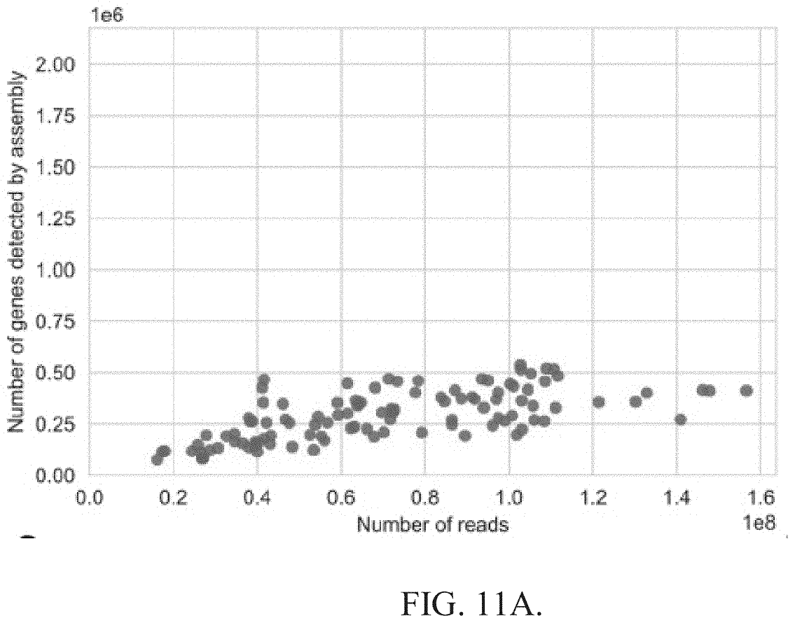

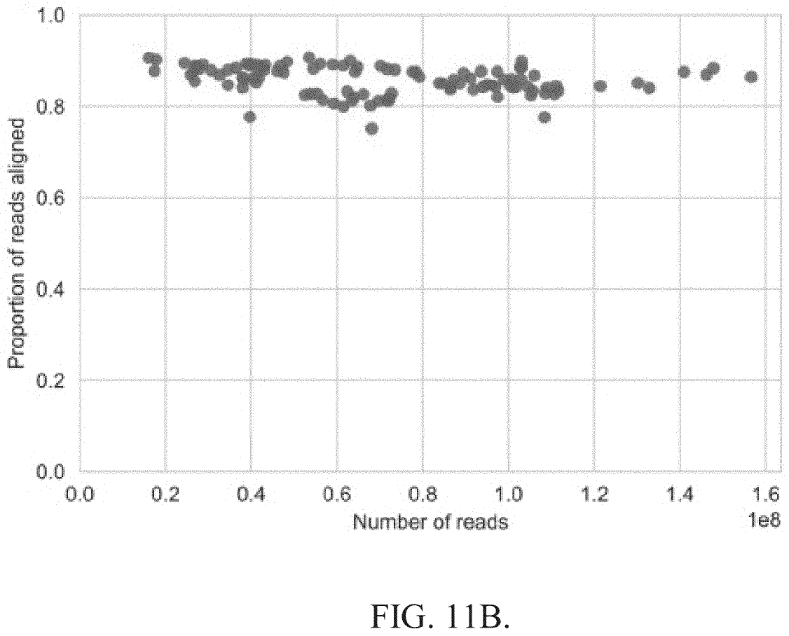

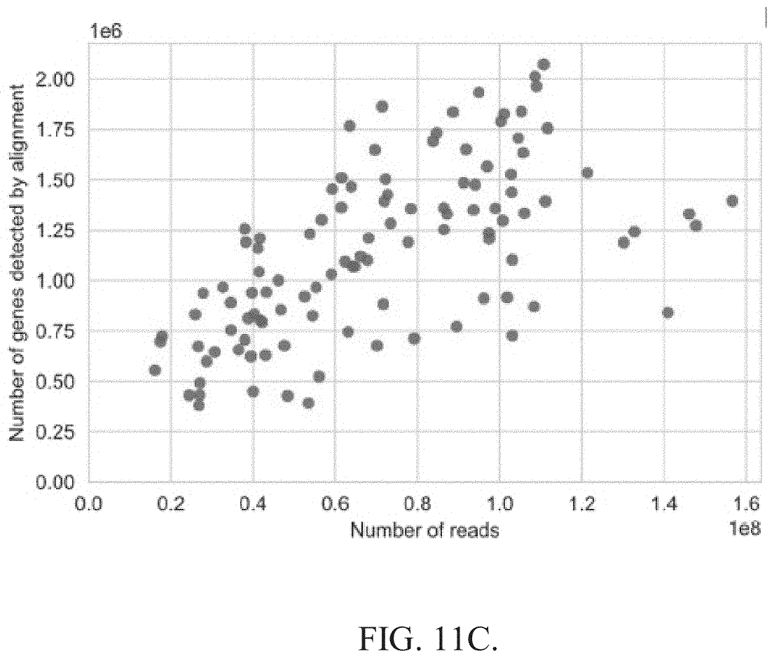

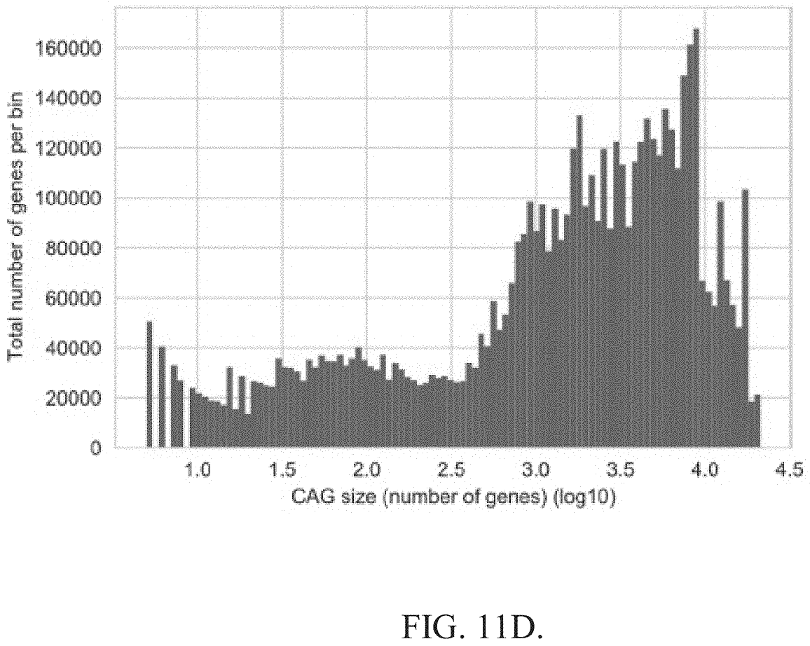

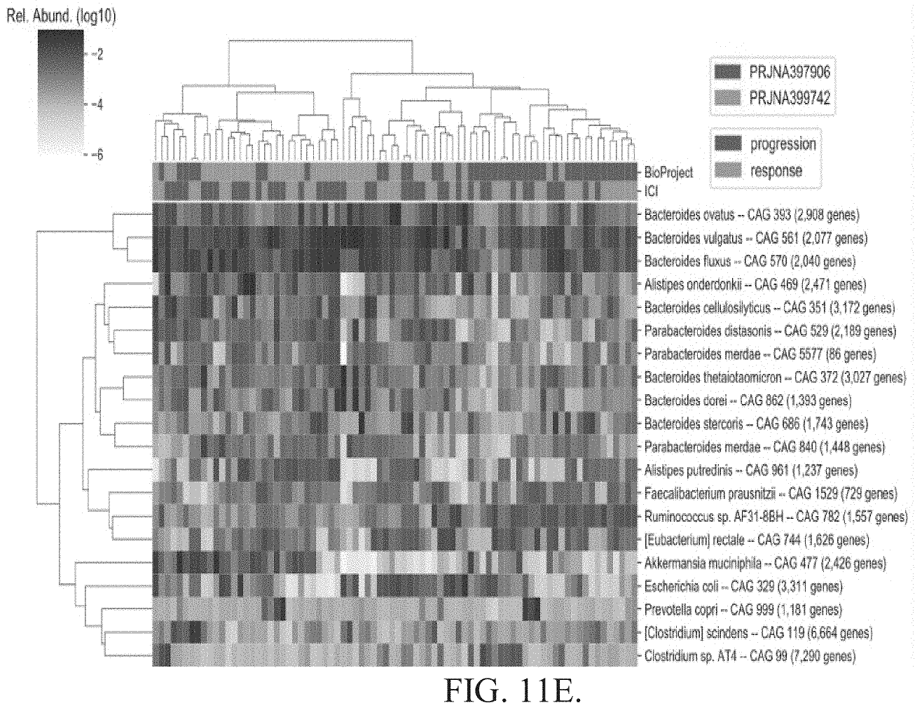

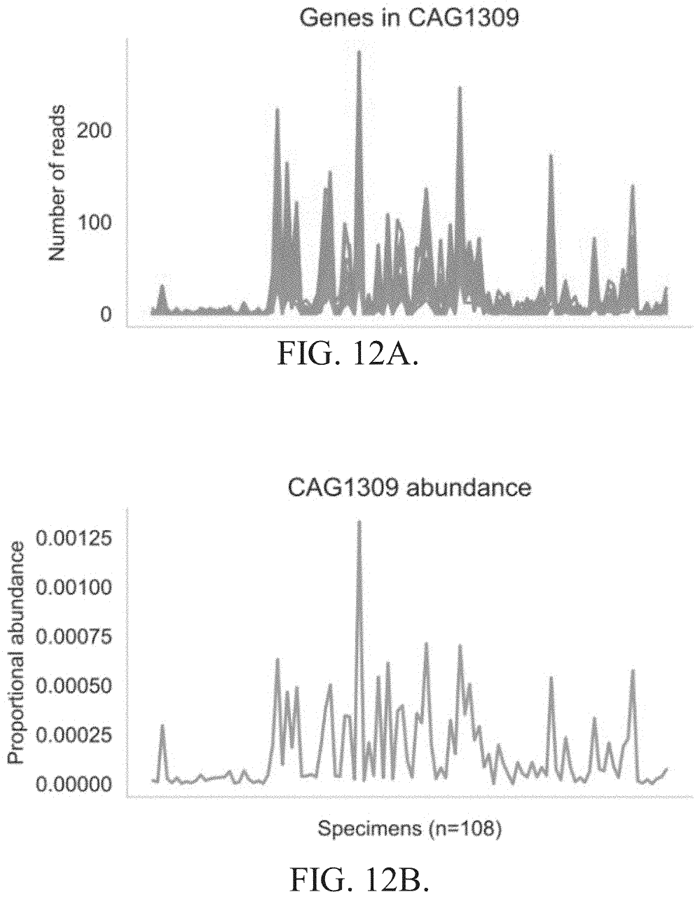

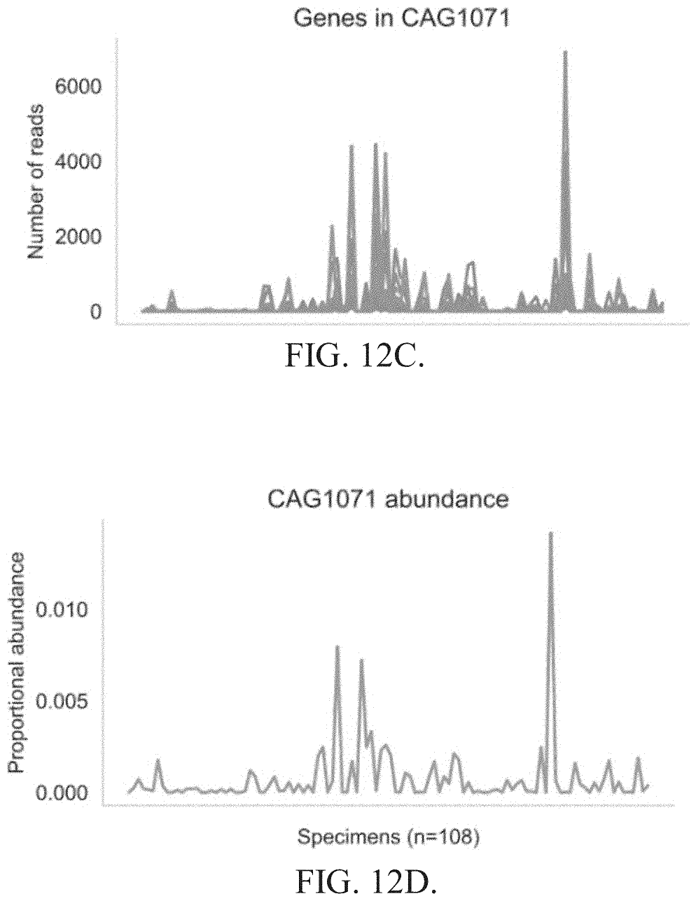

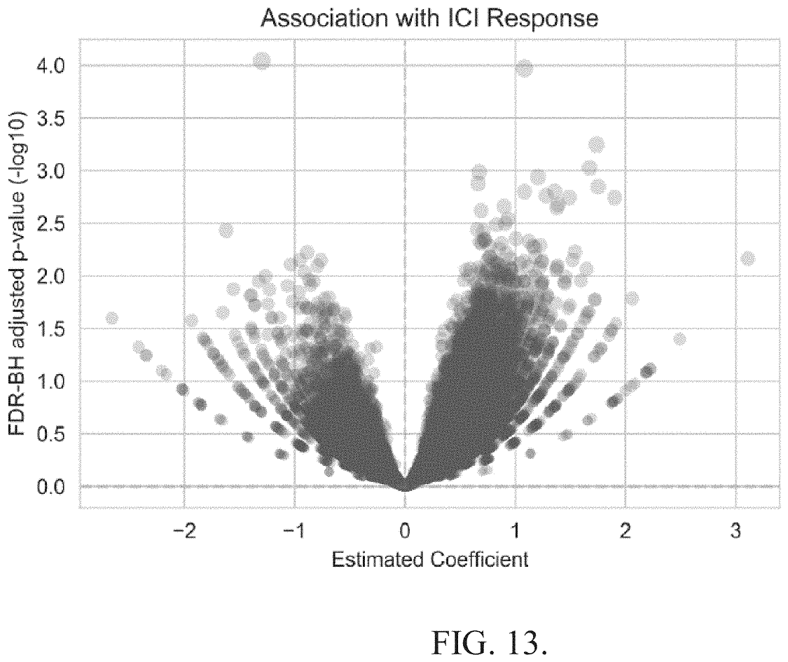

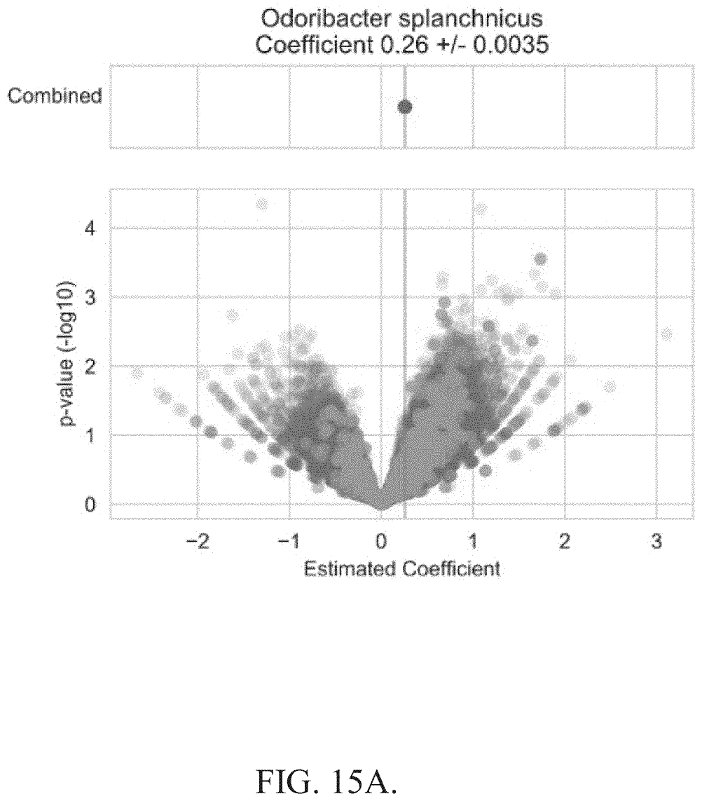

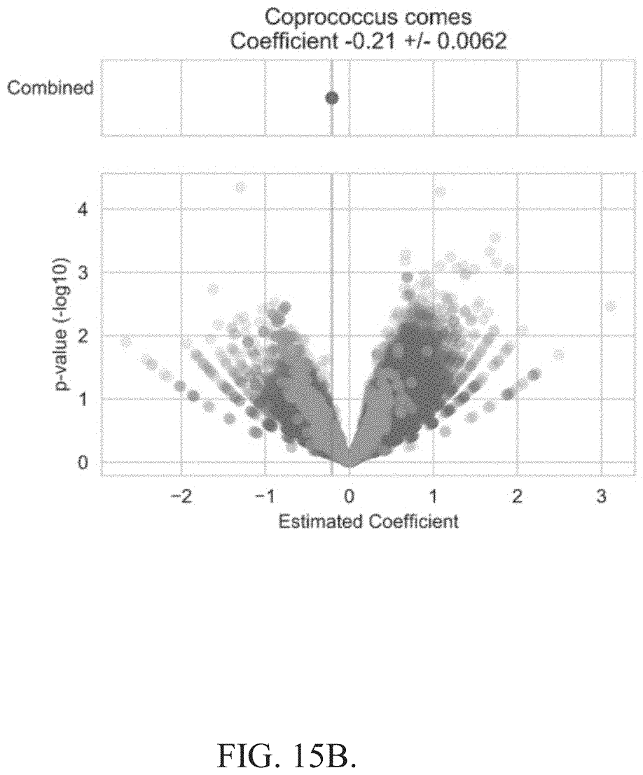

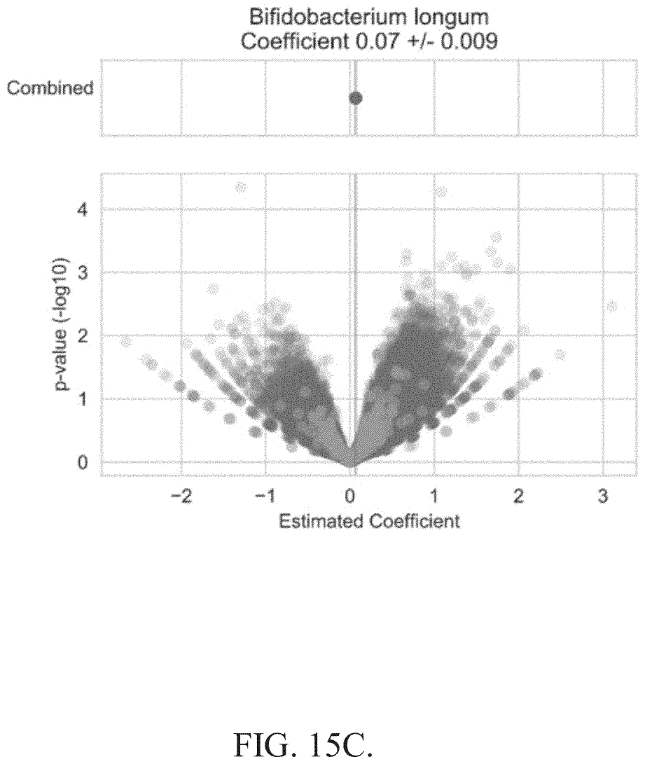

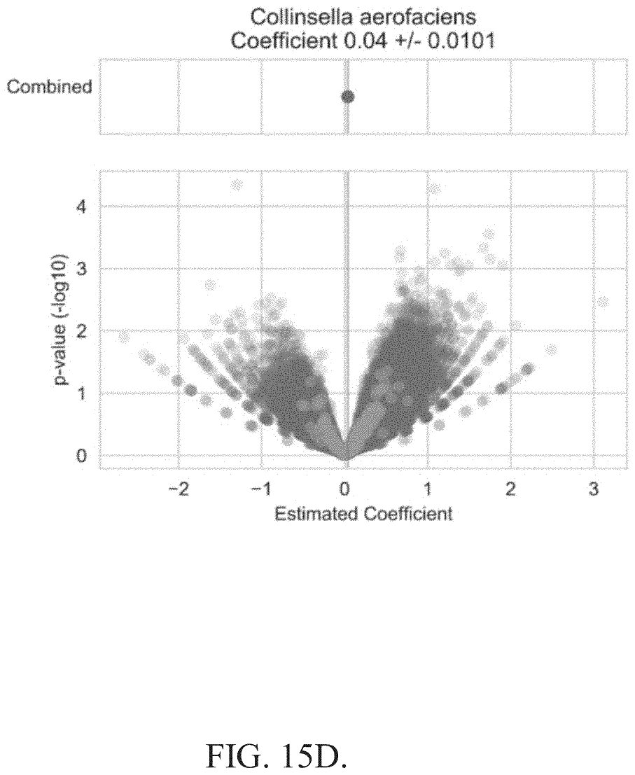

Geneshot was used to analyze published datasets from microbiome studies investigating the stool microbiome of participants receiving ICI therapy for metastatic melanoma treatment. The goal was to identify microbial gene groups (CAGs) that differed in relative abundance in ICI responders compared to non-responders (termed ‘progressors’), while allowing for differences in the baseline relative abundance of each CAG in the two cohorts. Each stool microbiome sample yielded 75,488-536,005 (median 280,065) microbial genes by de novo assembly which were deduplicated to form a gene catalog of 7,209,758 unique protein-coding sequences (FIG. 9B; FIG. 11A). The majority of raw WGS reads (median 86.1%) were uniquely assigned to that gene catalog by translated nucleotide alignment, and 380,202-2,071,835 (median 1,174,474) genes were detected in each sample (FIG. 11B-11C). Co-abundance clustering of the genes yielded 1,232,769 distinct CAGs; member genes within the most abundant CAGs were commonly represented in the reference genomes of common gut residents (FIG. 11D-11E; FIG. 12A-12D). With an FDR threshold of 0.01, 3.019 CAGs (4,509 genes in total) were found to be significantly associated with ICI response, with 2,634 CAGs associated with progression and 385 CAGs associated with response (FIG. 13). These associated CAGs had member genes commonly present in Odoribacter splanchnicus and Gemmiger formicilis (which were found to be more abundant in responders), as well as Coprococcus comes (which was more abundant in progressors) (FIGS. 14A-14E, FIGS. 15A-15D).

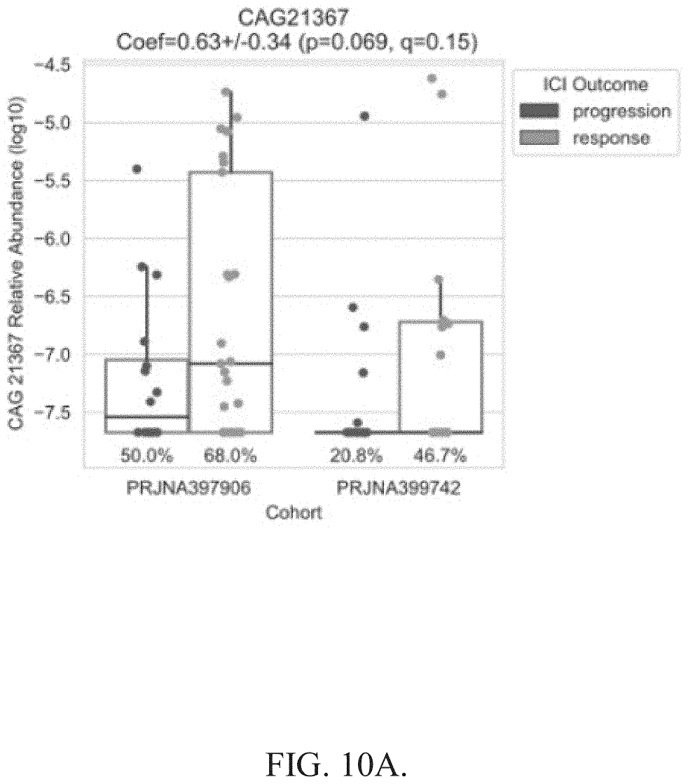

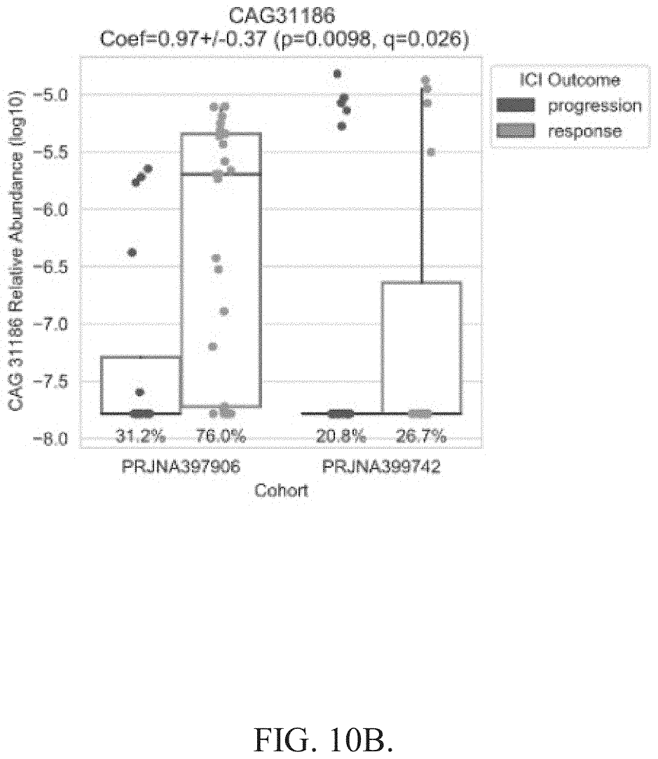

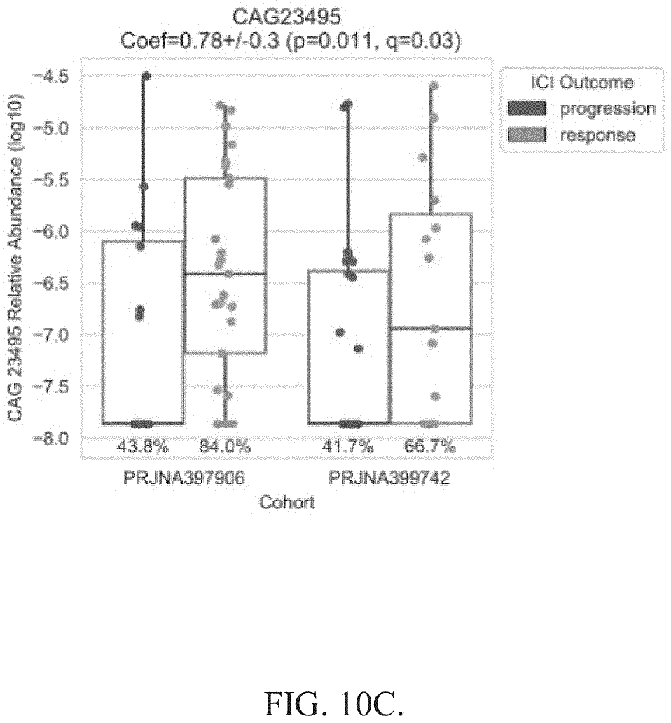

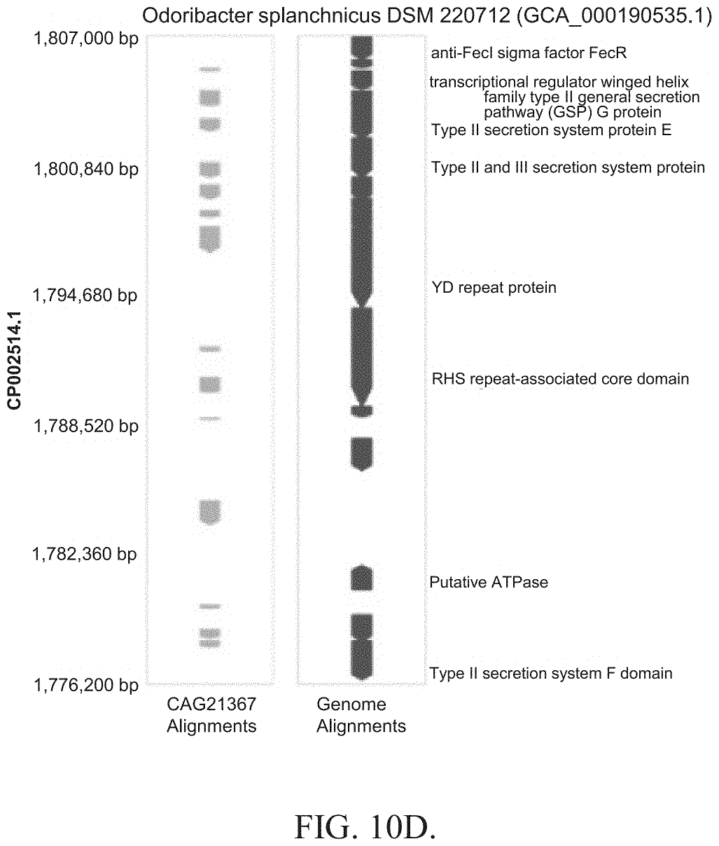

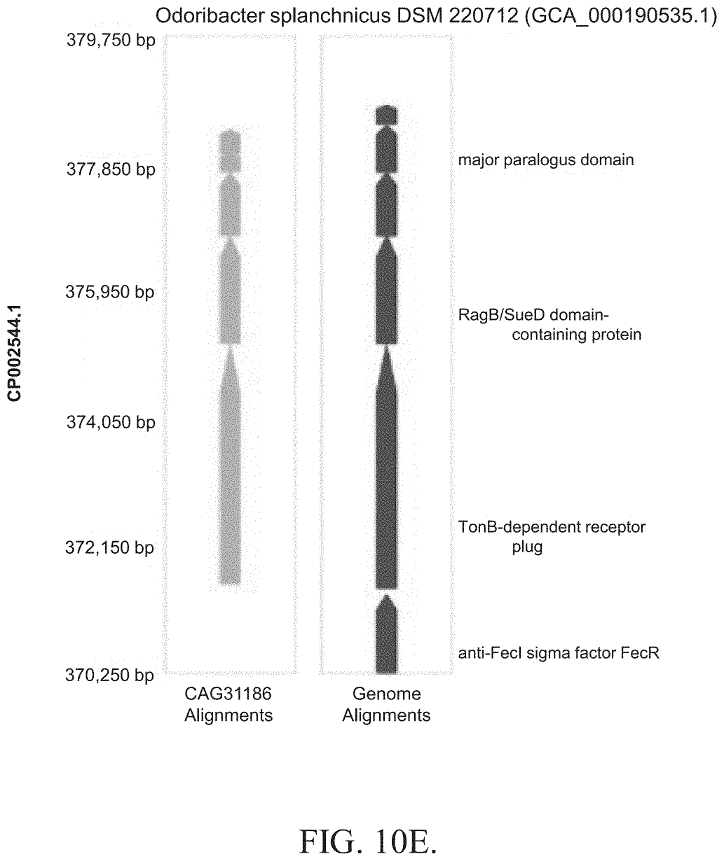

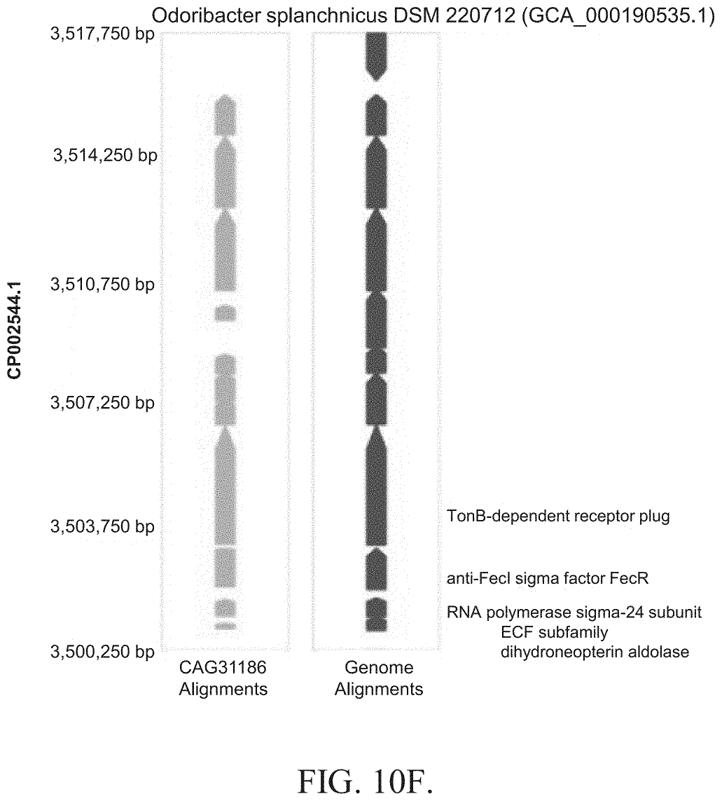

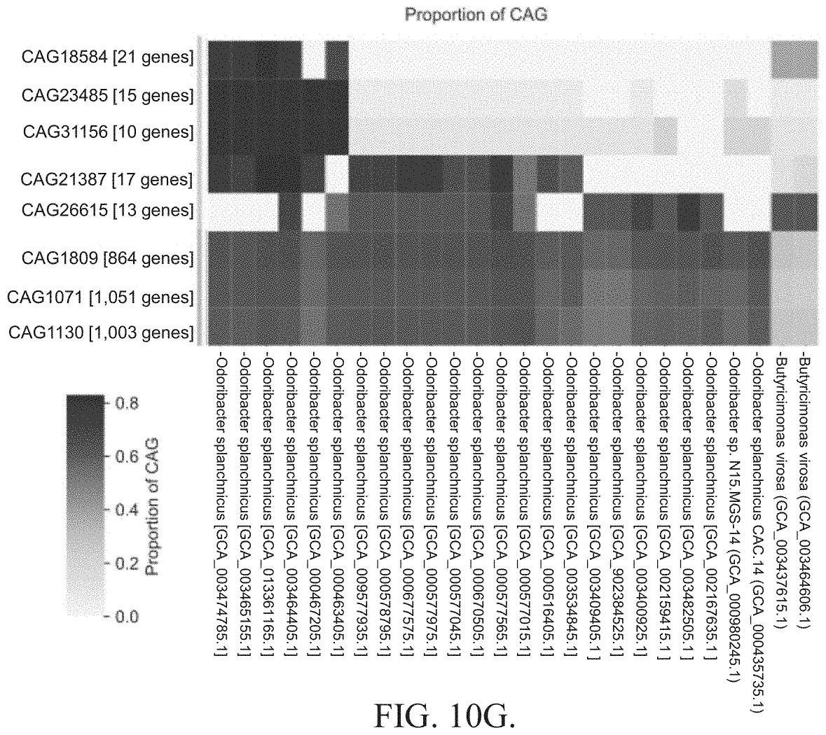

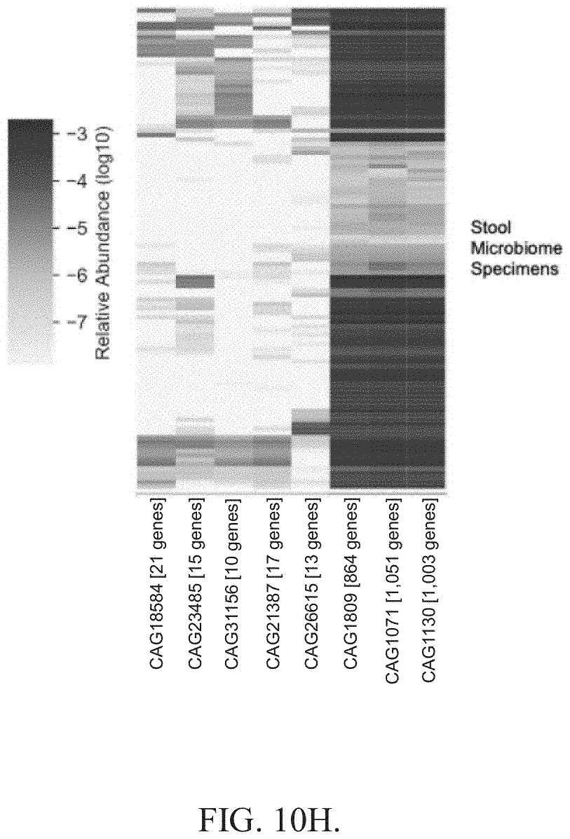

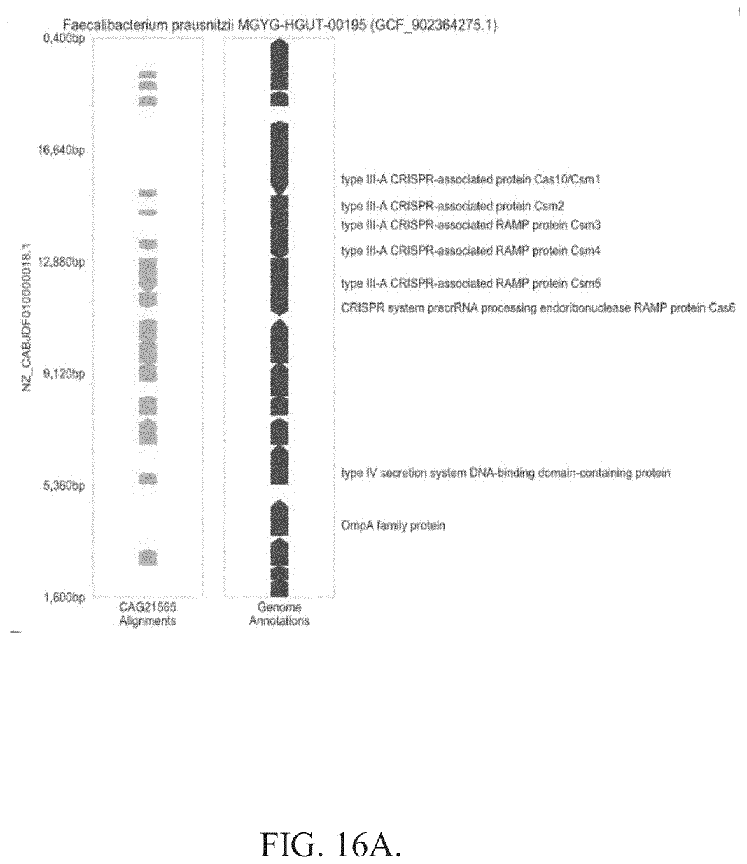

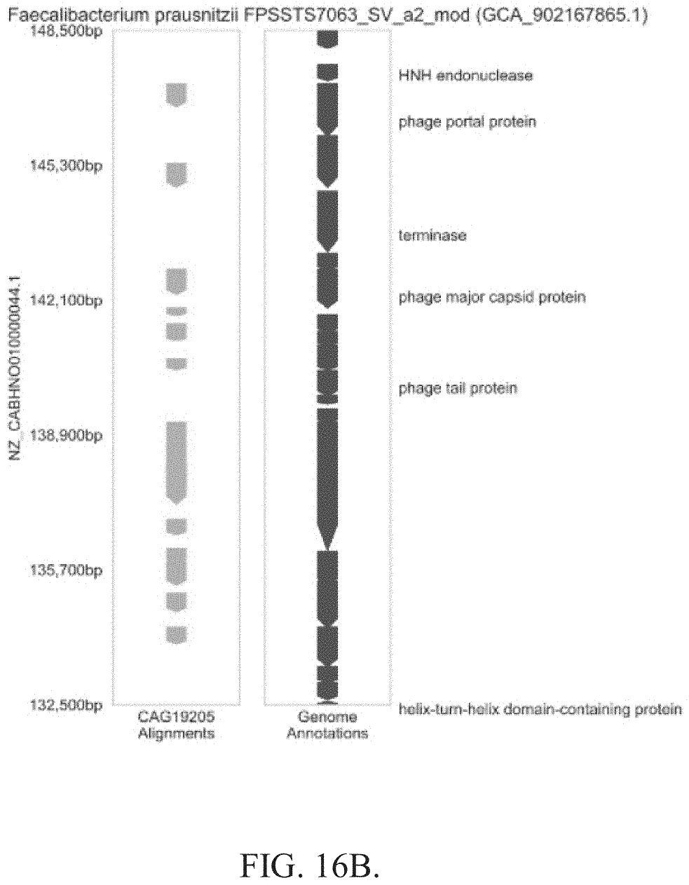

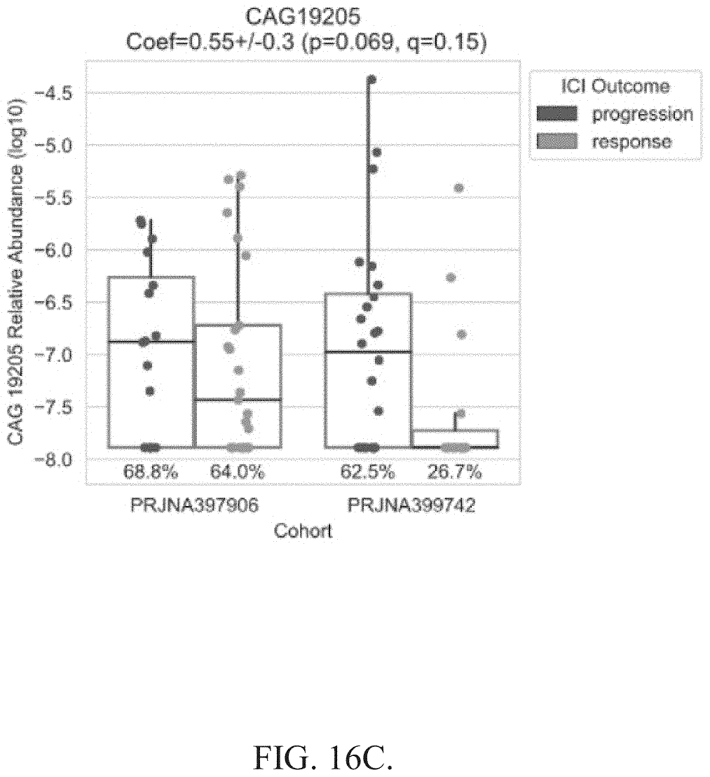

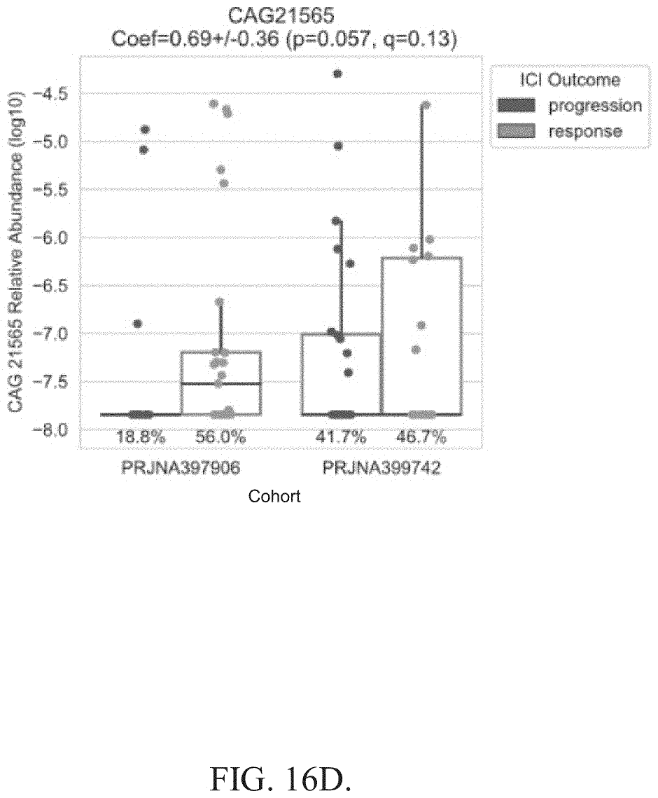

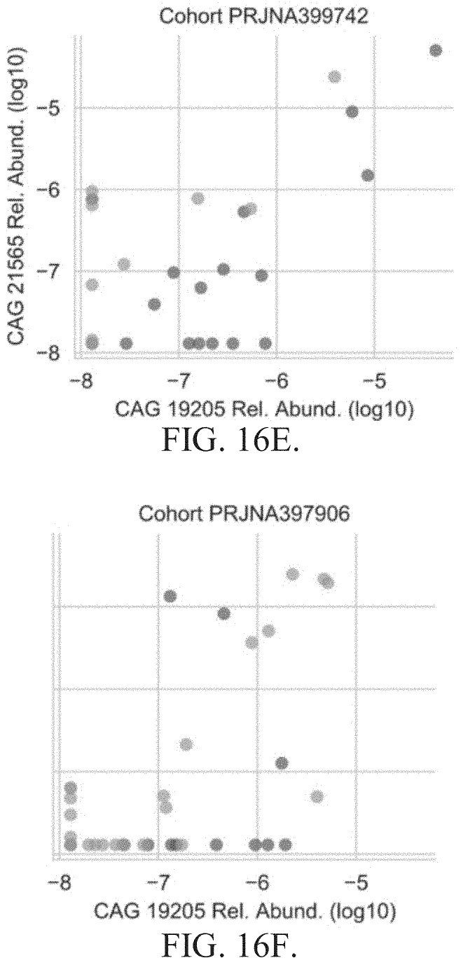

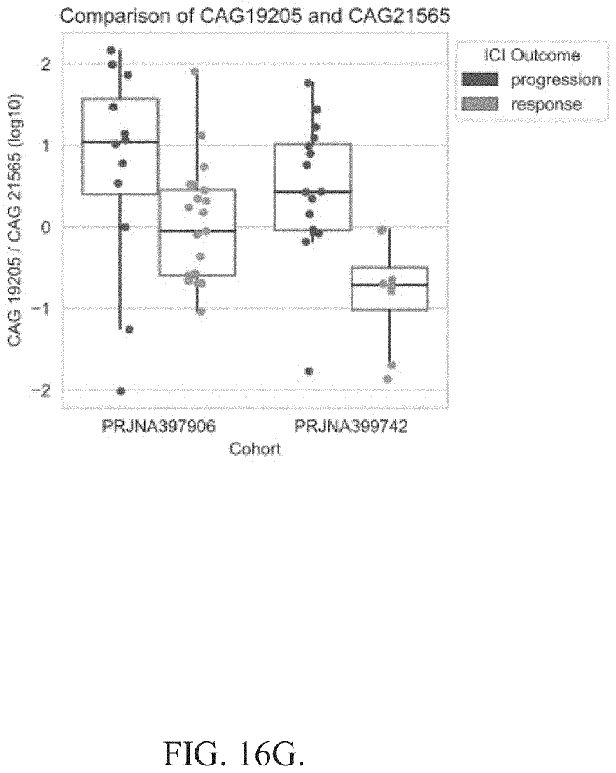

To provide genomic and functional context for these ICI-associated CAGs, the dataset was next aligned to 1,886 reference genomes from the taxonomic families shown in FIG. 14C. Rather than being spread throughout the genome, the genes comprising the CAGs were found in contiguous genomic regions (“islands”) ranging in size from 10-35 kb in size. ICI-associated CAGs aligning to Odoribacter splanchnicus DSM 220712 corresponded to strain-specific genomic islands encoding type II secretion and TonB-dependent transport, which were found at higher abundance in the gut of individuals who responded to ICI therapy across both cohorts (FIGS. 10A-10I). Genomic islands in Clostridium sp. HMb25 and Faecalibacterium prausnitzii were annotated as integrated prophages, with an additional genomic island in F. prausnitzii annotated with a CRISPR defense system, supporting a role of bacteriophage growth in the ICI microbiome interaction (FIGS. 16A-16G). Thus, geneshot was able to identify not only potentially relevant taxa, but also strain specific genomic islands. Furthermore, geneshot identified specific possible mechanisms (type II secretion, TonB-dependent transport, and phage) that are suitable for targeted hypothesis testing (e.g., in model systems).

In contrast to other end-to-end pipelines, the unit of analysis that underlies geneshot is de novo assembled protein-coding gene sequences, which are dimension-reduced via co-abundance clustering. One advantage of this gene-level approach to metagenomic analysis is a reduced reliance on reference databases (which are often incomplete) or inaccurate ontological hierarchies (e.g. the incompatibility of taxonomy with homoplasmy or horizontally transferred genetic elements). Moreover, by implementing an improved method for CAG construction geneshot is able to analyze gene-level abundances without being restricted to organismal groupings (such as MAGs or “metagenomic species”).

Geneshot's integration of reference and taxonomic databases derivies maximum benefit from the sparsely sequenced and annotated uncultivated organisms within the gut. After identifying CAGs associated with an outcome, it was then considered where the member genes of the associated CAGs have been observed in reference genomes. The actual organism representing these genes in a given specimen need not have a reference genome, just a somewhat related genomic island. Thus, geneshot maximizes what can be inferred from reference databases without being dependent upon the same references for identification of relevant genomic islands.

Finally, by wrapping together the set of tasks required for comprehensive gene-level metagenomics analysis using the Nextflow workflow management system a convenient mechanism is provided for microbiome analysis which can be implemented across a variety of high-performance computing infrastructures with minimal configuration required for each user. Geneshot can be used widely to enhance the quality of microbiome research, while also providing insights which may contribute to the development of microbiome-based therapeutics for cancer and other human diseases.

### Methods

### I. Bioinformatic Approach of Geneshot for Gene-Level Metagenomics

The process of performing gene-level metagenomic analysis was implemented by geneshot using a series of bioinformatic processes which are all coordinated into an overarching workflow using the free and open source Nextflow16 workflow management system. By providing options as “flags” to the workflow, users can modify the details of how the analysis is executed, and a record of the parameters used to execute the workflow can be saved (along with a summary of the computational resources which were used) with the reporting functionality provided by Nextflow. The complete set of code which constitutes geneshot can be found in the GitHub repository (github.com/Golob-Minot/geneshot), with documentation provided in the associated wiki.

The overview of the analytical tasks performed by geneshot are as follows:
1. Input WGS reads are preprocessed with adapter trimming using barcodecop and cutadapt and host reads are removed by subtractive alignment using BWA. The following steps use the FASTQ files which are output by this preprocessing step.
2. WGS reads from each specimen are de novo assembled individually using MegaHit and coding regions are predicted using Prodigal.
3. The conceptual translations of every predicted coding region are deduplicated across all specimens using a combination of linclust and DIAMOND, each of which applies a fixed threshold of amino acid similarity (default: 90%) and alignment coverage (default: 50%) to retain only the centroids from each cluster of coding sequences.
4. The preprocessed WGS reads from step 1 are then aligned against the gene catalog from step 3 in order to estimate the relative abundance of the organisms in each specimen which encode each gene. The alignment is carried out with DIAMOND (in blastx mode). Those alignments are then processed by FAMLI in order to resolve any reads which align equally well to multiple references by picking a single reference using an expectation maximization approach that uses a target metric of evenness of sequencing coverage across each reference as the target metric.
5. The relative abundance of each gene from the catalog across each specimen is computed as the depth of sequencing across each gene (the number of bases from WGS reads aligned to each gene divided by the length of the gene) divided by the sum of sequencing depth across all genes for that specimen. This provides a relative abundance estimate for the genomes encoding each gene in the source specimen that is adjusted for gene length.
6. Using the relative abundance of each gene across each specimen, genes are clustered using average linkage clustering and the cosine distance metric, applying a fixed distance metric to join genes into Co-Abundant Gene Groups (CAGs). Each gene from the catalog which is detected in at least one sample belongs to one and only one CAG. Two complementary approaches are used to provide a computationally tractable approach to this process: (a) genes are clustered in sub-groups (shards) which are iteratively combined over five sequential rounds; and (b) an Approximate Nearest Neighbor algorithm is used to rapidly index and retrieve co-abundant gene ‘neighborhoods’ to limit the search space for expensive computation of pairwise distance matrices as described previously. To mitigate any risk of the use of shards in (a) which could find sub-optimal clusters in early rounds, the stringency of the cosine distance threshold used to identify clusters is increased by a factor of two in all pre-clustering steps. Only in the final round of clustering is the user-specified cosine distance threshold applied, which results in the formation of CAGs which are the superset of the stringent groups formed in earlier rounds.
7. Independently from steps 4-6, the gene catalog may be optionally annotated by taxonomic classification using DIAMOND (which assigns the lowest common ancestor of top hits in blastp mode), as well as functional annotation using eggNOG-mapper. These annotation steps are optional and are executed if the user provides the reference databases required by either tool.
8. If the user provides a formula for statistical analysis using the variables defined in the manifest (the input file which is also used to label each pair of WGS FASTQ files with the appropriate specimen name), then the number of reads mapped to each CAG will be modeled using a beta-binomial model with that formula describing the logit of the expected relative abundance of the CAG. The model is fit using corncob. If taxonomic annotation was performed in step 7, taxon abundance coefficients are determined by aggregating the corncob results over all CAGs for which any constituent gene is taxonomically annotated as that taxon. The taxon-level estimated coefficients are then modelled using the errors-in-response model betta with no additional covariates (i.e., an intercept-only model is fit), and the hypothesis that the intercept is zero is tested. This approach allows an overall assessment of the effect of the covariate on the abundance of each taxon in a CAG-based analysis. Similarly, if functional annotation was performed in step 7, the same approach using betta and corncob is applied to all CAGs sharing the same functional annotation, which allows an overall assessment of the effect of the covariate on the abundance of each function. Taxon abundance coefficients are used in this analysis to prioritize reference genomes for more detailed analysis by exhaustive alignment of the genes from each CAG against individual reference genomes.
9. In the final step all of the outputs from each step are aggregated into HDF5 format, which organizes multiple tables into a single file using an internal organization structure to identify each of the elements of the results.

### II. Preparing Inputs for Geneshot

Prior to running geneshot, the following inputs were prepared: (1) a manifest in CSV format with columns for “specimen”, “R1”, “R2”, and any other additional metadata for statistical analysis, and (2) a set of FASTQ files (gzip-compressed) listed in the manifest, with forward and reverse reads listed separately in the “R1” and “R2” columns, respectively. In this format, a single specimen may be made up of multiple FASTQ file pairs by listing those files across multiple rows.

### III. Setting Up Nextflow

The workflow management tool Nextflow was installed and configured. The complete documentation for Nextflow is publically available. The process of configuring Nextflow was accomplished by saving a “nextflow.config” file in the user's home directory, which describes the computing resources that should be used (local execution, SLURM, PBS, AWS, etc.), and which will be referenced when running geneshot.

### IV. Running Geneshot

Any of the the flags shown in Table 2 may be appended (e.g. --manifest manifest_file.csv).

TABLE 2
Description of flags used to customize parameters for running geneshot.

| Flag | Description |
|---|---|
| --manifest | CSV file listing samples (required) |
| --output | Folder to place analysis outputs (default: ./results) |
| --output_prefix | Text used as a prefix for summary HDF5 output files |
| | (default: geneshot) |
| --nopreprocess | If specified, omit the preprocessing steps (removing adapters |
| | and human sequences) |
| --savereads | If specified, save the preprocessed reads to the output folder |
| | (inside qc/) |
| --hg_index_url | URL for human genome index, defaults to current human |
| | genome |
| --hg_index | Cached copy of the bwa indexed human genome in .tar.gz |
| | format |
| --min_hg_align_score | Minimum alignment score for human genome (default 30) |
| --gene_fasta | (optional) Compressed FASTA with pre-generated catalog of |
| | microbial genes. If provided, then the entire de novo assembly process |
| | will be skipped entirely. |
| --min_identity | Amino acid percent identity cutoff used to combine similar |
| | genes (default: 90) |
| --min_coverage | Length percentage cutoff used to combine similar genes |
| | (default: 50) |
| --noannot | If specified, disable annotation for taxonomy or function. |
| --taxonomic_dmnd | Database used for taxonomic annotation (default: false) |
| --ncbi_taxdump | Reference describing the NCBI Taxonomy |
| | (default: |
| | ftp://ftp.ncbi.nlm.nih.gov/pub/taxonomy/taxdump.tar.gz) |
| --eggnog_dmnd | One of two databases used for functional annotation with |
| | eggNOG |
| | (default: false) |
| --eggnog_db | One of two databases used for functional annotation with |
| | eggNOG |
| | (default: false) |
| --dmnd_min_identity | Amino acid identity cutoff used to align short reads (default: |
| | 90) |
| --dmnd_min_coverage | Query coverage cutoff used to align short reads (default: 50) |
| --dmnd_top_pct | Keep top X % of alignments for each short read (default: 1) |
| --dmnd_min_score | Minimum score for short read alignment (default: 20) |
| --gencode | Genetic code used for conceptual translation (default: 11) |
| --sd_mean_cutoff | Ratio of standard deviation/mean depth of sequencing used |
| | to filter genes with FAMLI (default: 3.0) |
| --famli_batchsize | Number of alignments to deduplicate in batches (default: |
| | 10000000) |
| --distance_metric | Distance metric used to group genes by co-abundance |
| | (default: cosine) |
| --distance_threshold | Distance threshold used to group genes by co-abundance |
| | (default: 0.25) |
| --linkage_type | Linkage type used to group genes by co-abundance (default: |
| | average) |
| --formula | Optional formula used to estimate associations with CAG |
| | relative abundance |
| --fdr_method | FDR method used to calculate q-values for associations |
| | (default: ‘fdr_bh’) |
| --corncob_batches | Number of parallel processes to use processing each formula |
| | --composition |
| --composition | When included, metaPhlAn2 will be run on all specimens |

### V. Reference Databases

Annotation of the gene catalog generated by geneshot was performed using reference databases for DIAMOND and eggNOG-mapper. The following databases used for this analysis were generated based on the documentation in the associated tools:
Taxonomic annotation is based on a DIAMOND index of NCBI's RefSeq genome database generated on Jan. 15, 2020
Functional annotation is based on eggNOG-mapper v5.0 database downloaded on Jul. 17, 2020

### VI. Bioinformatic Component Tools and Dependencies

The images used by geneshot are listed in Table 3.

TABLE 3
Description of software dependencies and versions used by geneshot.

| Tool | Reference | Docker image | Dockerfile source |
|---|---|---|---|
| barcodecop | https://github.com/ | quay.io/fhcrc-microbiome/ | https://github.com/ |
| | nhoffman/barcodecop | barcodecop:barcodecop_0.5.3 | Gol ob-Minot/docker/tree/ |
| | | | master/barcodecop |
| cutadapt | MARTIN, Marcel. Cutadapt | quay.io/fhcrc- | https://github.com/ |
| | removes adapter sequences | microbiome/cutadapt:cutadapt_2.3_bcw_0.3.1 | Gol ob-Minot/docker/tree/ |
| | from high-throughput | | ma ster/cutadapt |
| | sequencing reads. | | |
| | EMBnet.journal, [S.l.], v. 17, | | |
| | n. 1, p. pp. 10-12, May 2011. | | |
| | ISSN 2226-6089. | | |
| BWA | Li H. and Durbin R. (2009) | quay.io/fhcrc- | https://github.com/ |
| | Fast and accurate short read | microbiome/ | Fre dHutch/docker-bwa |
| | alignment with Burrows-Wheeler Transform. | bwa:bwa.0.7.17_bcw.0.3.0 I | |
| | Bioinformatics, 25: 1754-60. | | |
| | [PMID: 19451168] | | |
| MegaHit | Li D, Liu C M, Luo R, | quay.io/biocontainers/ | https://github.com/ |
| | Sadakane K, Lam T W. | megahit:1.2.9--h8b12597_0 | vou tcn/megahit |
| | MEGAHIT: an | | |
| | ultra-fast single-node | | |
| | solution for large and | | |
| | complex metagenomics | | |
| | assembly via succinct de | | |
| | Bruijn graph. | | |
| | Bioinformatics. | | |
| | 2015; 31(10): 1674-1676. | | |
| | doi: 10.1093/bioinformatics/ | | |
| | btv033 | | |
| Prodigal | Hyatt D, Chen G L, | quay.io/biocontainers/ | https://github.com/ |
| | Locascio P F, Land M L, | prodigal:2.6.3--h516909a_2 | Bio Containers/ |
| | Larimer F W, Hauser L J. | | containers/ |
| | Prodigal: prokaryotic gene | | tree/master/prodigal |
| | recognition and translation | | |
| | initiation site identification. | | |
| | BMC Bioinformatics. | | |
| | 2010; 11: 119. | | |
| | Published 2010, Mar. 8. | | |
| | doi: 10.1186/1471-2105-11-119 | | |
| DIAMOND | Buchfink B, Xie C, Huson | quay.io/fhcrc- | https://github.com/ |
| | D H. Fast and sensitive | microbiome/famli:v1.5 | FredHutch/famli |
| | protein alignment using | | |
| | DIAMOND. Nat Methods. | | |
| | 2015; 12(1): 59-60. | | |
| | doi: 10.1038/nmeth.3176 | | |
| linclust | Steinegger M, Söding J. | quay.io/fhcrc- | https://github.com/ |
| | Clustering huge protein | microbiome/ | FredHutch/integrate- |
| | sequence sets in linear time. | integrate- metagenomic- | metagenomic- |
| | Nat Commun. | assemblies:v0.5 | assemblies |
| | 2018; 9(1): 2542. Published | | |
| | 2018, Jun. 29. | | |
| | doi: 10.1038/s41467-018-04964-5 | | |
| FAMLI | In Silico Benchmarking of | quay.io/fhcrc- | https://github.com/ |
| | Metagenomic Tools for | microbiome/famli:v1.5 | Fre dHutch/famli |
| | Coding Sequence Detection | | |
| | Reveals the Limits of | | |
| | Sensitivity and Precision | | |
| | Jonathan Louis Golob, | | |
| | Samuel Schwartz Minot | | |
| | BMC Bioinformatics, | | |
| | accepted, doi: | | |
| | https://doi.org/10.1101/295352 | | |
| ANN- | Minot, S. S., Willis, A. D. | quay.io/fhcrc- | https://github.com/ |
| indexed | Clustering co-abundant | microbiome/find- | Fre dHutch/find- |
| gene | genes identifies components | cags:v0.13.0 | cags |
| clustering | of the gut microbiome that | | |
| | are reproducibly associated | | |
| | with colorectal cancer and | | |
| | inflammatory bowel | | |
| | disease. Microbiome 7, 110 | | |
| | (2019). | | |
| eggNOG- | Huerta-Cepas J, Forslund K, | quay.io/biocontainers/ | https://github.com/ |
| mapper | Coelho L P, et al. Fast | eggnog- | jhc epas/eggnog- |
| | Genome-Wide Functional | mapper:2.0.1-- | mapper |
| | Annotation through | py_1 | |
| | Orthology Assignment by | | |
| | eggNOG-Mapper. Mol Biol | | |
| | Evol. 2017; 34(8): 2115- | | |
| | 2122. | | |
| | doi: 10.1093/molbev/msx148 | | |
| HDF5 | N/A | quay.io/fhcrc- | https://github.com/ |
| formatting | | microbiome/python- | fre dhutch/docker- |
| (python-pandas) | | pandas:v1.0.3 | python- pandas |

### VIII. Genome Alignments

The process of aligning genes from the geneshot output against a set of reference genomes was executed the Annotation of Microbial Genomes by Microbiome Association (AMGMA) tool. This tool aligns genes from the geneshot gene catalog against a user-specified set of microbial genomes and then calculates summary metrics for each genome including the proportion of the genome which is aligned by genes which are associated with a given parameter. The genome alignment figures show the position of the genome alignment for the set of genes contained in the CAG of interest, as well as the location and annotation of genes recorded in GFF format within the NCBI RefSeq database. Alignment is performed using DIAMOND (using the dependencies shown in Table 3) with the complete set of Python code used to aggregate alignment results available at https://github.com/FredHutch/AMGMA/. An example of alignment to de novo assembled contigs, performed with the same set of steps, is shown in FIG. 17.

### IX. Datasets Used for Analysis

The data analyzed consisted of the publicly available FASTQ records and associated metadata available for the BioProjects PRJEB2289312 (n=25), PRJNA39790614 (n=44), and PRJNA39974213 (n=172). The public metadata for PRJEB2289312 did not include any annotation of ICI response phenotype, and so was omitted from the statistical model used to analyze CAG abundances. However, the CAG abundance data for that set of samples is present in the raw results from the geneshot results.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A system, comprising:
one or more processors; and
one or more computer readable hardware storage devices that comprise computer executable instructions executable by at least one of the one or more processors to cause the computer system to:
identify a set of physical features from a dataset comprising:
physical measurements of a first plurality of bacterial strains in a plurality of microbiome samples from a plurality of subjects, respectively;
phenotypic data comprising a phenotypic feature for each subject of the plurality of subjects; and
physical measurements of a second plurality of bacterial strains;
determine a frequency of each physical feature of the set of physical features;
determine an association value for each physical feature of the set of physical features with the phenotypic feature;
identify a subset of bacterial strains of the second plurality of bacterial strains that is associated with the phenotypic feature, the subset of bacterial strains comprising a first bacterial strain of the second plurality of bacterial strains; and
output the subset of bacterial strains.

2. The system of claim 1, wherein the determining the frequency of each physical feature comprises aligning portions of the sequencing information and filtering to remove multi-mapping reads.

3. A system, comprising:
one or more processors; and
one or more computer readable hardware storage devices that comprise computer executable instructions executable by at least one of the one or more processors to cause the computer system to:
identify a set of physical features from physical measurements of a first bacterial strain;
determine an association value for each physical feature of the set of physical features with a plurality of phenotypic features by determining a frequency of each physical feature of the set of physical features in a dataset comprising;
physical measurements of a plurality of microbiome samples from a plurality of subjects, respectively;
phenotypic data comprising the phenotypic feature for each subject of the plurality of subjects; and
physical measurements of a plurality of bacterial strains; and
identify a phenotypic feature of the plurality of phenotypic features that is associated with the first bacterial strain based at least on the association value; and
output the phenotypic feature.

4. The system of claim 3, wherein the determining the frequency of each physical feature comprises aligning portions of the sequencing information and filtering to remove multi-mapping reads.

5. A system, comprising:
one or more processors; and
one or more computer readable hardware storage devices that comprise computer executable instructions executable by at least one of the one or more processors to cause the computer system to:
receive first physical measurements of a first plurality of bacterial strains in a microbiome sample from a subject;
identify a first set of physical features from the first physical measurements;
determine an association value for each physical feature of the first set of physical features with a plurality of phenotypic features, the association value being based on a frequency of a second set of physical features in a dataset comprising:
physical measurements of a second plurality of bacterial strains in a plurality of microbiome samples from a plurality of subjects, respectively;
phenotypic data comprising a phenotypic feature for each subject of the plurality of subjects; and
physical measurements of a third plurality of bacterial strains;
identify a phenotypic feature of the plurality of phenotypic features that is associated with the first plurality of bacterial strains based at least on the association value; and
output the phenotypic feature.

6. The system of any one of claims 1-5, wherein the determining an association value comprises forming co-abundant gene groups (CAGs) by grouping genes by average linkage clustering.

7. The system of any one of claims 1-6, wherein each physical feature of the set of physical features comprises a gene.

8. The system of any one of claims 1-6, wherein each physical feature of the set of physical features comprises a genomic island.

9. The system of claim 8, wherein the genomic island ranges from about 10 to about 35 kilobases in size.

10. The system of any one of claims 1-9, wherein the physical measurements comprise sequencing information.

11. The system of any one of claims 1-10, wherein the phenotypic feature is a BMI ranging from 18.5 to 24.9.

12. The system of any one of claims 1-10, wherein the phenotypic feature is a BMI of at least 25.0.

13. The system of any one of claims 1-10, wherein the phenotypic feature is a cancer.

14. The system of claim 13, wherein the cancer is responsive to a treatment regimen.

15. The system of claim 14, wherein the treatment regimen comprises immune checkpoint inhibitor (ICI)-based therapy.

16. The system of any one of claims 13-15, wherein the cancer is a hematologic cancer.

17. The system of any one of claims 13-15, wherein the cancer is a solid tumor.

18. The system of any one of claims 13-15, wherein the cancer is melanoma.

19. The system of any one of claims 1-10, wherein the phenotypic feature is colorectal cancer, Crohn's disease, irritable bowel syndrome, non-alcoholic fatty liver disease (NAFLD), nonalcoholic steatohepatitis (NASH), pancreatic cancer, liver cancer, or stomach cancer.

20. The system of any one of claims 1-10, wherein the phenotypic feature is colorectal cancer.

21. The system of any one of claims 1-10, wherein the phenotypic feature is Crohn's disease.

22. A method, comprising:
identifying a set of physical features from a dataset comprising:
physical measurements of a first plurality of bacterial strains in a plurality of microbiome samples from a plurality of subjects, respectively;
phenotypic data comprising a phenotypic feature for each subject of the plurality of subjects; and
physical measurements of a second plurality of bacterial strains;
quantifying a frequency of each physical feature of the set of physical features;
determining an association value for each physical feature of the set of physical features with the phenotypic feature;
identifying a subset of bacterial strains of the second plurality of bacterial strains that is associated with the phenotypic feature, the subset of bacterial strains comprising a first bacterial strain of the second plurality of bacterial strains; and
outputting the subset of bacterial strains.

23. A method, comprising:
identifying a first set of physical features from physical measurements of a first bacterial strain;
determining an association value for each physical feature of the first set of physical features with a plurality of phenotypic features by determining a frequency of each physical feature of the set of physical features in a dataset comprising;
physical measurements of a plurality of microbiome samples from a plurality of subjects, respectively;
phenotypic data comprising the phenotypic feature for each subject of the plurality of subjects; and
physical measurements of a plurality of bacterial strains; and
identifying a phenotypic feature of the plurality of phenotypic features that is associated with the first bacterial strain based at least on the association value; and
outputting the phenotypic feature.

24. A method, comprising:
identifying a set of physical features from a dataset comprising:
physical measurements of a first plurality of bacterial strains in a plurality of microbiome samples from a plurality of subjects, respectively;
phenotypic data comprising a phenotypic feature for each subject of the plurality of subjects; and
physical measurements of a second plurality of bacterial strains;
determining a frequency of each physical feature of the set of physical features;
comparing the physical features to a reference database;
determining an association value for each physical feature of the set of physical features with the phenotypic feature; and
identifying genomic islands that contain the physical features.

25. The method of any one of claims 22-24, wherein the determining the frequency of each physical feature comprises aligning portions of the sequencing information and filtering to remove multi-mapping reads.

26. A method, comprising:
receiving first physical measurements of a first plurality of bacterial strains in a microbiome sample from a subject;
identifying a first set of physical features from the first physical measurements;
determining an association value for each physical feature of the first set of physical features with a plurality of phenotypic features, the association value being based on a frequency of a second set of physical features in a dataset comprising:
physical measurements of a second plurality of bacterial strains in a plurality of microbiome samples from a plurality of subjects, respectively;
phenotypic data comprising a phenotypic feature for each subject of the plurality of subjects; and
physical measurements of a third plurality of bacterial strains;
identifying a phenotypic feature of the plurality of phenotypic features that is associated with the first plurality of bacterial strains based at least on the association value; and
outputting the phenotypic feature.

27. The method of any one of claims 22-26, further comprising determining the frequency of each physical feature comprising aligning portions of the sequencing information and filtering to remove multi-mapping reads.

28. The method of any one of claims 22-27, wherein the determining an association value comprises forming co-abundant gene groups (CAGs) by grouping genes by average linkage clustering.

29. The method of any one of claims 22-28, wherein each physical feature of the set of physical features comprises a gene.

30. The method of any one of claims 22-28, wherein each physical feature of the set of physical features comprises a genomic island.

31. The method of claim 30, wherein the genomic island ranges from about 10 to about 35 kilobases in size.

32. The method of any one of claims 22-31, wherein the physical measurements comprise sequencing information.

33. The method of any one of claims 22-32, wherein the phenotypic feature is a BMI ranging from 18.5 to 24.9.

34. The method of any one of claims 22-32, wherein the phenotypic feature is a BMI of at least 25.0.

35. The method of any one of claims 22-32, wherein the phenotypic feature is a cancer.

36. The method of claim 35, wherein the cancer is responsive to a treatment regimen.

37. The method of claim 36, wherein the treatment regimen comprises immune checkpoint inhibitor (ICI)-based therapy.

38. The method of any one of claims 35-37, wherein the cancer is a hematologic cancer.

39. The method of any one of claims 35-37, wherein the cancer is a solid tumor.

40. The method of any one of claims 35-37, wherein the cancer is melanoma.

41. The method of any one of claims 22-32, wherein the phenotypic feature is colorectal cancer, Crohn's disease, irritable bowel syndrome, non-alcoholic fatty liver disease (NAFLD), nonalcoholic steatohepatitis (NASH), pancreatic cancer, liver cancer, or stomach cancer.

42. The method of any one of claims 22-32, wherein the phenotypic feature is colorectal cancer.

43. The method of any one of claims 22-32, wherein the phenotypic feature is Crohn's disease.

44. A method of treating a disease in a subject with a treatment regimen based on the presence of the subset of bacterial strains in a sample from the subject, the subset of bacterial strains being identified by the method of any one of claim 22, 24, or 26-42.

45. A method of treatment of a disease in a subject in need thereof, the method comprising:
administering a treatment regimen to the subject, wherein the subset of bacterial strains is found in a sample from the subject.

46. The method of claim 44, wherein the subset of bacterial strains is identified by the method of any one of claim 22, 24, 25, or 27-43.

47. The method of any one of claims 44-46, wherein the disease is a cancer.

48. The method of claim 47, wherein the cancer is a hematologic cancer.

49. The method of claim 47, wherein the cancer is a solid tumor.

50. The method of claim 47, wherein the cancer is melanoma.

51. The method of any one of claims 44-46, wherein the disease is colorectal cancer, Crohn's disease, irritable bowel syndrome, non-alcoholic fatty liver disease (NAFLD), nonalcoholic steatohepatitis (NASH), pancreatic cancer, liver cancer, or stomach cancer.

52. The method of any one of claims 44-46, wherein the disease is colorectal cancer.

53. The method of any one of claims 44-46, wherein the disease is Crohn's disease.

54. The method of any one of claims 44-52, wherein the treatment regimen comprises immune checkpoint inhibitor (ICI)-based therapy.
